(19) United States
(12) Patent Application Publication
(10) Pub. No.: US 2025/0340649 A1
(43) Pub. Date: Nov. 6, 2025

(54) SINGLE-DOMAIN HIGH AFFINITY ANTIBODIES AND METHODS OF USE THEREOF

(71) Applicant: Rutgers, The State University of New Jersey, New Brunswick, NJ (US)

(72) Inventors: Arash HATEFI, Metuchen, NJ (US); Shahryar Khoshtinat NIKKHOI, Piscataway, NJ (US)

(73) Assignee: Rutgers, The State University of New Jersey, New Brunswick, NJ (US)

(21) Appl. No.: 18/724,449

(22) PCT Filed: Dec. 16, 2022
(86) PCT No.: PCT/US2022/081806

§ 371 Date: Jun. 26, 2024

(51) Int. Cl. C07K 16/28; A61K 49/16; A61P 35/00; C07K 16/32; G01N 1/30; G01N 21/64; G01N 33/543; G01N 33/574

(52) U.S. Cl. CPC C07K 16/283; A61K 49/16; A61P 35/00; C07K 16/32; G01N 1/30; G01N 21/6458; G01N 21/6486; G01N 33/543; G01N 33/57492; A61K 2123/00; C07K 2317/31; C07K 2317/33; C07K 2317/569; C07K 2317/732; C07K 2319/21; C07K 2319/41; G01N 2333/82

(57) ABSTRACT

The present application relates to single-domain antibodies and constructs comprising the single-domain antibodies, both of which exhibit affinity and specificity toward the CD16a activating receptor on natural killer cells and antigens on cancer cells, bacteria, parasites, or viruses.

### STATEMENT OF GOVERNMENT SUPPORT

This invention was made with government support under grant number HL150852 awarded by the National Institutes of Health. The government has certain rights in the invention.

### TECHNICAL FIELD

The present application relates to single-domain antibodies and constructs comprising the single-domain antibodies, both of which exhibit affinity and specificity toward the CD16a activating receptor on natural killer cells or antigens on cancer cells, bacteria, parasites, or viruses.

### SEQUENCE LISTING

The instant application contains a Sequence Listing which has been filed electronically in XML format and is hereby incorporated by reference in its entirety. Said XML copy, created on Feb. 24, 2023 is named 10491_010848-US2_SL and is 77.212 bytes in size.

### BACKGROUND

The crystallizable fragment (Fc) γ receptors (FcγRs) which are expressed on the surface of leucocytes bind to immunoglobulin G (IgG) antibodies and are essential for the efficacy of many antibody-based drugs. The FcγRs are divided into activating receptors (FcγRI/CD64, FcγRIIa/CD32a, FcγRIIc/CD32c, FcγRIIIa/CD16a, and FcγRIIIb/CD16b) and inhibitory receptors (FcγRIIb/CD32b) (1). These receptors bind to IgGs, although, with different affinities (2). CD16a is low-affinity and the primary receptor for the Fc region of monoclonal antibodies (mAbs) and the only FcγR expressed on the surface of natural killer (NK) cells. CD16a on NK cells binds the antibody-coated cells (e.g., cancer cells) triggering an antibody-dependent cell cytotoxicity (ADCC).

Owing to this function, CD16a-expressing NK cells are currently being investigated in clinical trials for cancer therapy (e.g., NCT04673617 and NCT03383978). It is well established that by increasing the binding affinity of CD16a toward the antibody Fc region, the NK cell cytotoxicity and clinical outcomes can be significantly improved (1, 3). However, current antibodies in this area generally lack specificity to CD16a, which can hamper therapeutic efficacy and result in off-target toxicities. For example, antibodies that also bind to CD16b activating receptor (expressed on neutrophils) have been shown to restrict the ADCC activity of NK cells against cancer cells (4). Furthermore, non-specific binding of antibodies to inhibitory CD32b receptor (expressed on B cells) has also been shown to inhibit B cell maturation and macrophage activation (4, 5). CD32b is also expressed on a subset of CD8⁺ T cells, which could restrict T cell survival by activating Caspase 3 and 7 pathways (6).

CD16a-expressing NK cells can also be involved with immune responses to pathogen, such as bacteria, parasites, or viruses, and thus improvements in ADCC activity of NK cells can also improve clinical outcomes for patients with bacterial or viral infections.

The present application addresses the aforementioned challenges and other problems related to activating and redirecting natural killer cells to effectively target surface antigens and lyse target cells including cancer cells, bacteria, parasites, or viruses.

### SUMMARY

In a first aspect, a construct is provided. The construct comprises a first single-domain antibody, having an amino acid sequence of one of SEQ ID NOs: 14 and 15, that exhibits specificity and affinity towards the CD16a receptor on the surface of natural killer (NK) cells without cross reactivity with CD16b (e.g., CD16b-NA1) or CD32b. The construct also comprises a second single-domain antibody having an amino acid sequence that exhibits specificity and affinity toward an antigen associated with a cancer cell, bacteria, parasite, or virus, wherein the first and second single-domain antibodies are fused with each other with or without a linker.

In another aspect, the antigen is associated with a cancer cell, and wherein the antigen is selected from the group consisting of: HER2, HER1, HER3, HER4, EGFR, VEGFR, CD47, FGFR, carcinoembryonic antigen (CEA), Bladder Tumor Antigen (BTA), CA125, PDGFR, IGFR, CA15-3/CA27.29, CA19-9, CA27.29, programmed death ligand 1 (PD-L1), PD-L2, CTL4, CD3, CD19, CD20, CD22, CD25, CD27, CD30, CD33, CD37, CD38, CD40, CD48, CD52, B7-H3, MICA family, RAET1/ULBP family, HLA-E, TIM-3, LAG-3, V-domain Ig suppressor of T cell activation (VISTA), HVEM, ICOS, 4-1BB, OX40, RANKL and GITR, epithelial and mesenchymal markers of circulating tumor cells, Prostatic Acid Phosphatase (PAP), prostate-specific antigen (PSA), soluble mesothelin-related peptides (SMRP), somatostatin receptor (SR), Urokinase plasminogen activator (uPA), plasminogen activator inhibitor (PAI-1), TCR (e.g., MHC class I or class II molecules), A2a Receptor, glioma-associated antigen, carcinoembryonic antigen (CEA), beta-human chorionic gonadotropin, RAGE-1, MN-CAIX, RU1, RU2 (AS), intestinal carboxyl esterase, mut hsp70-2, M-CSF, prostase, prostate-specific antigen (PSA), PAP, NY-ESO-1, LAGE-1a, prostein, PSMA, prostate-carcinoma tumor antigen-1 (PCTA-1), MART-1, MAGE, tyrosinase, TRP-1, TRP-2 BAGE, GAGE-1, GAGE-2, RAGE, pl5, ELF2M, neutrophil elastase, ephrinB2, IGF-I receptor, E2A-PRL, H4-RET, IGH-IGK, MYL-RAR, TSP-180, p185erbB2, p180erbB-3, nm-23HI, TAG-72, CA 19-9, CA 72-4, CAM 17.1, NuMa, beta-Catenin, CDK4, Mum-1, p 15, p 16, 43-9F, 5T4, 791Tgp72, beta-HCG, BCA225, BTAA, CA 15-3CA 27.29BCAA, CA 195, CA 242, CA-50, CAM43, CD68P1, CO-029, G250, Ga733EpCAM, HTgp-175, M344, MA-50, MG7-Ag, MOV18, NB/70K, NY-CO-1, RCAS 1, SDCCAG16, TA-90Mac-2 binding protein\cyclophilin C-associated protein, TAAL6, TAG72, TLP, and TPS.

In another aspect, the antigen is associated with bacteria, and wherein the antigen is selected from the group consisting of polysaccharides or peptide antigens associated with P. aeruginosa, S. aureus, Clostridium difficile, Acinetobacter baumannii, and Klebsiella pneumonia.

In another aspect, the antigen is associated with a virus, and wherein the antigen is selected from the group consisting of Epstein Barr virus antigens EBVA, human papillomavirus (HPV) antigens E6 and E7, coronavirus surface antigens, influenza virus surface antigens, and HIV surface antigens.

In another aspect, the antigen is associated with a parasite, and wherein the antigen is selected from the group consisting of antigens associated with malaria, Leishmaniasis, Chagas Disease, Toxoplasmosis, Schistosomiasis, Cysticercosis, and Strongyloidiasis.

In another aspect, the first single-domain antibody comprises the amino acid sequence of SEQ ID NO: 14 and the second single-domain antibody comprises the amino acid sequence of SEQ ID NO: 6.

In another aspect, the second single-domain antibody comprises an amino acid sequence of at least one of SEQ ID NOs: 3-11, and wherein the second single-domain antibody exhibits selectivity and affinity towards HER2 and facilitates recognition of HER2-expressing cancer cells.

In a further aspect, the HER2-expressing cancer cells are ovarian cancer cells, breast cancer cells, gastric cancer, gastroesophageal cancer, cervical cancer cells, bladder cancer cells, gallbladder cancer cells, testicular cancer cells, uterine cancer cells, or any other HER2-expressing cancer cells.

In another aspect, the amino acid sequence of the first single-domain antibody exhibits high affinity towards the CD16a receptor of the NK cells.

In another aspect, the amino acid sequence of the second single-domain antibody exhibits high affinity towards the antigen associated with the cancer cell, bacteria, parasite, or virus.

In another aspect, the first and second single-domain antibodies are fused with a linker.

In a further aspect, the linker is a human muscle aldolase (HMA) linker.

In another aspect, the construct further comprises at least one additional single-domain antibody having an amino acid sequence that exhibits affinity and specificity toward another epitope on the same antigen or on another antigen and fused to at least one of the first or second single-domain antibody with or without a linker.

In a further aspect, the at least one additional single-domain antibody is the same type of antibody as the first single-domain antibody.

In a further aspect, the at least one additional single-domain antibody is the same type of antibody as the second single-domain antibody.

In a second aspect, a single-domain antibody is provided. The single-domain antibody comprises an amino acid sequence of at least one of SEQ ID NOs: 14 and 15, wherein the single-domain antibody selectively and with high affinity binds to a CD16a activating receptor on the surface of natural killer (NK) cells, without cross reactivity with CD16b-NA1 or CD32b.

In a third aspect, a single-domain antibody is provided, where the single-domain antibody comprises an amino acid sequence of at least one of SEQ ID NOs: 3-11, wherein the single-domain antibody exhibits selectivity and high affinity towards HER2 and facilitates recognition of HER2-expressing cancer cells.

In a fourth aspect, a method for inhibiting HER2-positive cancers in a subject is provided. In the method, an effective amount of a construct as mentioned above is administered to the subject, wherein the construct activates NK cells in the subject to recognize target HER2-positive cancer cells in the subject.

In a fifth aspect, a method of performing an ELISA assay using a single-domain antibody or a construct as mentioned above is provided. In the method, a sample comprising one or more antigens is immobilized on a solid support, wherein the one or more antigens are selected from HER2 and CD16a. The single-domain antibody is applied over a surface of the sample, wherein the single-domain antibody acts as a primary antibody. A secondary antibody is applied over the surface of the sample, wherein the secondary antibody is linked to an enzyme and is configured recognize the single-domain antibody. A substance containing a substrate of the enzyme's substrate is added to the sample. The sample is then examined to determine whether there is binding between the single-domain antibody and the one or more antigens, wherein if there was binding by the single-domain antibody to the one or more antigens, the subsequent reaction produces a detectable signal in the sample.

In a sixth aspect, a method of performing a flow cytometry assay using a single-domain antibody as mentioned above is provided. In the method, a sample containing cancer cells and the single-domain antibody is suspended in a fluid. A secondary antibody linked to a fluorescent probe that can bind to the single-domain antibody is applied to the sample. The fluid comprising the sample is injected into a flow cytometer instrument. The sample is then analyzed with a flow cytometry analyzer, and then it is determined whether the cancer cells are HER2+ cancer cells.

In a seventh aspect, a cell imaging method using a single-domain antibody as mentioned above is provided. In the method, a sample comprising suspected cancer cells is fixed on a slide, and the single-domain antibody is applied to the sample. A secondary antibody linked to a fluorescent probe that can bind to the single-domain antibody is applied to the sample. The sample is then examined via a confocal or fluorescent microscope to detect a presence or absence of HER2 expression on the surface of the suspected cancer cells. In another aspect, the fluorescently-labeled secondary antibody is an anti-histag antibody or an anti-C-myc tag antibody.

In an eighth aspect, an in vivo cell tracking and imaging method for tracking allogenic or autologous NK cells in a subject using a single-domain antibody as mentioned above is provided. In the method, an imaging substance conjugated to the single-domain antibody is administered to the subject. A whole body-imaging method of the subject is performed to produce an image, and the anatomical location of the NK cells in the image is identified. In another aspect, the whole body-imaging method is selected from the group consisting of: magnetic resonance imaging (MRI), positron emission tomography (PET), computed tomography (CT), and single photon emission computed tomography (SPECT).

In a ninth aspect, an in vivo cancer phenotyping method for identifying HER2-expressing cancer lesions in a subject is provided. In the method, an imaging substance conjugated to a single-domain antibody as mentioned above is administered to the subject. A tumor-imaging method of the subject is performed to produce an image, and HER2-expressing cancer lesions are identified in the image. In another aspect, the tumor-imaging method is selected from the group consisting of: magnetic resonance imaging (MRI), positron emission tomography (PET), computed tomography (CT), and single photon emission computed tomography (SPECT).

### BRIEF DESCRIPTION OF THE DRAWINGS

FIG. 1A. Schematics of structural differences between conventional IgG and camelid IgG. While the Fab fragment of conventional IgG consists of both heavy chain and light chain, camelid IgG is consisted of only heavy chain and lacks light chain.

FIGS. 1B-1D. FIG. 1B) Schematic representation of an anti-CD16a VHH and an anti-antigen VHH bound to one another via a linker. FIG. 1C) Schematic representation of a BIKE comprising the anti-CD16a VHH and the anti-antigen VHH, where the BiKE activates an NK cell to recognize a target (cancer cell, bacteria, virus). FIG. 1D) Schematic representation of various configurations of one or more anti-CD16a VHHs bound to one or more anti-antigen VHHs via a linker. Two or more anti-CD16a VHHs can be engineered in tandem to recognize two or more different epitopes on CD16a antigen. Similarly, two or more anti-antigen VHHs can be engineered in tandem to recognize two or more different epitopes on target antigen.

FIG. 2. A timeline and method used to immunize llama for the generation of VHHs against HER2 protein.

FIGS. 3A-3B. FIG. 3A) SDS-PAGE analysis of the purified HER2 antigen with theoretical molecular weight of 72,633 Da. FIG. 3B). The level of IgG in serum of llama before and after immunization with HER2 protein as measured by ELISA.

FIGS. 4A-4B. FIG. 4A) PBMCs were isolated using Ficoll-Paque method. Then, the RNAs were extracted, cDNA library was generated, genes amplified, and then cloned into pMECS-GG phagemids. The phagemids were used in phage display and the screened candidates were used to infect TG1 bacteria. Colonies were selected, grown, lysed, and the lysates removed. FIG. 4B) The lysates were then used in ELISA to screen for VHHs with the highest affinity to HER2 and with negligible binding to HER1, HER3, and HER4. Bovine serum albumin (BSA) and skim milk were used as controls.

FIG. 5A. The SDS-PAGE analysis of the purified c-myc/histagged anti-HER2 VHHs.

FIGS. 5B-5E. The flow cytometry histograms of HER2+ (BT474 and SKOV-3, FIGS. 5B and 5C, respectively) and HER2⁻ (MDA-MB-231 and OVASC-1, FIGS. 5D and 5E, respectively) cancer cells labeled with anti-HER2 VHHs, Trastuzumab, and Pertuzumab. The equimolar binding sites of purified VHHs and FDA-approved anti-HER2 monoclonal antibodies (Trastuzumab and Pertuzumab) were used to measure the HER2 expression on the surface of HER2+ cancer cells. HER2⁻ cancer cells were used as negative controls. The flow cytometry data showed that the selected anti-HER2 VHHs can recognize HER2 on the surface of HER2+ cancer cells without binding to HER2⁻ cancer cells.

FIGS. 6A-6B. The evaluation of the toxicity of the selected anti-HER2 VHHs, Trastuzumab, and Pertuzumab to HER2+BT474 (FIG. 6A) and SKOV-3 (FIG. 6B) cancer cells. The cell toxicity was measured by WST-1 cell toxicity assay and data are shown as mean±s.d.

FIGS. 7A-7B. Determination of the anti-HER2 VHH affinity and binding kinetics by using a biolayer interferometer. The KD (affinity), Kₒₙ (association constant), and K_{off} (dissociation constant) were determined by using the Octet Data Analysis HT 11.1 software. E5 and A1 clones were selected as high performing VHH candidates.

FIGS. 8A-8B. Confocal microscopy images of the SKOV-3 cells treated with anti-HER2 VHHs (E5 and A1 clones) and imaged at different time points. The cell nucleus is labeled with DAPI (blue) and VHH with FITC (green). The overlay images from the top view (mid slice from the Z-stacks) show time-dependent binding to cells, whereas the side view images (edge) show the internalization of the fluorescent-labeled VHHs. The results of this experiment showed that both A1 (FIG. 8A) and E5 (FIG. 8B) clones started to internalize as early as 1 hour and the internalization process completed in 3 to 5 hours. Published data show that Trastuzumab binds and internalizes into HER2+ cancer cells as early as 4 hours.

FIGS. 9A-9B. FIG. 9A) The SDS-PAGE analysis of purified rCD16a (20 μg). FIG. 9B) The level of IgG in serum of llama before and after immunization with rCD16a protein as measured by ELISA.

FIGS. 10A-10C. FIG. 10A: PBMCs were isolated using Ficoll-Plaque method. Then, the RNAs were extracted, cDNA library was generated, genes amplified, and then cloned into pMECS-GG phagemids. The phagemids were used in phage display and the screened candidates were used to infect TG1 bacteria. Colonies were selected, grown, lysed, and the lysates removed. FIG. 10B: Evaluation of the specificity of the anti-CD16a VHHs from the periplasmic extracts toward CD16a and CD16b antigens by using ELISA. Skim milk was used as control. FIG. 10C: Evaluation of the binding affinity of the anti-CD16a VHHs in periplasmic extract toward CD16a antigen after 1280 fold dilution.

FIG. 11. Evaluation of the specificity of the C1 and E3 anti-CD16a VHHs toward CD16a antigen. CD16b-NA1, CD16-NA2, CD32b, and skim milk were used as antigen controls, whereas commercially available 3G8 (anti-CD16a/b mAb) and eBioCB16 (anti-CD16a/b mAb) mAbs were used as antibody controls. This figure shows that the C1 and E3 anti-CD16a VHHs bind specifically to CD16a without cross-reactivity with CD1b-NA1 and CD32b.

FIGS. 12A-12E. Evaluation of the specificity of the C1 and E3 anti-CD16a VHHs toward CD16a antigen by flow cytometry using NK92 (CD16+) cells (FIGS. 12A-12B), neutrophils (FIGS. 12C-12D), and B cells (FIG. 12E). The percent positive (PE+), percent negative (PE⁻), and the mean fluorescent intensity (MFI) of labeled cells are shown. This figure shows the specificity of C1 and E3 anti-CD16a VHHs towards CD16a receptor.

FIGS. 13A-13E. Determination of the anti-CD16a VHH affinity, binding kinetics, and specificity by using a biolayer interferometer. The KD (affinity), Kₒₙ (association constant), and K_{off} (dissociation constant) were determined by using the Octet Data Analysis HT 11.1 software. FIGS. 13A-13B) Binding affinity of anti-CD16a C1 and E3 clones toward CD16a antigen. FIGS. 13C-13D) Binding affinity of anti-CD16a C1 and E3 clones toward CD16b-NAI antigen. FIG. 13E) Epitope mapping for C1 VHH and mAbs (trastuzumab and pertuzumab) against CD16a antigen. This figure shows the high affinities of anti-CD16a VHHs toward CD16a antigen with significantly less affinities toward CD16b-NA1. It also shows C1 VHH binds to a different epitope on CD16a than trastuzumab and pertuzumab.

FIGS. 14A-14C. FIG. 14A) The schematic representation of anti-HER2 VHH (E5 clone) fused with anti-CD16a VHH (C1 clone) via a human muscle aldolase (HMA) linker to generate BiKE: HER2/CD16a. FIG. 14B) The SDS-PAGE analysis of expressed and purified BiKE: HER2/CD16a with theoretical molecular weight of 33.48 kDa. FIG. 14C) Liquid chromatography-Mass Spectroscopy (LC-MS) graph of the purified BiKE: HER2/CD16a (˜1 mg/ml) showing the peptide as monomer without the presence of dimer or multimer.

FIGS. 15A-15D. FIG. 15A) Comparison of the binding of anti-CD16a VHH with BiKE: HER2/CD16a by ELISA. FIG. 15B) Comparison of the binding of anti-HER2 VHH with BiKE: HER2/CD16a by ELISA. FIG. 15C) Comparison of the binding of anti-CD16a VHH with BIKE: HER2/CD16a by flow cytometry in NK92 (CD16+) cells. FIG. 15D) Comparison of the binding of anti-HER2 VHH with BIKE: HER2/CD16a by flow cytometry in SKOV-3 (HER2+) cells. These figures show that the fusion of anti-CD16a VHH with anti-HER2 VHH did not affect its binding affinity toward the target antigens.

FIG. 16A-16C. Binding affinity of E5C1 BiKE (BIKE: CD16a/HER2) toward CD16a, CD16b-NA1, and CD16b-NA2antigens.

FIGS. 17A-17D. FIG. 17A) Schematic representation of antibody-directed cell cytotoxicity. BiKE, Trastuzumab, and Pertuzumab activate NK cells to recognize target cancer cells. FIGS. 17B-17D) SKOV-3, BT474, and JIMT-1 cells treated with haNK92 cells at different E:T (NK:Target cells) ratios under non-adherent cell conditions. ADCC was measured after treating cancer cells with haNK92 cells in combination with Pertuzumab (Prz.), Trastuzumab (Trz.), or BiKE. This figure shows that BiKE induced significantly higher ADCC in comparison to Trz. or Prz.

FIGS. 18A-18K. FIGS. 18A-C) Measurement of ADCC under adherent conditions in three HER2+ cancer cell lines using laNK92 cells in combination with BiKE or Trastuzumab at different E:T ratios but fixed antibody concentration (100 nM). FIGS. 18D-F) Measurement of ADCC in three HER2+ cancer cell lines using laNK92 cells in combination with BiKE or Trastuzumab at different antibody concentrations but fixed E:T ratio of 4. FIGS. 18G-J) Measurement of IFN-γ, TNF-α, Perforin, and Granzyme B after incubation of SKOV-3 cells with laNK92 cells in the presence of BiKE or Trastuzumab (Trz.) using ELISA. FIG. 18K) Measurement of change in CD107a expression (laNK92 degranulation) at different antibody concentrations using flow cytometry. The data are shown as mean±s.d. (*t-test, p<0.05, n.s.: not significant).

FIGS. 19A-19K. FIGS. 19A-C) Measurement of ADCC under adherent conditions in HER2+ cancer cell lines using haNK92 cells in combination with BiKE or Trastuzumab at different E:T ratios but fixed antibody concentration (100 nM). FIGS. 19D-F) Measurement of ADCC in HER2+ cancer cell lines using haNK92 cells in combination with BiKE or trastuzumab at different antibody concentrations but fixed E:T ratio of 4. FIGS. 19G-J) Measurement of IFN-γ, TNF-α, Perforin, and Granzyme B after incubation of SKOV-3 cells with haNK92 cells in the presence of BiKE or trastuzumab (Trz.) using ELISA. FIG. 19K) Measurement of change in CD107a expression (haNK92 degranulation) at different antibody concentrations using flow cytometry. The data are shown as mean±s.d. (*t-test, p<0.05).

FIG. 20: Measurement of ADCC under adherent conditions in HER2+ SKOV-3 cells incubated with laNK92 cells for four hours in the presence of BiKE (10 pM), trastuzumab (Trz.) (10 pM), pertuzumab (Prz.) (10 pM), trastuzumab (10 pM)+pertuzumab (10 pM), BiKE (10 pM)+pertuzumab (10 pM), BiKE (10 pM)+trastuzumab (10 pM), or no antibody (i.e., SKOV3+ laNK92 only) (*t-test, p<0.05).

### DETAILED DESCRIPTION

In accordance with one or more embodiments, the present application relates to single-domain antibodies, constructs comprising two or more single-domain antibodies; and associated cancer cell killing methods, bacteria killing methods, parasite killing methods, virus killing methods, imaging methods and assay methods that utilize the single-domain antibodies and the constructs comprising said single-domain antibodies.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Natural killer” or “NK” cells is a type of cytotoxic lymphocyte that is critical to the innate immune system. NK cells represent approximately 5-20% of all circulating lymphocytes in humans.

“CD16” refers to a type III Fcγ receptor. In humans, it exists in two different forms: FcγRIIIa (“CD16A”) and FcγRIIIb (“CD16B”). “CD16A” is an activating receptor CD16A expressed on the cell surface of NK cells and macrophages. CD16A can trigger the cytotoxic activity of NK cells and macrophages. The affinity of antibodies for CD16A directly correlates with their ability to trigger NK cell activation, thus higher affinity towards CD16A reduces the antibody dose required for activation. CD16B is only expressed on neutrophils.

“CD32”, also known as FcγRII or FCGR2, refers to a surface receptor glycoprotein that can be found on the surface of a variety of immune cells. CD32B, one of three major CD32 subtypes, is an inhibitory surface receptor that is part of a large population of B-cell co-receptors.

The term “HER2” or “human epidermal growth factor receptor 2” refers to an oncogene known to be associated with the development and progression of certain aggressive types of cancers such as breast cancer or ovarian cancer.

The term “histag”, “his-tag” or “polyhistidine-tag” refers to an amino acid motif in proteins that generally consists of six or more histidine (His) residues at the N- or C-terminus of the protein.

The term “imaging agent” or “imaging substance” generally refers to a compound or agent used to increase the contrast of structures or fluids within the body during medical imaging (e.g., PET, MRI). The term “imaging agent” or “imaging substance” can be used interchangeably with the term “contrast agent.”

The terms “VHH”, “nanobody”, and “single-domain antibody” generally refers to an antibody fragment that consists of a single monomeric variable antibody domain, which is able to bind selectively to a specific antigen.

The term “single-domain antibody construct” generally refers to a construct comprising two or more single-domain antibodies, where one or more single-domain antibodies binds to activating receptors (i.e., CD16a) on natural killer cells and one or more single-domain antibodies bind to antigens on cancer cells, bacterial cells, parasites, or viruses. The single-domain antibody construct may be bispecific, trispecific, tetraspecific or multi-specific.

The term “BiKE”, or “Bispecific Killer Cell Engager”, generally refers to a construct comprising two or more single-domain antibodies, where the construct binds to activating receptors (i.e., CD16a) on natural killer cells and macrophages and to antigens on cancer cells, bacterial cells, parasites, or viruses.

The term “peptide”, as used herein, refers to peptides and proteins longer than two amino acids in length that may also incorporate non-amino acid molecules.

The terms “without binding” or “negligible binding” or “without cross reactivity with” are used interchangeably herein and generally refer to binding that is similar to background (control) binding and/or statistically insignificant binding.

The phrases “pharmaceutically acceptable” or “pharmacologically acceptable” refer to molecular entities and compositions that do not produce an adverse, toxic, allergic, inflammatory, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like that are pharmaceutically acceptable as the term is used herein and preferably inert. The use of such media and agents for pharmaceutical active substances is well known in the art. Supplementary active ingredients can also be incorporated into the compositions. Except insofar as any conventional media or agent is incompatible with the active ingredients, its use in therapeutic compositions is contemplated.

The term “effective amount of an imaging agent” as used herein refers to an amount of an imaging agent sufficient to obtain a signal suitable for medical imaging of a portion of the body. Methods of determining the most effective amount of the imaging agent can vary with the composition used, the purpose of the use, and the target cell being imaged. When the imaging agents described herein are co-administered with another agent, the effective amount may be less than when the agent is used alone. Suitable formulations and methods of administering the imaging agents can be readily determined by those of skill in the art.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the disclosure can be administered. In an exemplary embodiment of the present disclosure, to identify subject patients for treatment according to the methods of the disclosure, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present disclosure.

The terms “treat”, “treating” or “treatment” of a state, disorder or condition includes: (a) preventing or delaying the appearance of clinical symptoms of the state, disorder, or condition developing in a person who may be afflicted with or predisposed to the state, disorder or condition but does not yet experience or display clinical symptoms of the state, disorder or condition; or (b) inhibiting the state, disorder or condition, i.e., arresting, reducing or delaying the development of the disease or a relapse thereof (in case of maintenance treatment) or at least one clinical symptom, sign, or test, thereof; or (c) relieving the disease, i.e., causing regression of the state, disorder or condition or at least one of its clinical or sub-clinical symptoms or signs.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, the terms “therapeutically effective amount”, “therapeutically effective dose” and “effective amount” refer to an amount of the compound and compositions which is sufficient to effect beneficial or desired results, that, when administered alone or in combination with an additional therapeutic agent to a cell, tissue, or subject, is effective to cause a measurable improvement in one or more symptoms related to the particular disease or medical condition. A therapeutically effective dose further refers to that amount of the compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount can also result in an improvement in a subjective measure in cases where subjective measures are used to assess disease severity.

### Single-Domain Antibodies

Single-domain antibodies, also known as VHHs (variable domain of the heavy chain of a heavy chain-only antibody) are antibody fragments consisting of a single monomeric variable antibody domain and lack a fragment crystallizable (Fc) region. In addition to lacking a Fc region, VHHs possess unique characteristics including high-target specificity, affinity in (sub-) nanomolar range, high stability, and ease of production in both mammalian and E. coli cells. These characteristics allow them to outperform conventional antibodies for imaging and radiotherapeutic purposes.

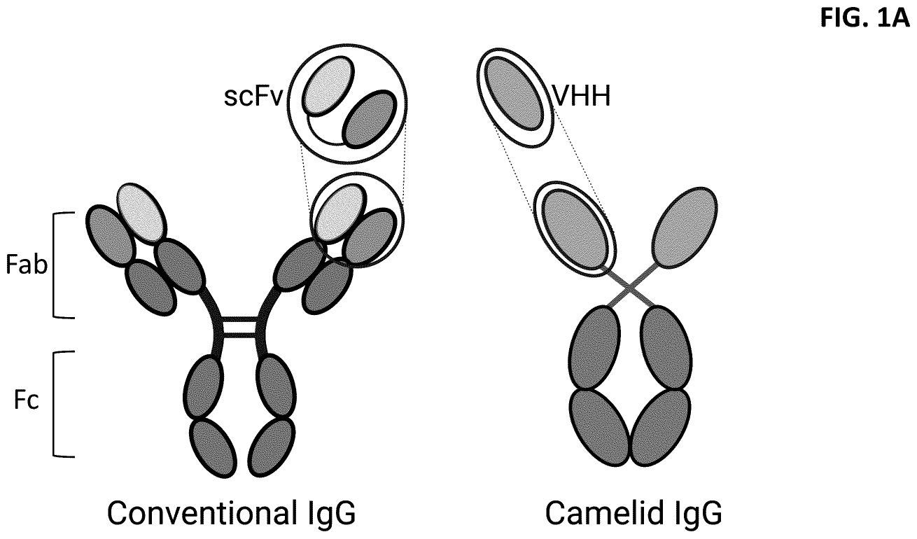

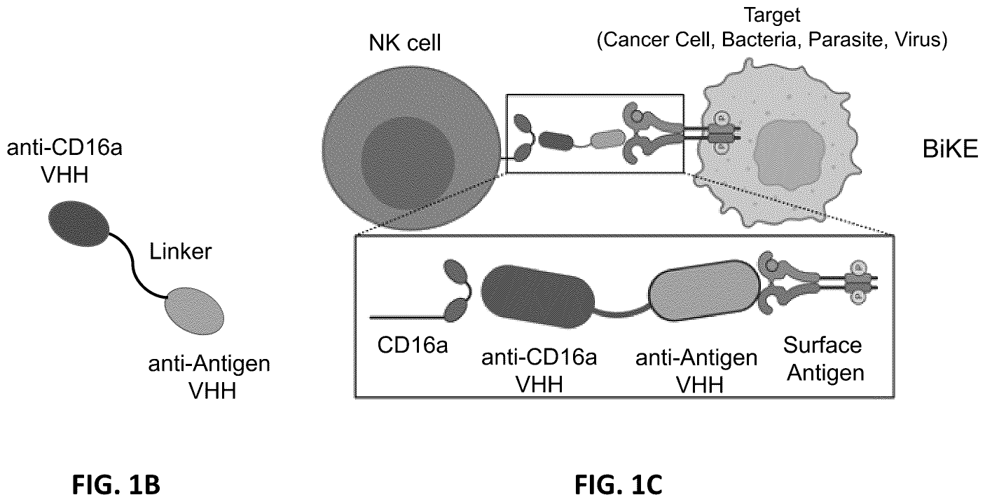

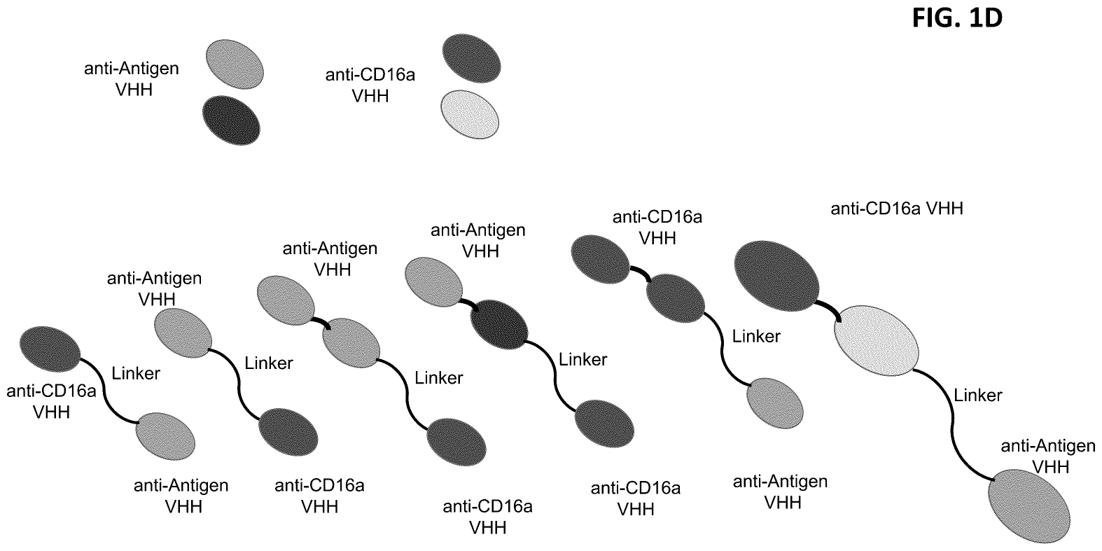

FIG. 1A displays schematics of structural differences between a conventional immunoglobulin (IgG), a type of antibody, and a camelid IgG. While the Fab fragment of conventional IgG consists of both heavy chain and light chain, the camelid IgG consists of only heavy chain and lacks light chain. As shown in FIG. 1A, this Fab fragment with only the heavy chain in the camelid IgG represents a single-domain antibody (VHH).

In accordance with one or more embodiments, the single-domain antibodies (VHHs) of the present application selectively bind with high affinity (sub-nanomolar range) to a) CD16a activating receptors on the surface of natural killer (NK) cells or b) an antigen associated with a cancer cell, bacteria, parasite, or virus. Most antibodies have K_{D} values in the low micromolar (10⁻⁶) to nanomolar (10⁻⁷ to 10⁻⁹) range. High affinity antibodies are generally considered to be in the low nanomolar range (10⁻⁹) with very high affinity antibodies being in the picomolar (10⁻¹²) range. K_{D} values generally correspond to molar concentration (sensitivity) as follows:

| KD value | Molar concentration (sensitivity) |
|---|---|
| 10⁻⁴ to 10⁻⁶ | Micromolar (μM) |
| 10⁻⁷ to 10⁻⁹ | Nanomolar (nM) |
| 10⁻¹⁰ to 10⁻¹² | Picomolar (pM) |
| 10⁻¹³ to 10⁻¹⁵ | Femtomolar (fM) |

In accordance with one or more embodiments of the present application, an antibody with a binding affinity of 100 nM or less is considered “high affinity,” an antibody with a binding affinity of 100-500 nM affinity is considered “medium affinity” and an antibody with a binding affinity of more than 500 nM is considered “medium-low affinity.”

For example, in one or more embodiments, the binding affinity of the single-domain antibodies of the present application towards CD16a or an antigen associated with a cancer cell, bacteria, parasite, or virus is up to 50 nM. In at least one embodiment, the binding affinity of the single-domain antibodies of the present application towards CD16a or an antigen associated with a cancer cell, bacteria or virus is in the range of 750 pM to 850 pM, 850 pM to 950 pM, 950 pM to 1 nM, 1 nM to 1.2 nM, 1.2 nM to 1.4 nM, 1.4 nM to 1.6 nM, 1.6 nM to 1.8 nM, 1.8 nM to 2.0 nM, 2.0 nM to 2.2 nM, 2.4 nM to 2.6 nM, 2.6 nM to 2.8 nM, or 2.8 nM to 3.0 nM. In one or more embodiments, the binding affinity of the single-domain antibodies of the present application toward CD16a or an antigen associated with a cancer cell, bacteria, parasite, or virus is in the range of 3.0 nM to 5.0 nM, 5.0 nM to 10 nM, 10 nM to 15 nM, 15 nM to 20 nM, 20 nM to 25 nM, 25 nM to 30 nM, 30 nM to 35 nM, 35 nM to 40 nM, 40 nM to 45 nM, or 45 nM to 50 nM. As such, the single-domain antibodies of the present application are considered “high affinity” antibodies.

The ability to selectively bind to the CD16a receptors of NK cells and/or other antigens of target cells enhances the ability of NK cells to kill target cells via a mechanism known as antibody-dependent cell cytotoxicity (ADCC). Through ADCC, NK cells can actively lyse a target (e.g., target cell) whose membrane-surface antigens have been bound by specific antibodies.

The ability to selectively bind to the CD16a receptors of macrophages and/or other antigens of target cells enhances the ability of macrophages to kill target cells via a mechanism known as antibody-dependent cell phagocytosis (ADCP). Through ADCP, macrophages can actively lyse a target (e.g., target cell) whose membrane-surface antigens have been bound by specific antibodies.

Moreover, in one or more embodiments, the single-domain antibodies of the present application selectively bind to the CD16a activating receptor on NK cells, but do not exhibit cross reactivity with CD16b-NA1 or CD32b. Specificity of the single-domain antibodies towards CD16a plays a significant role in boosting its therapeutic efficacy and reducing off-target toxicities. In contrast, other antibodies that bind to CD16b activating receptor (expressed on neutrophils) have been shown to restrict the ADCC activity of NK cells against target cells (e.g., cancer cells, bacteria, parasites, viruses). Furthermore, non-specific binding of antibodies to inhibitory CD32b receptor (expressed on B cells) has also been shown to inhibit B cell maturation and macrophage activation. CD32b is also expressed on a subset of CD8+ T cells which could restrict T cell survival by activating Caspase 3 and 7 apoptotic pathways.

In one or more embodiments, the high-affinity and high-specificity single-domain antibodies of the present application, including single-domain antibodies that selectively bind to CD16a (“anti-CD16a VHHs”), can be used for flow cytometry, ELISA, and imaging of tumor infiltrating NK cells.

In one or more embodiments, the single-domain antibodies of the present application exhibit binding affinity and specificity towards antigens associated with a cancer cell. For example, in at least one exemplary embodiment, the single-domain antibody exhibits affinity towards HER2 (i.e., anti-HER2 VHHs) and facilitates recognition of HER2-expressing cancer cells. In one or more embodiments, the present application also discloses methods for identifying HER2-expressing cancer lesions in a subject via a tumor imaging method using the single-domain antibodies of the present application. HER2-expressing cancers (HER2+ cancers) are aggressive and associated with metastasis. As such, anti-HER2 single-domain antibodies and associated methods of the present application provide a reliable approach for identifying such cancers and distinguishing them from non-HER2-expressing lesions. The VHHs and methods of the present application can be used to identify HER2-expressing tumors, such as ovarian tumors, and their metastatic sites, such as lung metastatic sites. Since lung metastasis is the second most frequent metastatic site in ovarian cancer (e.g., HER2+ ovarian cancer), a reliable quantitative diagnostic approach for biological characterization of such metastatic sites is clinically valuable.

In accordance with one or more embodiments, single-domain antibodies of the present application can exhibit binding affinity and specificity towards one or more other antigens associated with cancer cells. For example, in one or more embodiments, the single-domain antibodies can exhibit affinity and specificity towards cancer-related antigens including but not limited to HER1 (anti-HER1 antibodies), HER3 (anti-HER3 antibodies), HER4 (anti-HER4 antibodies), EGFR (anti-EGFR antibodies), VEGFR (anti-VEGFR antibodies), CD47 (anti-CD47 antibodies), FGFR (anti-FGFR antibodies), carcinoembryonic antigen (CEA) (anti-CEA antibodies), Bladder Tumor Antigen (BTA) (anti-BTA antibodies), CA125 (anti-CA125 antibodies), PDGFR (anti-PDGFR antibodies), IGFR (anti-IGFR antibodies), CA15-3/CA27.29 (anti-CA15-3/CA27.29 antibodies), CA19-9 (anti-CA19-9 antibodies), CA27.29 (anti-CA27.29-antibodies), programmed death ligand 1 (PD-L1) (anti-PD-L1 antibodies), PD-L2 (anti-PD-L2 antibodies) CTL4 (anti-CTL4 antibodies), CD3 (anti-CD3 antibodies), CD19 (anti-CD19 antibodies), CD20 (anti-CD20 antibodies), CD22 (anti-CD22 antibodies), CD25 (anti-CD25 antibodies), CD27 (anti-CD27 antibodies), CD30 (anti-CD30 antibodies), CD33 (anti-CD33 antibodies), CD37 (anti-CD37 antibodies), CD38 (anti-CD38 antibodies), CD40 (anti-CD40 antibodies), CD48 (anti-CD48 antibodies), CD52 (anti-CD52 antibodies), B7-H3 (anti-B7-H3 antibodies), TIM-3 (anti-TIM-3 antibodies), LAG-3 (anti-LAG-3 antibodies), V-domain Ig suppressor of T cell activation (VISTA) (anti-VISTA antibodies), HVEM (anti-HVEM antibodies), ICOS (anti-ICOS antibodies), 4-1BB (anti-4-1BB antibodies), OX40 (anti-OX40 antibodies), RANKL (anti-RANKL antibodies) and GITR (anti-GITR antibodies), epithelial and mesenchymal markers of circulating tumor cells, Prostatic Acid Phosphatase (PAP) (anti-PAP antibodies), prostate-specific antigen (PSA) (anti-PSA antibodies), soluble mesothelin-related peptides (SMRP) (anti-SMRP antibodies), somatostatin receptor (SR) (anti-SR antibodies), Urokinase plasminogen activator (uPA) (anti-uPA antibodies), plasminogen activator inhibitor (PAI-1) (anti-PAI-J antibodies). TCR (e.g., MHC class I or class II molecules) (anti-MHC I/II antibodies), A2a Receptor (anti-A2aR antibodies), MICA family (anti-MICA/B antibodies), RAET1/ULBP family (anti-RAET1/ULBP antibodies), HLA-E (anti-HLA-E antibodies).

In accordance with one or more embodiments, single-domain antibodies of the present application can exhibit binding affinity and specificity towards other antigens associated with cancer cells including but not limited to glioma-associated antigen, carcinoembryonic antigen (CEA), beta-human chorionic gonadotropin, thyroglobulin, RAGE-1, MN-CAIX, RU1, RU2 (AS), intestinal carboxyl esterase, mut hsp70-2, M-CSF, prostase, prostate-specific antigen (PSA), PAP, NY-ESO-1, LAGE-1a, prostein, PSMA, prostate-carcinoma tumor antigen-1 (PCTA-1), MART-1, MAGE, TRP-1, TRP-2 BAGE, GAGE-1, GAGE-2, RAGE, p15, ELF2M, neutrophil elastase, ephrinB2, IGF-I receptor, mesothelin, E2A-PRL, H4-RET, IGH-IGK, MYL-RAR; other protein-based antigens include TSP-180, p185erbB2, p180erbB-3, c-met, nm-23HI, TAG-72, CA 19-9, CA 72-4, CAM 17.1, NuMa, K-ras, beta-Catenin, CDK4, Mum-1, p 15, p 16, 43-9F, 5T4, 791Tgp72, beta-HCG, BCA225, BTAA, CA 15-3CA 27.29BCAA, CA 195, CA 242, CA-50, CAM43, CD68P1, CO-029, G250, Ga733, EpCAM, HTgp-175, M344, MA-50, MG7-Ag, MOV18, NB/70K, NY-CO-1, RCAS 1, SDCCAG16, TA-90, Mac-2 binding protein\cyclophilin C-associated protein, TAAL6, TAG72, TLP, and TPS, and SIRP-alpha receptor.

In accordance with one or more embodiments, single-domain antibodies of the present application can exhibit binding affinity and specificity towards one or more antigens associated with bacteria. For example, the single-domain antibodies can exhibit affinity and specificity towards bacteria-associated antigens including but not limited to polysaccharides or peptide antigens associated with P. aeruginosa, S. aureus, Clostridium difficile, Acinetobacter baumannii, STEC infection, Bacillus anthracis, Yersinia pestis, Francisella tularensis, Coxiella burnetii, and Klebsiella pneumonia among others.

In accordance with one or more embodiments, the single-domain antibodies of the present application can exhibit binding affinity and specificity towards one or more antigens associated with a virus. For example, the single-domain antibodies can exhibit affinity and specificity towards virus-associated antigens including but not limited to Epstein Barr virus antigens EBVA, human papillomavirus (HPV) antigens E6 and E7, coronavirus surface antigens, influenza virus surface antigens, Variola viruses, viral hemorrhagic fevers, and HIV surface antigens among others.

In accordance with one or more embodiments, single-domain antibodies of the present application can exhibit binding affinity and specificity towards one or more antigens associated with a parasite. For example, the single-domain antibodies can exhibit affinity and specificity towards parasite-associated antigens including but not limited to antigen associated with malaria, Leishmaniasis, Chagas Disease, Toxoplasmosis, Schistosomiasis, Cysticercosis, and Strongyloidiasis.

In one or more embodiments, these high-affinity and high-specificity single-domain antibodies of the present application, including single-domain antibodies that exhibits specificity and high affinity toward an antigen associated with a cancer cell, bacteria or virus, (e.g., anti-HER2 VHHs), can be used for flow cytometry, ELISA, immunohistochemistry, cell imaging, and ex-vivo cancer phenotyping methods.

In one or more embodiments, the single-domain antibodies of the present application can be human, humanized or chimeric antibodies. For instance, in one or more embodiments, the single-domain antibodies can be humanized in a manner described in Cécile Vincke et al., THE JOURNAL OF BIOLOGICAL CHEMISTRY VOL. 284, NO. 5, pp. 3273-3284 Jan. 30, 2009, which is hereby incorporated by reference in its entirety. More specifically, in one or more embodiments, the amino acids of the single-domain antibodies can generally be humanized in framework regions 1, 3, and 4 (i.e., outside of the framework-2 region, positions 42, 49, 50, and 52), and have a neutral or minimal effect on the properties of the single-domain antibodies. In contrast, certain framework-2 region humanization substitutions, namely Phe-42→Val and Gly/Ala-52→Trp, can be detrimental for antigen affinity due to a repositioning of the H3 loop, and thus should generally be avoided during humanization. However, it is noted that substitutions Glu-49→Gly and Arg-50→Leu in the framework-2 region can actually increase stability of the autonomous domain, but can result in decreased solubility.

In addition, in one or more embodiments, amino acids of the VHHs and constructs of the present application can be substituted with homologous amino acids (e.g. polar and nonpolar amino acids, hydrophobic and hydrophilic amino acids, positively-charged and negatively charged amino acids, and aromatic amino acids) such that VHHs and constructs have substantially equivalent biological activity. Furthermore, to maintain substantially equivalent biological activity, amino acids within functional domains of the VHHs and the constructs of the present disclosure are preferably conserved.

Single-Domain Antibodies Exhibiting Affinity towards CD16a (Anti-CD16a VHHs)

In accordance with at least one embodiment of the present application, single-domain antibodies (VHHs) are provided that exhibit high affinity and specificity toward CD16a receptors with negligible binding to CD16b-NA1 and CD32b receptors (referred to herein as “anti-CD16a VHHs”). In general, VHHs lack light chains, lack an Fc region, possess high-target specificity, affinity in (sub-) nanomolar range, high stability, small size (˜15 kDa), and low immunogenicity/toxicity, and can be easily produced in both mammalian and E. coli cells (7-9). Based on these unique characteristics, in accordance with one or more embodiments, exemplary VHHs of the present application were produced by immunizing a llama with recombinant CD16a protein (rCD16a). Then, by using phage display, VHH clones were isolated with high affinity and specificity toward CD16a and without cross reactivity with CD16b (e.g., CD16b-NA1) or CD32b. VHHs can be produced using various materials, as exemplified in the example sections below (see Examples Section 1).

In one or more embodiments, the single-domain antibody can comprise at least one of the amino acid sequences of C1 (SEQ ID NO: 14) and E3 (SEQ ID NO: 15), which exhibit affinity and specificity toward CD16a. These amino acid sequences are provided in Table 1 below.

TABLE 1
The amino acid sequences of the C1 (SEQ ID NO: 14) and E3 (SEQ ID NO: 15) anti-
CD16a VHHs with affinity and specificity toward CD16a. The HA-Tag (YPYDVPDYA (SEQ
ID NO: 12)) and histidine tag (HHHHHHHH (SEQ ID NO: 13)) are constructed at VHH C-
terminal, respectively.

| VHH | | | | | | | | |
|---|---|---|---|---|---|---|---|---|
| Code | Framework 1 | CDR1 | Framework 2 | CDR2 | Framework 3 | CDR3 | Framework 4 | Tag |
| C1 | QVQLQESGGG | GRTFRL | MGWFRQA | IKMIDG | LYGDPVKGRFTISRDN | ASVSRVTGS | WGQGTQ | HA- |
| (SEQ | LVQAGGSLRL | YR (SEQ | PGKEREFV | ST (SEQ | TKFMAYLQMNSLKPE | YDS (SEQ ID | VTVSS | Tag + |
| ID | SCAAS (SEQ ID | ID NO: | GS (SEQ ID | ID NO: | DTAVYYC (SEQ ID | NO: 33) | (SEQ ID | Histag |
| NO 14) | NO: 28) | 29) | NO: 30) | 31) | NO: 32) | | NO: 34) | |
| E3 | QVQLQESGGG | GRTFRL | MGWFRQA | IKMIDG | LFGDPVKGRFTISRDN | ASVSRNTGS | WGQGTQ | HA- |
| (SEQ | LVQAGGSARL | YR (SEQ | PGKEREFV | SS (SEQ | TKFMVYLQMNSLKPE | WDS (SEQ ID | VTVSS | Tag + |
| ID | SCAAS (SEQ ID | ID NO: | AS (SEQ ID | ID NO: | DTAVYYC (SEQ ID | NO: 39) | (SEQ ID | Histag |
| NO 15) | NO: 35) | 29) | NO: 36) | 37) | NO: 38) | | NO: 34) | |

In one or more embodiments, anti-CD16a VHHs of the present application comprise an amino acid sequence having at least 85%, 90%, 95%, 98% or 99% sequence identity with the framework regions 1, 2, 3 and/or 4 of SEQ ID NOs: 14-15 while still exhibiting the desired binding and functional properties.

### Single-Domain Antibodies Exhibiting Affinity Toward Cancer Cells, Bacteria, Parasites, or Viruses

In accordance with at least one embodiment of the present application, a single-domain antibody (VHH) is provided that exhibits high affinity and specificity toward antigens associated with cancer cells, bacteria, parasite, or virus. In one or more embodiments, VHHs can be produced by immunization of camelids (e.g., camels, llamas) with the selected antigen associated with the particular type of cancer cell, bacteria, parasite, or virus. Subsequently, using phage display, VHH clones can be isolated with high affinity and specificity toward the selected antigen. Phage display can be performed, for example, as described in Els Pardon, et al., “A general protocol for the generation of Nanobodies for structural biology,” Nat Protoc. 2014 March; 9(3):674-93. doi: 10.1038/nprot.2014.039, and Kristian Daniel Ralph Roth, et al., “Developing Recombinant Antibodies by Phage Display Against Infectious Diseases and Toxins for Diagnostics and Therapy,” Front Cell Infect Microbiol. 2021 Jul. 7; 11:697876. doi: 10.3389/fcimb.2021.697876, which are both hereby incorporated by reference in their respective entireties. VHHs can be produced using various materials, as exemplified in the example sections below (see Examples Section 1).

### Single-Domain Antibodies Exhibiting Affinity Toward HER2 (Anti-HER2 VHHs)

In accordance with one or more embodiments, a single-domain antibody (VHH) of the present application can selectively bind to HER2-expressing cancer cells (referred to herein as “anti-HER2 VHHs”). To address the current unmet needs, single-domain antibodies of the present application have been produced, which exhibit high affinity and specificity towards HER2 with negligible binding to HER1, HER3, and HER4. In accordance with one or more embodiments, anti-HER2 VHHs of the present application were produced by immunizing a llama with recombinant HER2 protein. Then, by using phage display, VHH clones were isolated with high affinity and specificity towards HER2.

For example, in one or more embodiments, the anti-HER2 VHHs of the present application can be utilized in methods for identifying HER2-expressing cancer lesions in a subject via magnetic resonance imaging (MRI), PET/CT imaging, Single photon emission computed tomography (SPECT) or any other radioactive or non-radioactive tracer. Specifically, in accordance with at least one embodiment, a method for identifying HER2-expressing cancer lesions in a subject via MRI and/or PET/CT includes the steps of administering an imaging substance comprising the anti-HER2 VHH to a subject, performing an MRI and/or PET/CT of the subject to produce an image (e.g., MRI and/or PET/CT image, and identifying HER2-expressing cancer lesions in the MRI and/or PET/CT image. In methods in which MRI imaging is utilized, the HER2-expressing cancer cells can be identified in the MRI image using an imaging software, such as the VivoQuant™ software (ASPECT Imaging), for example. In methods in which PET/SPECT/CT imaging is utilized, the HER2-expressing cancer cells can be identified in the PET/SPECT/CT image using an imaging software, such as the PMOD software Albira Imaging System from Bruker, for example.

In one or more embodiments, the anti-HER2 VHHs facilitate recognition of various types of HER2-expressing cancer cells, including HER2-expressing ovarian cancer cells (e.g., HER2-expressing ovarian cancer cells from a metastatic lesion), breast cancer cells, cervical cancer cells, bladder cancer cells, gallbladder cancer cells, testicular cancer cells, lung cancer, gastric cancer, esophageal cancer, and/or uterine cancer cells.

In one or more embodiments, the anti-HER2 VHHs can be produced using various materials, as exemplified in the example sections below (see Examples Section 2). In one or more embodiments, the anti-HER2 VHHs can comprise at least one of the amino acid sequences of Table 2 below.

TABLE 2
The amino acid sequences of the selected anti-HER2 VHHs. The c-myc
(GSEQKLISEEDL (SEQ ID NO: 1)) and histidine (HHHHHHHHHHHH (SEQ ID NO: 2))
tags are constructed at VHH C-terminal, respectively.

| Anti- | | | | | | | | |
|---|---|---|---|---|---|---|---|---|
| HER2 | | | | | | | | |
| VHH | | | | | | | Framework | |
| Codes | Framework 1 | CDR1 | Framework 2 | CDR2 | Framework 3 | CDR3 | 4 | Tag |
| A1 | QVQLQESGG | GHTLSTYV | VAWFRQAPG | IGWSSRSP | YYPDSVKGRFTISEDN | AADSYPRRW | WGQGTQVTV | C-Myc + |
| (SEQ ID | GLVQPGGSL | (SEQ ID | KERESVAA | (SEQ ID | TKNTVYLQMNSLKPD | DRESDFGS | SS (SEQ | Histag |
| \|NO 11) | RLSCAAS | NO: 41) | (SEQ ID NO: | NO: 43) | DTAVYYC (SEQ ID | (SEQ ID | ID NO: 34) | |
| | (SEQ ID NO: | | 42) | | NO: 44) | NO: 45) | | |
| | 40) | | | | | | | |
| A3 | QVQLQESGG | GRTSDLFP | VGWFRQAP | IRWSGGVI | GYGDPVKGRFTISTD | AAGPREST | WGQGTHVTVS | C-Myc + |
| (SEQ ID | GLVQAGGSL | (SEQ ID | GKEREIVAA | (SEQ ID | NAKNTVYLQMNSLK | VTWNY | S (SEQ | Histag |
| NO 3) | RISCAAS | NO: 47) | (SEQ ID NO: | NO: 49) | PEDTAVYYC (SEQ ID | (SEQ ID | ID NO: 52) | |
| | (SEQ ID NO: | | 48) | | NO: 50) | NO: 51) | | |
| | 46) | | | | | | | |
| C9 | QVQLQESGG | GRTVSRYS | MGWFRQAP | IAWFGGWP | YYADSVKGRFTISRDN | AARQWART | WGQGTQVTV | C-Myc + |
| (SEQ ID | GLVQAGGSL | (SEQ ID | GKEREFVAA | (SEQ ID | AKNTVYLQMNSLKPE | WDY | SS (SEQ | Histag |
| NO 4) | RLSCAAS | NO: 53) | (SEQ ID NO: | NO: 55) | DTAVYYC (SEQ ID | (SEQ ID | ID NO: 34) | |
| | (SEQ ID NO: | | 54) | | NO: 56) | NO: 57) | | |
| | 28) | | | | | | | |
| D6 | QVQLQESGG | GRTFSTYV | VGWFRQAP | IGWASNSP | YYADSVKGRFTISRDN | AADSYPRRW | WGQGTQVTV | C-Myc + |
| (SEQ ID | GLVQAGGSL | (SEQ ID | GKERESVAA | (SEQ ID | TKNTVYLQMKSLKPD | DRESDFGS | SS (SEQ | Histag |
| NO 5) | TLSCAAS | NO: 59) | (SEQ ID NO: | NO: 61) | DTAVYYC (SEQ ID | (SEQ ID | ID NO: 34) | |
| | (SEQ ID NO: | | 60) | | NO: 62) | NO: 45) | | |
| | 58) | | | | | | | |
| E5 | QVQLQESGG | GRTLSSYV | VGWFRQAP | IGWSRTST | FYTDSVKGRFTISRDN | AADSSPRRW | WGQGTQVTV | C-Myc + |
| (SEQ ID | GLVQAGGSL | (SEQ ID | GKEREVVAA | (SEQ ID | TENTVYLQMNSLKPG | DRESDFGS | SS (SEQ | Histag |
| NO 6) | RLDCAAS | NO: 64) | (SEQ ID NO: | NO: 66) | DTAVYYC (SEQ ID | (SEQ ID | ID NO: 34) | |
| | (SEQ ID NO: | | 65) | | NO: 67) | NO: 68) | | |
| | 63) | | | | | | | |
| E9 | QVQLQESGG | GRTLSAYV | VGWFRQAP | IGWSTHSV | YYADSVKGRFTISRDN | AADSSPRRW | WGQGTQVTV | C-Myc + |
| (SEQ ID | GSVQAGGSL | (SEQ ID | GRERESVAA | (SEQ ID | TENTVYLQMNSLKPD | DRESDFGS | SS (SEQ | Histag |
| NO 7) | KLSCAAS | NO: 70) | (SEQ ID NO: | NO: 72) | DTAVYYC (SEQ ID | (SEQ ID | ID NO: 34) | |
| | (SEQ ID NO: | | 71) | | NO: 73) | NO: 68) | | |
| | 69) | | | | | | | |
| F2 | QVQLQESGG | GRTLSAYV | VGWFRQAP | IGWSTHSV | YYADSVKGRFTISRDN | AADSSPRRW | WGQGTQVTV | C-Myc + |
| (SEQ ID | GLVQAGGSL | (SEQ ID | GRERESVAA | (SEQ ID | TENTVYLQMNSLKPD | DRESDFGS | SS (SEQ | Histag |
| NO 8) | KLSCAAS | NO: 70) | (SEQ ID NO: | NO: 72) | DTAVYYC (SEQ ID | (SEQ ID | ID NO: 34) | |
| | (SEQ ID NO: | | 71) | | NO: 73) | NO: 68) | | |
| | 74) | | | | | | | |
| F4 | QVQLQESGG | GRTLSAYV | VGWFRQAP | IGWSSRSP | YYPDSVKGRFTISEDN | AADSYPRRW | WGQGTQVTV | C-Myc + |
| (SEQ ID | GLVQAGGSL | (SEQ ID | GKERESVAA | (SEQ ID | TKNTVYLQMNSLKPD | DRESDFGS | SS (SEQ | Histag |
| NO 9) | KLSCAAS | NO: 70) | (SEQ ID NO: | NO: 43) | DTAVYYC (SEQ ID | (SEQ ID | ID NO: 34) | |
| | (SEQ ID NO: | | 60) | | NO: 44) | NO: 45) | | |
| | 74) | | | | | | | |
| H7 | QVQLQESGG | GHTLSTYV | VAWFRQAPG | IGWSTRSP | YYADSVKGRFTISEDN | AADSYPRRW | WGQGTQVTV | C-Myc + |
| (SEQ ID | GLVQAGGSL | (SEQ ID | KERESVAA | (SEQ ID | TKNTVYLQMNSLKPD | DRESDFGS | SS (SEQ | Histag |
| NO 10) | RLSCAAS | NO: 41) | (SEQ ID NO: | NO: 75) | DTAVYYC (SEQ ID | (SEQ ID | ID NO: 34) | |
| | (SEQ ID NO: | | 42 | | NO: 76) | NO: 45) | | |
| | 28) | | | | | | | |

In one or more embodiments, anti-HER2 VHHs of the present application comprise an amino acid sequence having at least 85%, 90%, 95%, 98% or 99% sequence identity with the framework regions 1, 2, 3 and/or 4 of SEQ ID NOs: 3-11 while still exhibiting the desired binding and functional properties.

### Constructs Having Single-Domain Antibodies

In accordance with one or more embodiments of the present application, constructs are provided that comprise two or more single-domain antibodies of the present application. In one or more embodiments, the construct comprises an anti-CD16a single-domain antibody as described above (“anti-CD16a VHH”) and at least one other single-domain antibody (“anti-antigen VHHs”) that exhibits binding affinity and specificity towards an antigen associated with a cancer cell, bacteria, parasite, or virus.

More specifically, in one or more embodiments, the construct includes a first single-domain antibody having an amino acid sequence that exhibits specificity and high affinity towards the CD16a receptor on the surface of natural killer (NK) cells without cross reactivity with CD16b (e.g., CD16b-NA1) or CD32b. In one or more embodiments, the amino acid sequence that exhibits specificity and high affinity towards the CD16a receptor without cross reactivity with CD16b (e.g., CD16b-NA1) or CD32b is SEQ ID NO: 14. In at least one embodiment, the amino acid sequence that exhibits specificity and high affinity towards the CD16a receptor without cross reactivity with CD16b (e.g., CD16b-NA1) or CD32b is SEQ ID NO: 15. The construct also includes at least a second single-domain antibody having an amino acid sequence that exhibits affinity and specificity toward an antigen associated with a cancer cell, bacteria, or virus (anti-antigen VHH). The first and second single-domain antibodies are fused with each other with or without a linker. In embodiments in which the single-domain antibodies are fused together via a linker, the linker can be a human muscle aldolase (HMA) linker for example. An exemplary HMA linker is shown in Table 3 below. In at least one embodiment, the linker can be a (GGGSG)n-based linker (n=1, 2, 3, 4, . . . ), (SEQ ID NO: 82) or any other flexible, semi-flexible, or rigid linker. In one or more embodiments in which the first single-domain antibody and additional single-domain antibodies are fused without a linker, the single-domains antibodies can be recombinantly fused in tandem one after another.

A schematic representation of an exemplary construct featuring a first single-domain antibody (anti-CD16a VHH) and a second single-domain antibody (anti-antigen VHH) joined by a linker is shown in FIGS. 1B-1C. As shown in FIG. 1C, the anti-CD16a VHH binds to the CD16a receptor on the surface of the NK cell and the anti-antigen VHH binds to a surface antigen on the surface of the target (e.g., cancer cell, bacterial cell, virus). Accordingly, the construct not only activates NK cells via binding of the anti-CD16a VHH, but also facilitates recognition of target cancer cells by NK cells via the binding of the anti-antigen VHH to the surface antigen of the target.

In accordance with one or more embodiments, the construct can comprise multiple anti-CD16a VHHs fused to each other and fused to another single-domain antibody having an amino acid sequence that exhibits affinity and specificity toward an antigen associated with a cancer cell, bacteria or virus. The construct with multiple CD16a VHHs can activate one or more NK cells via binding to facilitate recognition of the target (e.g., cancer cell, bacterial cell, parasite, virus) by the NK cells. FIG. 1D is a schematic representation of various configurations of constructs having one or more anti-CD16a VHHs bound to one or more anti-antigen VHHs via a linker.

In some embodiments, two identical VHHs are fused to each other, one of which is further fused to a different VHH. In some embodiments, two different VHHs are fused to each other, which can optionally be further fused to one or more other VHHs. For instance, in a construct comprising two fused VHHs, one VHH can have affinity and specificity to a first immune cell while the other has affinity and specificity to a second immune cell. Likewise, in one or more embodiments, a first VHH of a construct can have affinity and specificity to a first tumor cell while a second VHH has affinity and specificity to a second tumor cell. Moreover, in one or more embodiments, a first VHH of a construct can have affinity and specificity to an immune cell and a second VHH of a construct can have affinity and specificity to a microorganism, an infected cell, a tumor cell, an inflamed cell, an apoptotic cell, or a foreign cell, without limitation. In one or more embodiments, as exemplified in FIG. 1D, one or more anti-CD16a VHHs can be bound to one or more VHHs having affinity and specificity to a microorganism, an infected cell, a tumor cell, an inflamed cell, an apoptotic cell, or a foreign cell (“anti-antigen VHHs”), where the one or more anti-antigen is a VHH, scFv, mAb, or any other form of antibody. Also as exemplified in FIG. 1D, in one or more embodiments, two or more anti-CD16a VHHs can be engineered in tandem to recognize two or more different epitopes on CD16a antigen. Similarly, two or more anti-antigen VHHs can be engineered in tandem to recognize two or more different epitopes on a target antigen. VHHs can be specific for the same or different antigens related to a microorganism, an infected cell, a tumor cell, an inflamed cell, an apoptotic cell, or a foreign cell.

In one or more embodiments, the VHHs constructs can be produced in a manner as described in Ulrich Brinkmann & Roland E. Kontermann (2017), “The making of bispecific antibodies,” mAbs, 9:2, pp. 182-212, DOI: 10.1080/19420862.2016.1268307, which is hereby incorporated by reference in its entirety. For instance, in one or more embodiments, the domains of two or more single-domain antibodies can be fused to make the construct molecule, which can be a bivalent, trivalent, or multivalent molecule with one or more specificities (i.e., monospecific, bispecific, trispecific, tetraspecific, multispecific). The terms “bivalent”, “trivalent”, “multivalent” denote the presence of two binding sites, three binding sites, and multiple binding sites, respectively, in an antigen binding antibody molecule.

For example, in one or more embodiments, two VHH domains can be fused with a long hinge sequence derived from the upper hinge of a llama IgG2a to form a bispecific construct. A flexible linker, such as a linker from a shark immunoglobulin new antigen receptors (VNAR) can be used to combine the two variable domains. In exemplary embodiment, the linker can comprise a native shark IgNAR hinge (PGVQPSP (SEQ ID NO: 16)) followed by a flexible GGGGSG (SEQ ID NO: 17) sequence. Other possible linkers include flexible (G3S) 4 linkers (SEQ ID NO: 27), flexible GGGGSGGGS (SEQ ID NO: 18) linkers, an HMA linker (see Table 3 below), a (GGGSG) n-based linker (n=1, 2, 3, . . . ) (SEQ ID NO: 83) (GGGSG (SEQ ID NO: 19), or any other flexible, semi-flexible, or rigid linker.

In at least one embodiment, the construct can comprise two VHHs—an anti-CD16a VHH and an anti-HER2 VHH—fused with each other with or without a linker to create a Bispecific Killer Cell Engager (“BiKE”, specifically, a “BiKE: HER2/CD16a”) with specificity and affinity towards the CD16a receptor and HER2, without cross reactivity with CD16b (e.g., CD16b-NA1) or CD32b. As such, the BiKE: HER2/CD16a binds to the CD16a receptor on the surface of the NK cell and HER2-expressing cancer cells, thereby activating NK cells to facilitate recognition of target HER2+ cancer cells by NK cells.

While examples in the present application that utilize a “BiKE” generally refer to BIKE: HER2/CD16a constructs, it should be understood that in accordance with other embodiments of the present application, a “BiKE” construct can generally include an anti-CD16a VHH and another single-domain antibody (anti-antigen VHH) having an amino acid sequence that exhibits affinity and specificity toward a different antigen associated with a cancer cell, bacteria, or virus (FIG. 1C).

In one or more embodiments, the single-domain antibodies or the constructs comprising the single-domain antibodies may be administered alone or in combination with other types of treatments, such as cancer medications, antibiotics, anti-parasitic, and anti-viral medications. The single-domain antibodies or the single-domain antibody constructs of the present application can be administered concurrently or in tandem with the other types of treatments.

In one or more embodiments, the single-domain antibodies or the constructs comprising the single-domain antibodies can be administered in combination with large molecule drugs (biologics) and/or small molecule drugs and/or cell-based therapeutics for the treatment of cancer. Examples of such drugs include but are not limited to anti-HER2 antibodies (Trastuzumab, Pertuzumab, Enhertu, etc.), anti-ER antibodies, anti-PR antibodies, anti-PDL-1 antibodies, anti-PD-1 antibodies (e.g., Keytruda), anti-CTL4 antibodies, anti-CD47 antibodies, anti-CD19 antibodies, cisplatin, paclitaxel, irinotecan, 5-FU, Tisagenlecleucel, Axicabtagene ciloleucel, Brexucabtagene autoleucel, and Lisocabtagene maraleucel, among others.

In one or more embodiments, the single-domain antibodies or the constructs comprising the single-domain antibodies may be administered in combination with large molecule drugs (biologics) and/or small molecule drugs and/or cell-based therapeutics for the treatment of bacterial infections. Examples of such drugs includes but not limited to Bezlotoxumab, Raxibacumab, Obiltoxaximab, Suvratoxumab, and commonly used antibiotics (e.g., penicillin group, macrolides, cephalosporin group, etc).

In one or more embodiments, the single-domain antibodies or the constructs comprising the single-domain antibodies may be administered in combination with large molecule drugs (biologics) and/or small molecule drugs and/or cell-based therapeutics for the treatment of parasitic infections. Examples of such drugs include but are not limited to metronidazole, tinidazole, and ivermectin among others.

In one or more embodiments, the single-domain antibodies or the constructs comprising the single-domain antibodies may be administered in combination with large molecule drugs (biologics) and/or small molecule drugs and/or cell-based therapeutics for the treatment of viral infections. Examples of such drugs include but are not limited to remdesivir, baricitinib, palivizumab, zanamivir, peramivir, oseltamivir, and baloxavir marboxil among others.

The single-domain antibody or construct is substantially purified (e.g., substantially free from substances that limit its effect or produce undesired side-effects). Various delivery systems are known and can be surface-decorated with an antibody of the present application, including liposomes, microparticles, microcapsules, engineered cells, viruses, or other vectors capable of expressing the antibody, (see, e.g., Wu, et al., J Biol Chem 262:4429 (1987)).

The single-domain antibody or construct body can be administered to the mammal in any acceptable manner. Methods of introduction include but are not limited to parenteral, subcutaneous, intraperitoneal, intrapulmonary, intranasal, epidural, inhalation, and oral routes, and if desired for immunosuppressive treatment, intralesional administration. Parenteral infusions include intramuscular, intradermal, intravenous, intraarterial, or intraperitoneal administration. The single-domain antibody or construct or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the therapeutic single-domain antibody or construct or compositions of the present application into the central nervous system by any suitable route, including intraventricular and intrathecal injection: intraventricular injection may be facilitated by an intraventricular catheter, for example, attached to a reservoir, such as an Ommaya reservoir. In addition, the single-domain antibody or construct is suitably administered by pulse infusion, particularly with declining doses of the single-domain antibody or construct. Preferably the dosing is given by injections, most preferably intravenous or subcutaneous injections, depending in part on whether the administration is brief or chronic.

Pulmonary administration can also be employed, e.g., by use of an inhaler or nebulizer, and formulation with an aerosolizing agent. The single-domain antibody or construct may also be administered into the lungs of a patient in the form of a dry powder composition (see, e.g., U.S. Pat. No. 6,514,496).

In a specific embodiment, it may be desirable to administer the therapeutic single-domain antibody or construct or compositions of the present application locally to the area in need of treatment; this may be achieved by, for example, and not by way of limitation, local infusion, topical application, by injection, by means of a catheter, by means of a suppository, or by means of an implant, said implant being of a porous, non-porous, or gelatinous material, including membranes, such as silastic membranes, or fibers. Preferably, when administering a single-domain antibody or construct of the present application, care must be taken to use materials to which the protein does not absorb.

In another embodiment, the single-domain antibody or construct can be delivered in a vesicle, in particular a liposome (see Langer, Science 249:1527 (1990); Treat, et al., in Liposomes in the Therapy of Infectious Disease and Cancer, Lopez-Berestein, et al., eds., pp. 353-365 (1989); Lopez-Berestein, ibid., pp. 317-27; see generally ibid.).

In yet another embodiment, the single-domain antibody or construct can be delivered in a controlled release system. In one embodiment, a pump may be used (see Langer, Science 249:1527 (1990); Sefton, CRC Crit Ref Biomed Eng 14:201 (1987); Buchwald, et al., Surgery 88:507 (1980); Saudek, et al., N Engl J Med 321:574 (1989)). In another embodiment, polymeric materials can be used (see Medical Applications of Controlled Release, Langer, et al., eds., CRC Press (1974); Controlled Drug Bioavailability, Drug Product Design and Performance, Smolen, et al., eds., Wiley (1984); Ranger, et al., J Macromol Sci Rev Macromol Chem 23:61 (1983); see also, Levy, et al., Science 228:190 (1985); During, et al., Ann Neurol 25:351 (1989); Howard, et al., J Neurosurg 71:105 (1989)). In yet another embodiment, a controlled release system can be placed in proximity of the therapeutic target.

The present application also provides pharmaceutical compositions. Such compositions comprise a therapeutically effective amount of the single-domain antibody or construct and a physiologically acceptable carrier. In a specific embodiment, the term “physiologically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such physiological carriers can be sterile liquids, such as water and oils, including those of petroleum, animal, vegetable, or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable carriers are described in “Remington's Pharmaceutical Sciences” by E. W. Martin. Such compositions will contain an effective amount of the antibody, preferably in purified form, together with a suitable amount of carrier so as to provide the form for proper administration to the patient. The formulation should suit the mode of administration.

In at least one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In one or more embodiments, the single-domain antibodies (VHHs) and constructs of the present application can be conjugated to prodrugs, peptides, proteins, enzymes, viruses, lipids, biological response modifiers, therapeutic agents, pharmaceutical agents, or PEG. For instance, the single-domain antibodies can be conjugated or fused to a therapeutic agent, which can include but are not limited to, detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, or a combination thereof. The single-domain antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of suitable chemiluminescent labeling compounds include but are not limited to luminol, isoluminol, imidazole, acridinium salt, theromatic acridinium ester, and oxalate ester.

The present application also provides a pharmaceutical pack or kit comprising one or more containers filled with one or more of the ingredients of the pharmaceutical compositions of the present application. Optionally associated with such container(s) can be a notice in the form prescribed by a governmental agency regulating the manufacture, use or sale of pharmaceuticals or biological products, which notice reflects approval by the agency of manufacture, use or sale for human administration.

Further provided in one aspect of the present application is a method of treating a disease in an individual, comprising administering to the individual an effective amount of the pharmaceutical compositions comprising single-domain antibodies or constructs of the present application as described above. In some embodiments, the disease is a cancer. In some embodiments, the cancer is breast cancer, ovarian cancer, ovarian carcinoma, renal cancer, melanoma, head and neck cancer, lung cancer, glioblastoma, prostate cancer, bladder carcinoma, or lymphoma. In some embodiments, the disease is a respiratory disease, an inflammatory disease, or an autoimmune disease. In some embodiments, the disease is an infectious disease caused by a microorganism, such as a virus including RNA and DNA viruses, a Gram-positive bacterium, a Gram-negative bacterium, a protozoa or a fungus.

Constructs Comprising Single-Domain Antibodies that Exhibit Affinity towards CD16a and HER2

In accordance with one or more embodiments, the bispecific single-domain antibody construct of the present application can selectively bind to CD16a on NK cells and HER2 on HER2-expressing cancer cells (referred to herein as “BiKE: HER2/CD16a”). Thus, in one or more embodiments, two VHHs can be fused with each other with or without a linker to create a BiKE with specificity and affinity towards the CD16a receptor and HER2, without cross reactivity with CD16b (e.g., CD16b-NA1) or CD32b. In one or more embodiments, the linker can be an HMA linker, as shown in Table 3 below, for example. In one or more embodiments, the linker can be a (GGGSG) n-based linker (n=1, 2, 3, . . . ), (SEQ ID NO: 83) or any other flexible, semi-flexible, or rigid linker.

In one or more embodiments, the BIKE: HER2/CD16a of the present application can be utilized to engage the NK cells and kill the HER2+ cancer cells. BiKEs with specificity and affinity towards the CD16a receptor and HER2 can address aforementioned challenges and other problems related to effective killing of HER2-expressing cancer cells. In one or more embodiments, the BiKE: HER2/CD16a can comprise at least one of the amino acid sequences of Table 1 and at least one of the amino acid sequences of Table 2. An example is shown below in Table 3.

TABLE 3
The amino acid sequence of the engineered BiKE:HER2/CD16a
(SEQ ID NO: 20) by fusing E5 anti-HER2 VHH with C1 anti-CD16a
VHH via a HMA linker. anti-HER2 VHHs (see Table 18). The c-myc
(GSEQKLISEEDL (SEQ ID NO: 1)) and histidine (HHHHHHHHHHHH
(SEQ ID NO: 2)) tags are constructed at C-terminal, respectively.

| Name | Amino Acid Sequence |
|---|---|
| E5C1 BIKE | QVQLQESGGGLVQAGGSLRLDCAASGRTLSSYVVGWFRQAP |
| | GKEREVVAAIGWSRTSTFYTDSVKGRFTISRDNTENTVYLQM |
| | NSLKPGDTAVYYCAADSSPRRWDRESDFGSWGQGTQVTVSS |
| | PSGQAGAAASESLFVSNHASQVQLQESGGGLVQAGGSLRLS |
| | CAASGRTFRLYRMGWFRQAPGKEREFVGSIKMIDGSTLYGDP |
| | VKGRFTISRDNTKFMAYLQMNSLKPEDTAVYYCASVSRVTG |
| | SYDSWGQGTQVTVSSWELQGSEQKLISEEDLHHHHHHHHHH |
| | HH* |
| | (SEQ ID NO: 20) |

*E5 HER2 VHH - HMA Linker (BOLD)- C1 CD16 VHH - Protease Site (italics) - cMyc Tag - Histag

### Assays and Methods of Use of Single-Domain Antibodies

In one or more embodiments, the single-domain antibodies (VHHs) of the present application can be used for various physicochemical and biological applications.

For example, in one or more embodiments, the single-domain antibodies can be used for enzyme-linked immunosorbent assays (ELISA). For instance, in at least one embodiment of the present application, an ELISA method is performed in which a sample comprising one or more antigens (e.g., HER2 or CD16a) is immobilized on a solid support. Single-domain antibodies of the present application are then applied over the surface of the sample as the primary antibody, so it can bind the antigen(s). A secondary antibody (e.g., anti-histag or anti-HAtag antibody) that is linked to an enzyme such as HRP (horseradish peroxidase) can be used to recognize the antigen-bound VHH. In a final step, a substance containing the enzyme's substrate is added. If there is binding by the single-domain antibodies to the antigen(s), the subsequent reaction produces a detectable signal in the same (e.g., a color change).

In one or more embodiments, the single-domain antibodies of the present application can be used in flow cytometry methods. In one or more embodiments, flow cytometry methods can be used with the present single-domain antibodies as primary antibodies to determine the presence of one or more antigens on the surfaces of cells. For example, in at least one embodiment, flow cytometry methods can be used with the present single-domain antibodies as primary antibodies to specifically detect HER2 or CD16a proteins on the surface of cancer cells.

Specifically, in accordance with one or more embodiments, a flow cytometry method is performed in which a sample containing cancer cells and anti-HER2 VHHs (or NK cells and anti-CD16a VHHs) is suspended in a fluid and injected into a flow cytometer instrument. A flow cytometry analyzer then provides quantifiable data from the sample, such as whether the cancer cells are HER2+ cancer cells or NK cells are CD16a+ NK cells. A sample of cancer cells suspected of being HER2+ cancer cells can be included in a sample, and anti-HER2 single-domain antibodies of the present application can be applied to bind to HER2 antigen on the surface of the cells. Similarly, a sample of NK cells suspected of being CD16a+ NK cells can be included in a sample, and anti-CD16a single-domain antibodies of the present application can be applied to bind to CD16a antigen on the surface of the cells. Next, a secondary antibody (e.g., anti-histag antibody) linked to a fluorescent probe can be used to bind to the single-domain antibody. The sample can then be analyzed by a flow cytometer to detect HER2 or CD16a expression. In other words, the anti-HER2 VHHs in the sample are used to measure the HER2 expression on the surface of cancer cells.

The same flow cytometry method described above can be used to detect antigens on the surfaces of other targets, such as bacteria, viruses, or other types of cancer cells, by substituting another target (bacteria, viruses, or other types of cancer cells) for the suspected HER2+ cancers cells in the sample, and utilizing a single-domain antibody specific for the other target instead of the anti-HER2 VHH. Other exemplary methods for flow cytometry for the VHHs of the present application are provided in the Examples sections.

In one or more embodiments, the single-domain antibodies (VHHs) of the present application are used in immunohistochemistry applications, such as immunohistochemistry methods for identifying HER2+ cancer cells in tissues. In one or more embodiments, the VHHs of the present applications are also used in methods for ex-vivo cancer phenotyping using histopathology. For example, in ex-vivo cancer phenotyping by immunohistochemistry, suspected tumor tissues can be cryosectioned and fixed on tissue slides. Then, the tissue sections can be stained with anti-HER2 VHH, followed by application of a fluorescently-labeled secondary antibody (e.g., anti-histag or anti-HAtag). Photomicrography can be conducted by using a microscope (e.g., Leica) to detect presence or absence of HER2 expression in tissue sections. The present method of ex-vivo cancer phenotyping can also be performed for other types of cancers as well by utilizing single-domain antibodies specific for other cancer antigens (e.g., EGFR, VEGFR).

In one or more embodiments, the VHHs of the present application are used in cell imaging applications. For example, in one or more embodiments, confocal or fluorescent microscopy can be used with the present single-domain antibodies as primary antibodies to determine the presence of antigens on the surfaces of targets cells or other targets (e.g., viruses). For instance, in one or more embodiments, microscopy methods can be used with the anti-HER2 single-domain antibodies of the present application as primary antibodies to bind to HER2 proteins on the surface of cancer cells. Next, a secondary antibody (e.g., anti-histag antibody) linked to a fluorescent probe can be used to bind to the single-domain antibody. The sample can then be studied by a confocal or fluorescent microscope to detect HER2 expression and examine internalization. In other words, the anti-HER2 VHHs in the sample are used to measure the HER2 expression on the surface of cancer cells. The same microscopy methods can be performed to detect antigens on the surfaces of other targets, such as bacteria, viruses, or other types of cancer cells, by substituting other samples (bacteria, viruses, or other types of cancer cells) for the suspected HER2+ cancers cell sample, and utilizing a single-domain antibody specific for another target instead of the anti-HER2 VHH.

In one or more embodiments, single-domain antibodies of the present application (e.g., anti-HER2 VHHs) are used in in vivo cancer phenotyping applications for identifying different types of cancer lesions in a subject via magnetic resonance imaging (MRI), PET/SPECT/CT imaging, or any other radioactive or non-radioactive tracer. For example, in accordance with at least one embodiment, a method for identifying HER2-expressing cancer lesions in a subject via MRI and/or PET/SPECT/CT includes the steps of administering an imaging substance that is conjugated to the anti-HER2 VHH to a subject, performing an MRI and/or PET/SPECT/CT of the subject to produce an image (e.g., MRI and/or PET/SPECT/CT image), and identifying HER2-expressing cancer lesions in the MRI and/or PET/SPECT/CT scan. The present method of in vivo cancer phenotyping can also be performed for other types of cancers as well by utilizing single-domain antibodies specific for other cancer antigens (e.g., EGFR, VEGFR).

In at least one embodiment, the anti-CD16a VHHs of the present application are used in in vivo NK cell tracking methods. In one or more embodiments, the labeled anti-CD16a VHHs of the present application are used in in vivo NK cell tracking in a subject via magnetic resonance imaging (MRI), PET/SPECT/CT imaging, or any other radioactive or non-radioactive tracer. For example, a method for tracking allogenic NK cells or autologous NK cells in a subject via MRI and/or PET/SPECT/CT includes the steps of administering an imaging substance that is conjugated to the anti-CD16a VHH to a subject, performing an MRI and/or PET/CT of the subject to produce an image (e.g., MRI and/or PET/SPECT/CT image), and identifying the anatomical location of NK cells in the MRI and/or PET/CT scan. In one or more embodiments, the above in vivo cell tracking method can also be utilized to track CD16a+ T cells, macrophages, monocytes, mast cells, and basophils.

In one or more embodiments, a construct for treating HER2-positive cancers is also provided. The construct can exhibit significant anticancer activity towards HER2-positive cancers, and thus can be used as part of an immunotherapy regimen. In at least one embodiment, the construct can include one or more anti-HER2 single-domain antibodies (VHH) in fusion with one or more anti-CD16a VHHs of the present application to engage the NK cells and facilitating the killing of HER2+ cancer cells.

Additional features and aspects of the VHHs (single-domain antibodies) and the constructs comprising the VHHs of the present application and associated methods are further described in the Examples section below. It should be understood that the embodiments described in the Examples are only illustrative and do not limit the scope of the invention.

### Examples—Section 1

### Materials and Methods

Below are materials used for the generation, isolation, and characterization of VHHs used in the following experiments. The list of cells, antibodies and reagents used in this study are listed in Table 4.

TABLE 4
The list of materials used to generate and characterize anti-HER2 nanobody.

| Item | Vendor | Catalogue No. |
|---|---|---|
| Ni-NTA Agarose | Qiagen (MD, USA) | 30230 |
| FreeStyle ™ 293-F Cells | Gibco ™ (NY, USA) | R79007 |
| FreeStyle ™ 293 Expression Medium | Gibco ™ (NY, USA) | 12338018 |
| Anti-Clumping Agent (1000X) | Gibco ™ (NY, USA) | 0010057AE |
| Trypan Blue Solution, 0.4% | Gibco ™ (NY, USA) | 15250061 |
| Fetal Bovine Serum | Gibco ™ (NY, USA) | 10437028 |
| QIAprep Spin Miniprep Kit | Qiagen (MD, USA) | 27106 |
| QIAquick PCR Purification Kit | Qiagen (MD, USA) | 28106 |
| QIAquick Gel Extraction Kit | Qiagen (MD, USA) | 28704 |
| RNase-Free DNase Set | Qiagen (MD, USA) | 79254 |
| RNeasy Mini Kit | Qiagen (MD, USA) | 74104 |
| QIAGEN Plasmid Mega Kit | Qiagen (MD, USA) | 12181 |
| SuperScript ™ IV First-Strand Synthesis | Thermo Scientific ™ (MA, USA) | 18091050 |
| System | | |
| SYBR ™ Safe DNA Gel Stain | Thermo Scientific ™ (MA, USA) | S33102 |
| UltraPure ™ DNA Typing GradeTM TAE | Thermo Scientific ™ (MA, USA) | 24710030 |
| Buffer | | |
| RIPA Lysis and Extraction Buffer | Thermo Scientific ™ (MA, USA) | 89901 |
| DPBS, no calcium, no magnesium | Thermo Scientific ™ (MA, USA) | 14190250 |
| AEBSF Protease Inhibitor | Thermo Scientific ™ (MA, USA) | 78431 |
| Nuclease-Free Water (not DEPC-Treated) | Thermo Scientific ™ (MA, USA) | AM9937 |
| MicroAmp ™ Fast Reaction Tube with Cap | Thermo Scientific ™ (MA, USA) | 4358297 |
| Abgene ™ 96 Well Deepwell Storage Plate | Thermo Scientific ™ (MA, USA) | AB0932 |
| Ventilating Adhesive Plate Seals | Thermo Scientific ™ (MA, USA) | AB0718 |
| S.O.C. Medium | Invitrogen ™ (MA, USA) | 15544034 |
| Polyethylenimine, Linear, MW 25000 | PolyScience (PA, USA) | 23966-1 |
| Carbenicillin (Disodium) | GoldBio (MO, USA) | C-103-25 |
| Isopropyl β-d-1-thiogalactopyranoside (IPTG) | Teknova (CA, USA) | I3325 |
| Histopaque ®-1077 (Ficoll) | Sigma-Aldrich (MA, USA) | 10771-500ML |
| Trypsin-EDTA solution | MiliporeSigma (MA, USA) | T4049-100ML |
| D-(+)-Glucose | MiliporeSigma (MA, USA) | G8270-1KG |
| VCSM13 Interference-Resistant Helper Phage | Integrated Science (VA, USA) | 200251 |
| Gene Pulser ®/MicroPulser ™ Electroporation | Bio-Rad (PA, USA) | 1652083 |
| Cuvettes, 0.1 cm gap | | |
| Precision Plus Protein ™ Dual Color Standards | Bio-Rad (PA, USA) | 1610374 |
| ELISA Coating Buffer | Bio-Rad (PA, USA) | BUF030C |
| Tissue-Plus ™ O.C.T compound | Thermo Scientific ™ (MA, USA) | 23-730-571 |
| Sucrose | Sigma-Aldrich (MA, USA) | S7903-1KG |
| Ethylenediaminetetraacetic acid disodium salt | Sigma-Aldrich (MA, USA) | E5134-100G |
| dihydrate | | |
| 1-Step ™ Turbo TMB-ELISA Substrate | Thermo Scientific ™ (MA, USA) | 34022 |
| Solution | | |
| Escherichia coli (Migula) WK6 | ATCC (VA, USA) | ATCC 47078 |
| Terrific Broth | IBI Scientific (IA, USA) | IB49141 |
| LB Miller Broth | IBI Scientific (IA, USA) | IB49030 |
| Miller's LB Agar | IBI Scientific (IA, USA) | IB49100 |
| Nunc MaxiSorp ™ flat-bottom | Invitrogen ™ (MA, USA) | 44-2404-21 |
| Milk powder, non-fat (skimmed milk), Powder | VWR (PA, USA) | 97063-958 |
| WST-1 Cell Proliferation Reagent | Abcam (MA, USA) | 11644807001 |
| Protein LoBind tubes, PCR clean | Eppendorf (MA, USA) | 0030108302 |
| Octet ® Streptavidin (SA) Biosensor | Sartorius (PA, USA) | 18-5019 |
| Nunc ™ Lab-Tek ™ II Chamber Slide ™ | Thermo Scientific ™ (MA, USA) | 154526PK |
| System | | |
| Pierce ™ BCA Protein Assay Kit | Thermo Scientific ™ (MA, USA) | 23225 |
| SapI restriction enzyme | NEB (MA, USA) | R0569L |
| Q5 ® High-Fidelity DNA Polymerase | NEB (MA, USA) | M0491S |
| Q5 ® High-Fidelity PCR Kit | NEB (MA, USA) | E0555S |
| Deoxynucleotide (dNTP) Solution Mix | NEB (MA, USA) | N0447S |
| Quick-Load ® Purple 100 bp DNA Ladder | NEB (MA, USA) | N0551S |
| Gel Loading Dye, Purple | NEB (MA, USA) | B7024S |
| T4 DNA Ligase | NEB (MA, USA) | M0202L |
| TrackIt ™ 100 bp DNA Ladder | Thermo Scientific ™ (MA, USA) | 10488058 |
| UltraPure ™ Agarose | Thermo Scientific ™ (MA, USA) | 16500100 |
| 2x YT media | Sigma-Aldrich (MA, USA) | Y2377-250G |
| Polyethylene glycol | Sigma-Aldrich (MA, USA) | 81260-1KG |
| Platinum ™ II Hot-Start Green PCR Master | Thermo Scientific ™ (MA, USA) | 14001012 |
| Mix | | |
| TG1 Electrocompetent Cells | Lucigen (WI, USA) | 60502-2 |
| Recombinant human ErbB 2 protein | Abcam (MA, USA) | ab168896 |
| Recombinant Human ErbB 3 protein | Abcam (MA, USA) | ab196072 |
| Recombinant human ErbB 4 protein | Abcam (MA, USA) | ab85602 |
| Recombinant human EGFR protein (Fc | Abcam (MA, USA) | ab155726 |
| Chimera) | | |
| Goat Anti-Rabbit IgG H&L (FITC) | Abcam (MA, USA) | ab6717 |
| Goat Anti-Human IgG H&L (HRP) | Abcam (MA, USA) | ab97165 |
| Goat Anti-Human IgG H&L (FITC) | Abcam (MA, USA) | ab6854 |
| Human IgG1, kappa | Abcam (MA, USA) | ab206198 |
| Anti-c-Myc antibody (HRP) | Abcam (MA, USA) | ab19312 |
| Anti-HA tag antibody (HRP) | Abcam (MA, USA) | ab1190 |
| Llama IgG-heavy and light chain Antibody | Bethyl (TX, USA) | A160-100P |
| Histag Polyclonal Antibody, DyLight 680 | Thermo Scientific ™ (MA, USA) | 600-444-382 |
| Biotinylated Human Her2/ErbB2 Protein, | AcroBiosystem (DE, USA) | HE2-H82E2-25ug |
| His, Avitag ™ | | |
| Trastuzumab (Trazimera ™, Pfizer) | Cancer Institute of New Jersey | N/A |
| | Pharmacy Store | |
| Pertuzumab (Perjeta ™, Genentech) | Cancer Institute of New Jersey | N/A |
| | Pharmacy Store | |
| BT-474 breast cancer cells | ATCC (VA, USA) | HTB-20 ™ |
| SK-BR-3 breast cancer cells | ATCC (VA, USA) | HTB-30 ™ |
| SKOV-3 ovarian cancer cells | ATCC (VA, USA) | HTB-77 ™ |
| MDA-MB-231 | ATCC (VA, USA) | HTB-26 ™ |
| Hybri-Care Medium | ATCC (VA, USA) | 46-X |
| Leibovitz's L-15 Medium | ATCC (VA, USA) | 30-2008 |
| DMEM/F-12, GlutaMAX ™ supplement | Gibco ™ (NY, USA) | 10565018 |
| Fetal Bovine Serum, qualified, United States | Gibco ™ (NY, USA) | 26140079 |
| Penicillin-Streptomycin (10,000 U/mL) | Gibco ™ (NY, USA) | 15140122 |
| Insulin (10 mg/mL) | Sigma-Aldrich (MA, USA) | I0516-5ML |
| McCoy's 5A (Modified) Medium | Gibco ™ (NY, USA) | 16600108 |
| RPMI-1640 Medium | Sigma-Aldrich (MA, USA) | R8758-6X500ML |

### Expression and Purification of HER2 Protein

The gene encoding the extracellular domain of HER2 (Uniprot ID P04626) was designed, synthesized, and cloned into a piggyback plasmid vector (eHER2bac) by VectorBuilder (IL, USA) downstream of an EF1α promoter. A secretory signal was designed at N-terminal and 12XHistag (SEQ ID NO: 2) at C-terminal of the protein sequence to facilitate secretion of the expressed protein into the culture media and purification by Ni-NTA chromatography, respectively (Table 5). The FreeStyle™ 293-F Cell system was chosen to carry out protein expression since human HER2 protein is heavily glycosylated. On the day of transfection, 293-F cells were counted and resuspended at the density of ˜3×10⁶ cells/ml in 100 mL in a 250 mL Reusable Spinner Flask containing fresh FreeStyle™ 293 expression media and incubated at 37° C. (5% CO2) for 30 min. To transfect the 293-F cells, plasmid DNA (eHER2bac) was complexed with PEI (1 μg per 10⁶ cells) at 1:4 ratio (pDNA:PEI) and incubated at room temperature (RT) for 20 min. Then, pDNA:PEI complexes were added to 293-F cells dropwise under constant stirring. The flask containing the transfected 293-F cells was transferred into a CO₂ incubator and stirred at 90 rpm for 24 h. The next day, two-fold fresh media (200 mL) was added to the transfected cells and the protein expression continued for 8 to 10 days until the cell viability dropped below 85% (determined by Trypan Blue). Then, cells were collected by centrifugation (10,000 g, 10 min, 4° C.) and the supernatant containing protein of interest was transferred into a fresh tube and incubated with 300 μL of Ni-NTA resin overnight at 4° C. while shaking. The next day, the resins were loaded onto a chromatography column and washed with 20 mL of wash buffer (500 mM NaCl, 20 mM Na₂HPO₄, 50 mM Tris, 7.5 mM imidazole, pH 7.4). The purified HER2 protein was eluted using 500 μL of elution buffer (500 mM NaCl, 20 mM Na₂HPO₄, 50 mM Tris, 250 mM imidazole, pH 7.4). The purity of the eluted HER2 protein was evaluated by SDS-PAGE.

TABLE 5
The amino acid sequence of the secretory recombinant HER2 protein cloned into
eHER2bac plasmid with theoretical molecular weight of 73.56 kDa (SEQ ID NO: 21)

| Name | Amino Acid Sequence |
|---|---|
| HER2 | METDTLLLWVLLLWVPGSTGDAAQPARRASLWELQTQVCTGTDMKLRLPASPET |
| Extracellular | HLDMLRHLYQGCQVVQGNLELTYLPTNASLSFLQDIQEVQGYVLIAHNQVRQVPL |
| Domain | QRLRIVRGTQLFEDNYALAVLDNGDPLNNTTPVTGASPGGLRELQLRSLTEILKGGV |
| | LIQRNPQLCYQDTILWKDIFHKNNQLALTLIDTNRSRACHPCSPMCKGSRCWGESSE |
| | DCQSLTRTVCAGGCARCKGPLPTDCCHEQCAAGCTGPKHSDCLACLHFNHSGICEL |
| | HCPALVTYNTDTFESMPNPEGRYTFGASCVTACPYNYLSTDVGSCTLVCPLHNQEV |
| | TAEDGTQRCEKCSKPCARVCYGLGMEHLREVRAVTSANIQEFAGCKKIFGSLAFLP |
| | ESFDGDPASNTAPLQPEQLQVFETLEEITGYLYISAWPDSLPDLSVFQNLQVIRGRIL |
| | HNGAYSLTLQGLGISWLGLRSLRELGSGLALIHHNTHLCFVHTVPWDQLFRNPHQA |
| | LLHTANRPEDECVGEGLACHQLCARGHCWGPGPTQCVNCSQFLRGQECVEECRVL |
| | QGLPREYVNARHCLPCHPECQPQNGSVTCFGPEADQCVACAHYKDPPFCVARCPSG |
| | VKPDLSYMPIWKFPDEEGACQPCPINCTHSCVDLDDKGCPAEQRASPLTWELQLEE |
| | GPHHHHHHHHHHHH* |
| | (SEQ ID NO: 21) |

*Secretory signal (BOLD) - Protease site (italics) - HER2 ectodomain - Histag (HHHHHHHHHHHH (SEQ ID NO: 2))

Immunization of llama with HER2 Protein

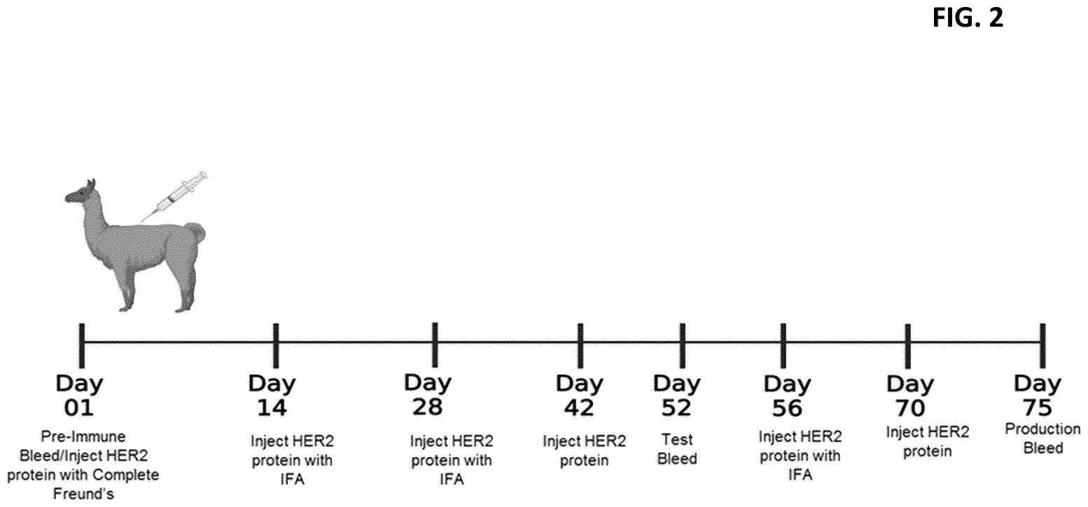

The purified HER2 protein was sent to Capralogics Inc. (Gilbertville, MA) to immunize llama (FIG. 2). A 1.5-year-old female llama was immunized six times (every two weeks) using 500 μg of HER2 protein per injection. In four out of six injections, HER2 protein was mixed with either complete or incomplete Freund's adjuvant to maximize the immune response. In the fourth and sixth injection, the purified HER2 was injected without any adjuvant. Ten days after fourth injection, on day 52, 50 ml serum was collected, and an ELISA was performed to measure the IgG levels in Llama serum. Once the IgG response was confirmed, the immunization procedure was continued for four more weeks. Five days after the last injection, 600 ml of Llama blood was collected and then diluted with 600 ml of DPBS supplemented with 3% FBS.

### Isolation of Peripheral Blood Mononuclear Cells (PBMCs)

Total PBMCs were isolated using Ficoll (Histopaque®-1077) method. In brief, 10 mL Ficoll was added to a 50 ml tube. Then, 20 ml of diluted blood was added dropwise in such a way that the interface between blood and Ficoll remained undisturbed. Next, red blood cells and granulocytes were separated from PBMCs by centrifugation (400 g, 20 min, RT) with the brake off. This process enriched PMBCs in a layer between serum and Ficoll. The serum was slowly removed and PBMCs were collected into a new tube and washed twice using DPBS supplemented with 3% FBS. The isolated PBMCs were used for library generation.

### Library Generation

Using isolated PBMCs, the total RNA was extracted (RNeasy Mini Kit), and cDNA was synthesized using oligo-dT and Random Hexamer (SuperScript™ IV First-Strand Synthesis System). The VHH cDNAs were amplified by nested PCR (Q5® High-Fidelity DNA Polymerase) using two sets of primers (Table 6) and amplification protocol (Table 7). The PCR products were cloned into pMECS-GG phagemid (Kindly provided by Dr. S. Muyldermans, Belgium) using SapI restriction enzyme and T4 DNA ligase using golden gate cloning protocol. Recombinant phagemids were then transformed into TG1 bacteria by an electroporator using 0.1 cm gap electroporation cuvettes. Briefly, 5 μL of recombinant phagemid harboring VHH sequence mixed with 50 μL competent TG1 cells and transferred into electroporation cuvette. After 20 min on ice, electroporation was performed using voltage setting of 2.5 kV, resistance at 200Ω and capacitance at 25 μF. Ten vials of competent TG1 were transformed to keep the diversity of the library. Then 1 ml of SOC medium was added and transferred into a 50 ml tube. After 60 min incubation at 37° C., the bacteria were harvested at 6,000×g for 15 min. The pellet was transferred into six flasks of 100 mL LB supplemented with 100 μg/mL Carbenicillin. The next day, the bacteria from all six flasks were spun down and resuspended in 20 mL of fresh LB media. After adding 15% glycerol, the library was stored at −80° C. for phage display.

TABLE 6
The list of primers with corresponding sequences that were used
to make VHH cDNAs.

| Name | Sequence |
|---|---|
| CALL001 | 5′- GTCCTGGCTGCTCTTCTACAAGG-3′ (SEQ ID NO: 22) |
| CALL002 | 5′- GGTACGTGCTGTTGAACTGTTCC-3′ (SEQ ID NO: 23) |
| VHH-Back | 5′- GATGGGCTCTTCTGTGTGCAGCTGCAGGAGTCTGGRGGAGG-3′ |
| | (SEQ ID NO: 24) |
| VHH-Forward | 5′-CTAGTGCTCTTCCGCTTGGAGACGGTGACCTGGGT-3′ |
| | (SEQ ID NO: 25) |

TABLE 7
The PCR protocol for the amplification of the primers.

| Cycle | Step | Temperature | time |
|---|---|---|---|
| 1 X | Initial Denaturation | 95 C. | 3 min |
| 35 X | Denaturation | 95 C. | 1 min |
| | Annealing | 50-60 C. | 1 min |
| | Synthesis | 72 C. | 1 min |
| 1 X | Final Extension | 72 C. | 10 min |
| 1 X | | 4 C. | — |

### Phage Display

Four rounds of panning were carried out to reach a specific VHH library. Each round was carried out as follows. First, around 2 OD₆₀₀ₙₘ (1 OD_{600 nm}=2.66×10⁹ cells/mL) of VHH library was inoculated into 100 mL LB broth. When the OD₆₀₀ reached 0.6-0.8, 500 μL of VCSM13 helper phage was added and incubated at 37° C. for 1-2 h without shaking. Then 50 μg/ml Kanamycin was added, and bacterial culture continued overnight. For phage display, 100 μL of HER2 (1 μg/mL) protein was used to coat a 96-well plate and incubated at 4° C. overnight. The following day, bacteria was pelleted by centrifugation (10,000 g, 20 min, 4° C.). The supernatant was transferred into a sterile, pre-chilled 50 mL tube and 10% PEG-NaCl was added and incubated at 4° C. for 1-2 h. Afterward, phages were collected by centrifugation (3,000 g, 20 min, at 4° C.). The collected phages were washed twice using DPBS. Next, while the ELISA plate was being blocked using 2% skimmed milk buffer, recombinant phages were incubated with blocking buffer while shaking. Afterward, 100 μL of recombinant phage displaying VHH of interest was added to a HER2-coated plate and incubated for 1 h at room temperature (RT) while shaking at 700 rpm. After washing ten times using DPBS/0.1% Tween and ten times using DPBS, bound phages were collected enzymatically using trypsin (0.25%). Next, 10 μL of harvested phages were incubated with 100 μL TG1 bacteria, at exponential growth, for 1 h at 37° C. Then, 100 μL of infected TG1 was transferred into 100 mL LB media containing 100 μg/mL Carbenicillin, and bacterial culture continued overnight.

### Preparation of Periplasmic Extract and Evaluation of Affinity and Specificity by ELISA

After four rounds of phage display, 190 colonies were screened to find the clone with the highest affinity toward HER2. First, 1 mL of LB broth supplemented with 100 μg/mL carbenicillin was added to a Deep-Well 96-well plate. The plate was covered by a ventilating adhesive plate seal to minimize potential contamination and evaporation. Next, one colony was inoculated into each well and incubated at 37° C. overnight. The next day, 10 μL of overnight culture was transferred into a new Deep-Well 96-well plate containing 1 mL of LB broth supplemented with 100 μg/mL carbenicillin and incubated at 37° C. for 4 h. Afterwards, the protein expression was induced by adding 1 mM IPTG and the bacterial culture was grown overnight at 28° C. The next day, the plate was spun down at 10,000×g for 10 min at 4° C., and the supernatants were discarded. To prepare the periplasmic extract, pelleted bacteria underwent freeze-thaw cycle three times (30 min at −20° C. followed by 10 min at RT). Then, 500 μL DPBS was added to each pellet and incubated at RT for 30 min while shaking. The cell debris were removed by centrifugation and 400 μL of supernatant was gently transferred into a new 96-well plate and stored at 4° C.

For determination of affinity and specificity by ELISA, 100 μL of 1 μg/mL HER2 protein and three other members of Heregulin superfamily including HER1, HER3, and HER4 were used to coat Nunc MaxiSorp™ high protein-binding capacity 96-well ELISA plates and incubated overnight at 4° C. The next day, each well was washed three times with washing buffer (DPBS+0.1% Tween 20) and then incubated with blocking buffer (2% skimmed milk) for 2 h at RT to block the free binding sites. Next, blocking buffer was replaced with 100 μL of periplasmic extract from above and incubated for 1 h at RT while shaking at 700 rpm. Each well was washed six times with washing buffer (DPBS+0.1% Tween 20) followed by addition of 100 μL HRP Anti-HA tag antibody (1:10000 dilution) and incubation for 1 h at RT while shaking at 700 rpm. Each well was washed again six times, followed by addition of 50 μL 1-Step™ Turbo TMB-ELISA substrate solution at incubation at RT for 15 min (without shaking and at dark). The reaction was stopped by adding 50 μL of stop solution (0.1 mM sulfuric acid) and the plate was read at 450 nm using 630 nm as a reference (450/630 nm).

### Expression and Purification of Anti-HER2 VHHs

The DNA sequences encoding the selected VHHs were codon optimized, synthesized, and cloned into pHEN6c plasmid (Kindly provided by Dr. S. Muyldermans, Belgium) by GeneWiz (NJ, USA). The recombinant pHEN6c plasmids harboring VHH sequences were transformed into WK6 E. coli using heat shock. A 750 mL TB media was supplemented with 0.1% Glucose, 1 mM MgCl₂ and 100 μg/mL carbenicillin. The media was then inoculated with 10 mL of WK6 bacterial culture and grown at 37° C. When the OD₆₀₀ reached 0.5-0.8, the protein expression was induced by 0.5 mM IPTG. Protein expression continued at 28° C. overnight while shaking at 180 rpm. The next day, the bacterial culture was centrifuged (10,000×g, 10 min, 4° C.) to pellet the bacteria. The pellet was resuspended in 10 mL of TES buffer (200 mM Tris-HCl, pH 8.0, 500 mM sucrose, 1 mM EDTA) and incubated on ice for 1 h while shaking. Then, 15 mL of TES buffer was diluted with distilled water to a total volume of 60 mL, added to the cell suspension, and incubated on ice for additional 45 min while shaking. The bacteria were pelleted by centrifugation at 15,000×g, 60 min, 4° C. Then, the supernatant was loaded onto a Ni²⁺ column and washed with 30 mL of wash buffer (500 mM NaCl, 20 mM Na₂HPO₄, 50 mM Tris, 25 mM imidazole, pH 7.4). Finally, the protein was eluted by elution buffer (500 mM NaCl, 20 mM Na₂HPO₄, 50 mM Tris, 250 mM imidazole, pH 7.4). The purity of the eluted protein (VHH) was evaluated by SDS-PAGE.

### Evaluation of the Ability of VHHs to Recognize HER2+ Cells by Using Flow Cytometry

BT474 (Hybri-Care Medium/10% FBS), SKOV-3 (McCoy's 5A/10% FBS), MDA-MB-231 (Leibovitz's L-15 Medium/10% FBS), and OVASC-1 (RPMI-1640/15% FBS/2.5 ug/mL insulin) cancer cells were cultured in the corresponding culture media. OVASC-1 is an ascites-derived epithelial ovarian cancer cells obtained from a patient with advanced metastatic disease (11, 12). The expressed VHHs were then incubated with HER2⁺ (BT474, JIMT-1, and SKOV-3) and HER2⁻ (MDA-MB-231) cancer cell lines. Approximately 10⁶ cells were counted and washed using DPBS-1% FBS. Then, equivalent to 100 nM binding sites of Trastuzumab, Perjeta, or VHHs were added to the cells and incubated on ice for 60 min. After washing, FITC-conjugated anti-Human IgG or anti-Histag antibody were added and incubated on ice for 60 min. Cells were washed three times and then analyzed using Beckman-Coulter Gallios flow cytometer at Rutgers Flow cytometry core facility.

### Measurement of VHH Binding Affinity and Kinetics Using Biolayer Interferometer

To evaluate the binding affinity (K_{D}) and constant rates of association (Kₒₙ) and dissociation (K_{off}) of VHHs, an Octet RED96e instrument (Sartorius) Biolayer Interferometer (BLI) along with an Octet® Streptavidin (SA) Biosensor were used. In this experiment, Trastuzumab and Pertuzumab were used as positive controls. After soaking the sensor for at least 10 min in DPBS, biotinylated HER2 was loaded onto the Streptavidin (SA) Biosensor for 10 min to coat the streptavidin on sensor with biotinylated HER2. Next, the sensor was dipped into the washing buffer (DPBS+0.05% Tween 20) for 2 min to reach the baseline. Then, sensor was submerged into wells containing 100, 50, 25, 12.5, 6.25, 3.25 and 0 nM of purified anti-HER2 VHH, Herceptin®, or Perjeta® for 5 min (association step). In dissociation step, wells were dipped into washing buffer (DPBS+0.05% Tween 20) for 10 min to acquire data (Table 8). The data were then analyzed using Octet Data Analysis HT 11.1 software. For data analysis, the sensograms were subtracted from the reference, and fitted into 1:1 and 2:1 binding models. Finally, the affinity and kinetics were analyzed using “Association and Dissociation”. All BLI data acquisition and analysis studies were performed at Biophysics Core Facility in the Department of Chemistry at Princeton University.

TABLE 8
The experimental conditions used to acquire data from the BLI.

| Step | Name | Time (s) | Shake Speed |
|---|---|---|---|
| 1 | Sensor Check | 30 | 1000 |
| 2 | Loading (10 μg/mL HER2) | 600 | 1000 |
| 3 | Baseline (DPBS + 0.05% Tween 20) | 120 | 1000 |
| 4 | Association | 300 | 1000 |
| 5 | Dissociation (DPBS + 0.05% Tween 20) | 600 | 1000 |

### Cell Cytotoxicity Assay

The toxicity of anti-HER2 VHHs, Herceptin®, and Perjeta®, were evaluated using BT-474 and MDA-MB-231 cancer cells. Briefly, 10×10⁶ cells were seeded in a 96-well plate. The next day, cells were treated with 1333.3, 13.3, 13, 0.13, 0.013 and 0 nM of VHH or antibody. After 72 h, the media was removed, and 100 μL of fresh media supplemented with 10% WST-1 reagent was added. After 4 h, the plate was read at 440 nm and 600 nm as a reference wavelength. All experiments were performed in triplicates and data are presented as mean±s.d.

### Evaluation of the Application of Anti-HER2 VHH in Cell Imaging

The internalization of VHH after binding to HER2 receptor in SK-BR-3 cancer cells was examined using confocal microscopy. SK-BR-3 cells were seeded at the density of 2.5×10⁴ cells on a Nunc Lab-Tek™ chamber slide. The next day, cells were treated with 20 nM of VHH or Herceptin® (trastuzumab) and incubated on ice for 60 min. Next, the antibody solution was discarded, and all wells were washed three times using ice-cold DPBS. The cells were fixed by 3.7% paraformaldehyde for 15 min at 37° C. followed by three steps of washing. To examine the rate of internalization at different time points, RPMI media supplemented with 5% FBS was added to the to the wells and incubated at 37° C. for 1, 2, and 4 hrs followed by washing and fixation. Afterwards, cells were permeabilized using 0.1% Triton X100 for 15 min at RT. Cells were incubated with blocking buffer (DPBS with 2% BSA) for 2 h at RT. Next, secondary antibody was added and incubated at RT for 1 h. After three steps of washing, cells were stained with 300 nM DAPI for 3 min followed by three times wash. Finally, HER2 internalization was observed under a Leica TCS SP8 Confocal Microscope (Leica Microsystems GmbH) with the 63× objective lens using immersion oil. Z-stack images were taken each at 500 nm apart. The images of different z-stacks were processed by Leica software.

### Results and Discussion

Expression of HER2 and Immunization of llama

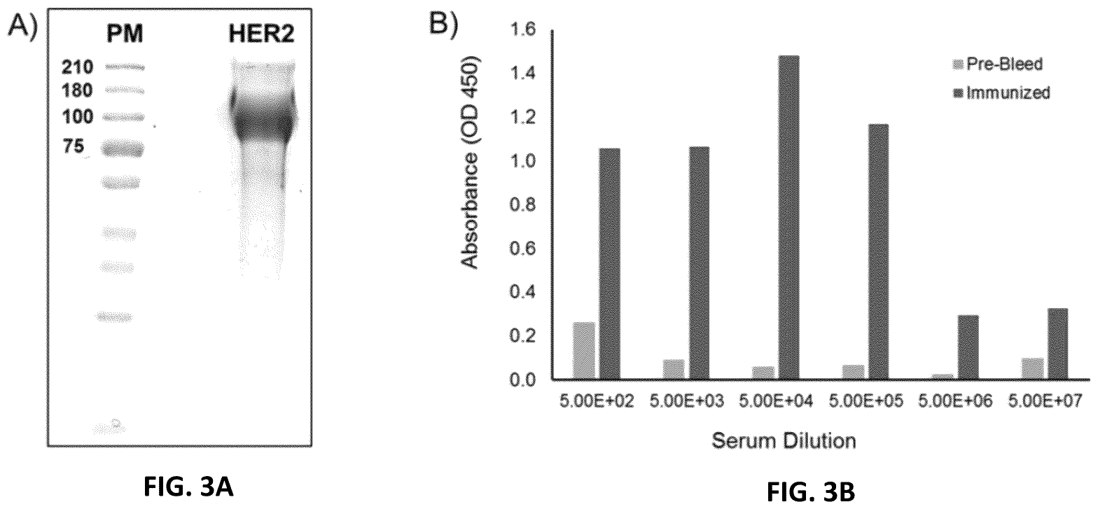

Using standard genetic engineering techniques, the HER2 antigen was expressed in HEK293 cells and purified. The SDS-PAGE results estimated the purity of the purified HER2 to be approximately 95% (FIG. 3A). While the theoretical molecular weight of the HER2 (without secretory signal) is 72,633 Da, the migration of the protein is close to the 100 kDa marker. This indicates that the expressed HER2 was glycosylated. The purified HER2 was then used to immunize llama. Blood draw four weeks post immunization followed by ELISA showed significant elevation in IgG levels (OD₄₅₀ of 1.5 after 1/20,000 dilution) indicating potent immune response to the injected HER2 antigen (FIG. 3B).

### Isolation of PBMCs, Library Generation, Phage Display, and Colony Screening

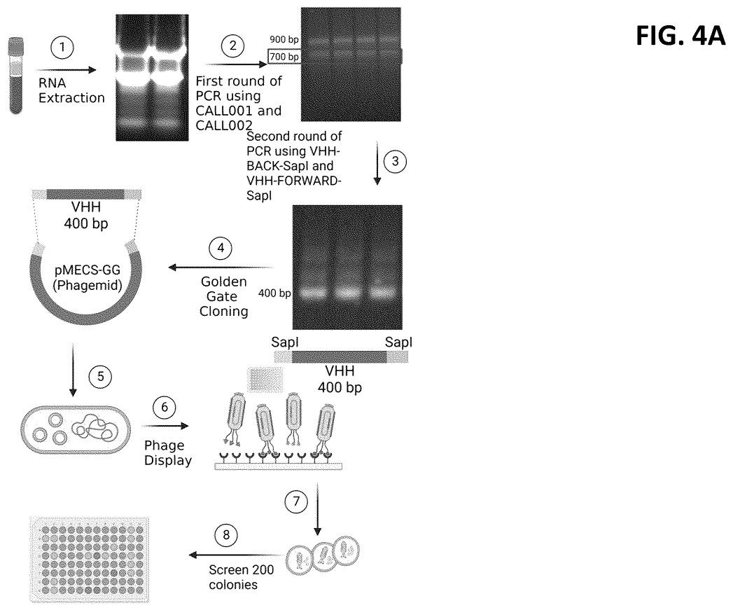

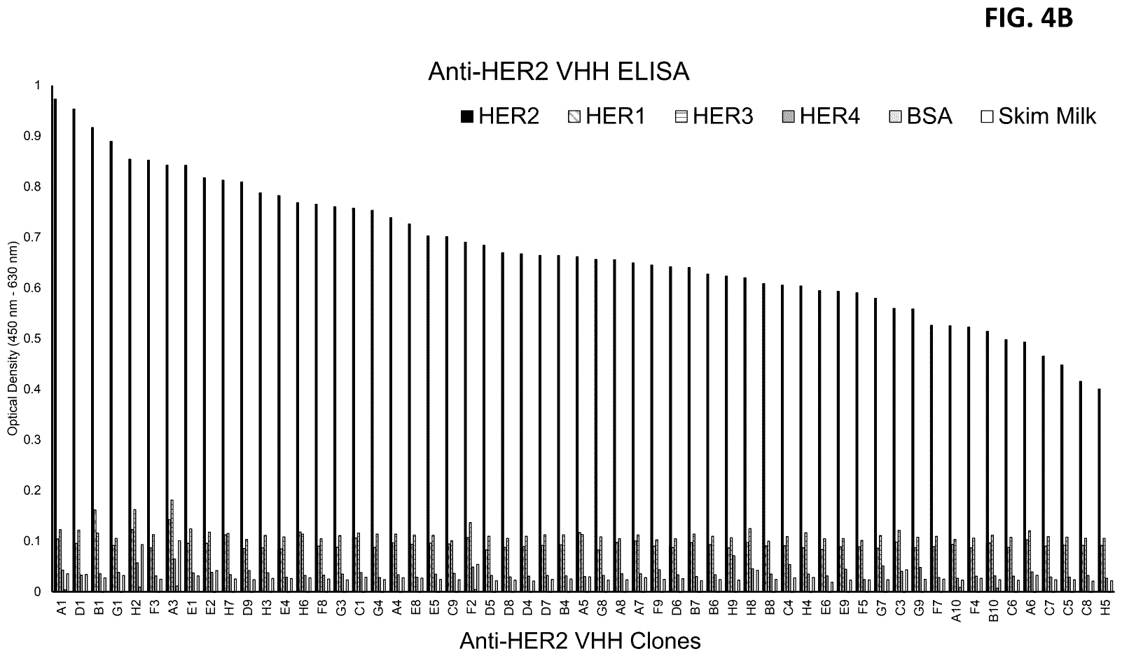

On day 75 post immunization of llama, blood was drawn, PBMCs isolated, a cDNA and phagemid library were generated, and then used in phage display. After four rounds of phage display, colonies with highest binding affinity to HER2 antigen were selected and then examined for specificity and selectivity (FIG. 4A). The results of these experiments showed that the isolated VHHs were specific and selective to HER2 antigen and with negligible interaction with HER1, HER3, HER4, BSA, and skim milk (FIG. 4B).

### VHH Selection, Expression, and Purification

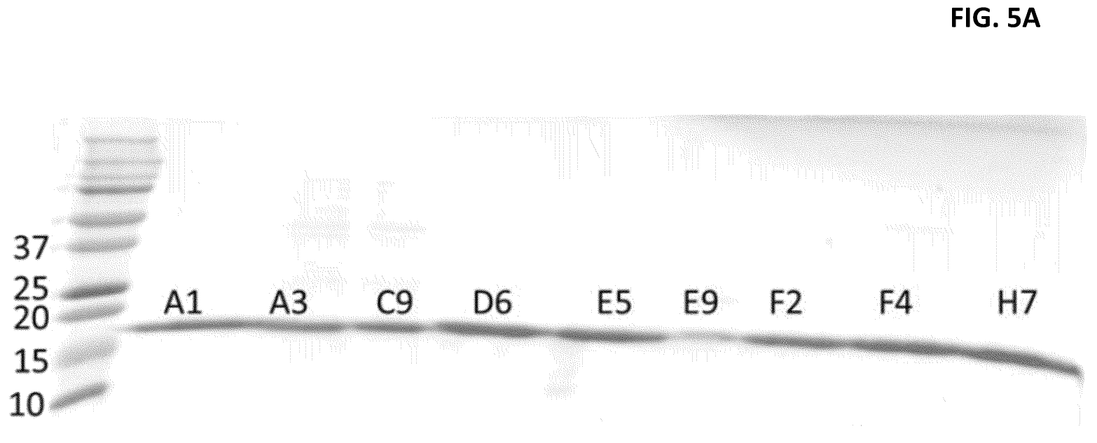

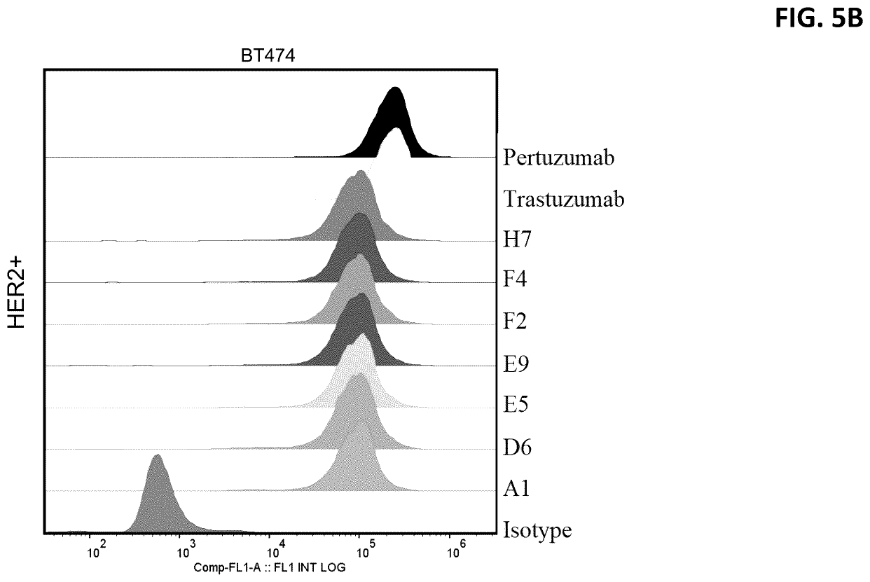

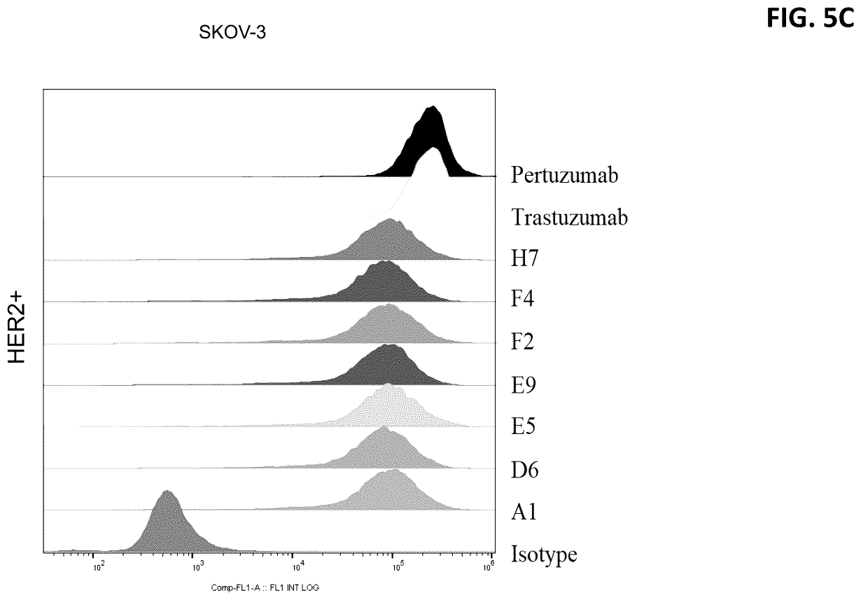

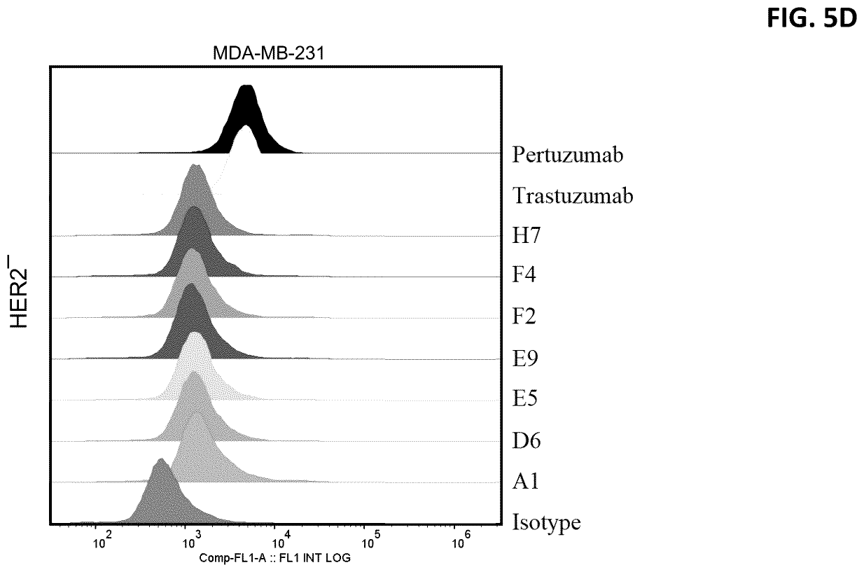

All forty HER2-binding VHHs were sequenced, analyzed, and grouped based on the phylogenic tree. One sequence of each group with the highest affinity and specificity to HER2 was selected and then constructed to have a c-myc and histag at its C-terminal (Table 9). Addition of these two tags allow for the VHHs to be recognized by anti-c-myc and anti-histag antibodies. The top-performing VHHs were then expressed in WK6 E. coli and purified. The yield of expression was between 3-5 mg/l for all VHHs and with the estimated purity of above 95% (FIG. 5A).

TABLE 9
The amino acid sequences of the selected VHHs. The c-myc (GSEQKLISEEDL (SEQ
ID NO: 1)) and histidine (HHHHHHHHHHHH (SEQ ID NO: 2)) tags are constructed at VHH
C-terminal, respectively.

| Anti- | | | | | | | | |
|---|---|---|---|---|---|---|---|---|
| HER2 | | | | | | | | |
| VHH | | | | | | | Framework | |
| Codes | Framework 1 | CDR1 | Framework 2 | CDR2 | Framework 3 | CDR3 | 4 | Tag |
| A1 | QVQLQESGG | GHTLSTYV | VAWFRQAPG | IGWSSRSP | YYPDSVKGRFTISEDN | AADSYPRRW | WGQGTQVTV | C-Myc + |
| (SEQ ID | GLVQPGGSL | (SEQ ID | KERESVAA | (SEQ ID NO: | TKNTVYLQMNSLKPD | DRESDFGS | SS (SEQ | Histag |
| \|NO 11) | RLSCAAS | NO: 41) | (SEQ ID NO: | 43) | DTAVYYC (SEQ ID | (SEQ ID | ID NO: 34) | |
| | (SEQ ID NO: | | 42) | | NO: 44) | NO: 45) | | |
| | 40) | | | | | | | |
| A3 | QVQLQESGG | GRTSDLFP | VGWFRQAP | IRWSGGVI | GYGDPVKGRFTISTD | AAGPRES | WGQGTHVTVS | C-Myc + |
| (SEQ ID | GLVQAGGSL | (SEQ ID | GKEREIVAA | (SEQ ID NO: | NAKNTVYLQMNSLK | TVTWNY | S (SEQ | Histag |
| NO 3) | RISCAAS | NO: 47) | (SEQ ID NO: | 49) | PEDTAVYYC (SEQ ID | (SEQ ID | ID NO: 52) | |
| | (SEQ ID NO: | | 48) | | NO: 50) | NO: 51) | | |
| | 46) | | | | | | | |
| C9 | QVQLQESGG | GRTVSRYS | MGWFRQAP | IAWFGGWP | YYADSVKGRFTISRDN | AARQWAR | WGQGTQVTV | C-Myc + |
| (SEQ ID | GLVQAGGSL | (SEQ ID | GKEREFVAA | (SEQ ID NO: | AKNTVYLQMNSLKPE | TWDY | SS (SEQ | Histag |
| NO 4) | RLSCAAS | NO: 53) | (SEQ ID NO: | 55) | DTAVYYC (SEQ ID | (SEQ ID | ID NO: 34) | |
| | (SEQ ID NO: | | 54) | | NO: 56) | NO: 57) | | |
| | 28) | | | | | | | |
| D6 | QVQLQESGG | GRTFSTYV | VGWFRQAP | IGWASNSP | YYADSVKGRFTISRDN | AADSYPRRW | WGQGTQVTV | C-Myc + |
| (SEQ ID | GLVQAGGSL | (SEQ ID | GKERESVAA | (SEQ ID NO: | TKNTVYLQMKSLKPD | DRESDFGS | SS (SEQ | Histag |
| NO 5) | TLSCAAS | NO: 59) | (SEQ ID NO: | 61) | DTAVYYC (SEQ ID | (SEQ ID | ID NO: 34) | |
| | (SEQ ID NO: | | 60) | | NO: 62) | NO: 45) | | |
| | 58) | | | | | | | |
| E5 | QVQLQESGG | GRTLSSYV | VGWFRQAP | IGWSRTST | FYTDSVKGRFTISRDN | AADSSPRRW | WGQGTQVTV | C-Myc + |
| (SEQ ID | GLVQAGGSL | (SEQ ID | GKEREVVAA | (SEQ ID NO: | TENTVYLQMNSLKPG | DRESDFGS | SS (SEQ | Histag |
| NO 6) | RLDCAAS | NO: 64) | (SEQ ID NO: | 66) | DTAVYYC (SEQ ID | (SEQ ID | ID NO: 34) | |
| | (SEQ ID NO: | | 65) | | NO: 67) | NO: 68) | | |
| | 63) | | | | | | | |
| E9 | QVQLQESGG | GRTLSAYV | VGWFRQAP | IGWSTHSV | YYADSVKGRFTISRDN | AADSSPRRW | WGQGTQVTV | C-Myc + |
| (SEQ ID | GSVQAGGSL | (SEQ ID | GRERESVAA | (SEQ ID NO: | TENTVYLQMNSLKPD | DRESDFGS | SS (SEQ | Histag |
| NO 7) | KLSCAAS | NO: 70) | (SEQ ID NO: | 72) | DTAVYYC (SEQ ID | (SEQ ID | ID NO: 34) | |
| | (SEQ ID NO: | | 71) | | NO: 73) | NO: 68) | | |
| | 69) | | | | | | | |
| F2 | QVQLQESGG | GRTLSAYV | VGWFRQAP | IGWSTHSV | YYADSVKGRFTISRDN | AADSSPRRW | WGQGTQVTV | C-Myc + |
| (SEQ ID | GLVQAGGSL | (SEQ ID | GRERESVAA | (SEQ ID NO: | TENTVYLQMNSLKPD | DRESDFGS | SS (SEQ | Histag |
| NO 8) | KLSCAAS | NO: 70) | (SEQ ID NO: | 72) | DTAVYYC (SEQ ID | (SEQ ID | ID NO: 34) | |
| | (SEQ ID NO: | | 71) | | NO: 73) | NO: 68) | | |
| | 74) | | | | | | | |
| F4 | QVQLQESGG | GRTLSAYV | VGWFRQAP | IGWSSRSP | YYPDSVKGRFTISEDN | AADSYPRRW | WGQGTQVTV | C-Myc + |
| (SEQ ID | GLVQAGGSL | (SEQ ID | GKERESVAA | (SEQ ID NO: | TKNTVYLQMNSLKPD | DRESDFGS | SS (SEQ | Histag |
| NO 9) | KLSCAAS | NO: 70) | (SEQ ID NO: | 43) | DTAVYYC (SEQ ID | (SEQ ID | ID NO: 34) | |
| | (SEQ ID NO: | | 60) | | NO: 44) | NO: 45) | | |
| | 74) | | | | | | | |
| H7 | QVQLQESGG | GHTLSTYV | VAWFRQAPG | IGWSTRSP | YYADSVKGRFTISEDN | AADSYPRRW | WGQGTQVTV | C-Myc + |
| (SEQ ID | GLVQAGGSL | (SEQ ID | KERESVAA | (SEQ ID NO: | TKNTVYLQMNSLKPD | DRESDFGS | SS (SEQ | Histag |
| NO 10) | RLSCAAS | NO: 41) | (SEQ ID NO: | 75) | DTAVYYC (SEQ ID | (SEQ ID | ID NO: 34) | |
| | (SEQ ID NO: | | 42) | | NO: 76) | NO: 45) | | |
| | 28) | | | | | | | |

### Evaluation of the Application of the Anti-HER2 VHHs in Flow Cytometry

In the next step, the ability of the VHHs to recognize and bind to HER2 on the surface of HER2+ cancer cells was evaluated. For this purpose, the equimolar binding sites of purified VHHs and FDA-approved anti-HER2 antibodies (Trastuzumab and Pertuzumab) were used to measure the HER2 expression on the surface of BT474 and SKOV-3 (HER2+) HER2+ cancer cells. MDA-MB-231 and OVASC-1 (HER2⁻) cancer cells were used as negative controls. The flow cytometry data showed that the selected VHHs can recognize HER2 on the surface of HER2+ cancer cells but not on HER2⁻ cancer cells (FIGS. 5B-5E). The total mean fluorescent 10 intensity (MFI) of cells that were labeled with Trastuzumab and Pertuzumab appeared to be higher than VHHs. The major reason is that the secondary antibody that was used to detect Trastuzumab and Pertuzumab was polyclonal against light and heavy chain (1 antibody/multiple labels) resulting in a significant signal boost (Table 10). In contrast, the secondary antibody that was used to detect histag in VHHs was monoclonal (1 VHH/1 label) which generated lower signal intensity. Nonetheless, the level of HER2 binding by VHHs, as determined by the percentage of labeled cells, was similar to Trastuzumab and Pertuzumab (Table 10).

Overall, the flow cytometry data show that the selected VHHs can differentiate HER2+ from the HER2⁻ cells, and can be used as a suitable reagent for cell phenotyping by flow cytometry.

TABLE 10
The MFI and percentages of the HER2+ and HER2⁻ cancer cells as labeled by VHHs, Trastuzumab, and Pertuzumab.

| | HER2+ | | | | | | HER2⁻ | | | | | |
|---|---|---|---|---|---|---|---|---|---|---|---|---|
| | BT-474 | | | SKOV-3 | | | OVASC-1 | | | MDA-MB-231 | | |
| | FITC+ | FITC⁻ | | FITC+ | FITC⁻ | | FITC+ | FITC⁻ | | FITC+ | FITC⁻ | |
| Name | (%) | (%) | Mean | (%) | (%) | Mean | (%) | (%) | Mean | (%) | (%) | Mean |
| Pertuzumab | 98.8 | 1.24 | 233983 | 98.4 | 1.49 | 354125 | 10.3 | 89.7 | 6180 | 68.9 | 31.1 | 3580 |
| Trastuzumab | 98.5 | 1.54 | 251072 | 97.6 | 1.86 | 425871 | 12 | 88 | 6283 | 67.5 | 32.5 | 5858 |
| H7 | 98.3 | 1.72 | 100269 | 94.8 | 5.02 | 99877 | 6.62 | 93.4 | 4249 | 4.92 | 95.1 | 2081 |
| F4 | 98.5 | 1.45 | 99514 | 93.3 | 6.51 | 86366 | 0.15 | 99.8 | 1294 | 5.06 | 94.9 | 2005 |
| F2 | 98.6 | 1.42 | 99461 | 93.1 | 6.68 | 100019 | 7.83 | 92.2 | 4709 | 5.43 | 94.6 | 1937 |
| E9 | 98.2 | 1.85 | 97925 | 93.1 | 6.76 | 88102 | 0.13 | 99.9 | 1327 | 5.31 | 94.7 | 1893 |
| E5 | 98.4 | 1.59 | 102038 | 93.3 | 6.45 | 106010 | 4.89 | 95.1 | 3689 | 5.79 | 94.2 | 1989 |
| D6 | 98.1 | 1.86 | 96119 | 94.6 | 5.24 | 89442 | 6.52 | 93.5 | 4327 | 5.94 | 94.1 | 2113 |
| A1 | 98.8 | 1.23 | 97001 | 94.1 | 5.78 | 100706 | 6.46 | 93.5 | 4703 | 10.8 | 89.2 | 2589 |
| Isotype | 0.45 | 99.6 | 935 | 0.97 | 99 | 915 | 0.67 | 99.3 | 1570 | 6.67 | 93.3 | 50 |

### Evaluation of the Cytotoxicity of the VHHs

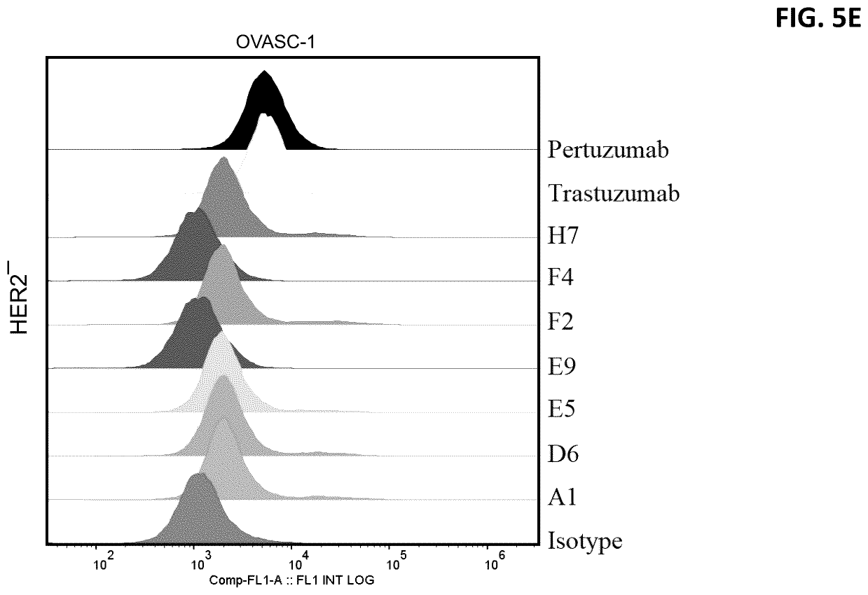

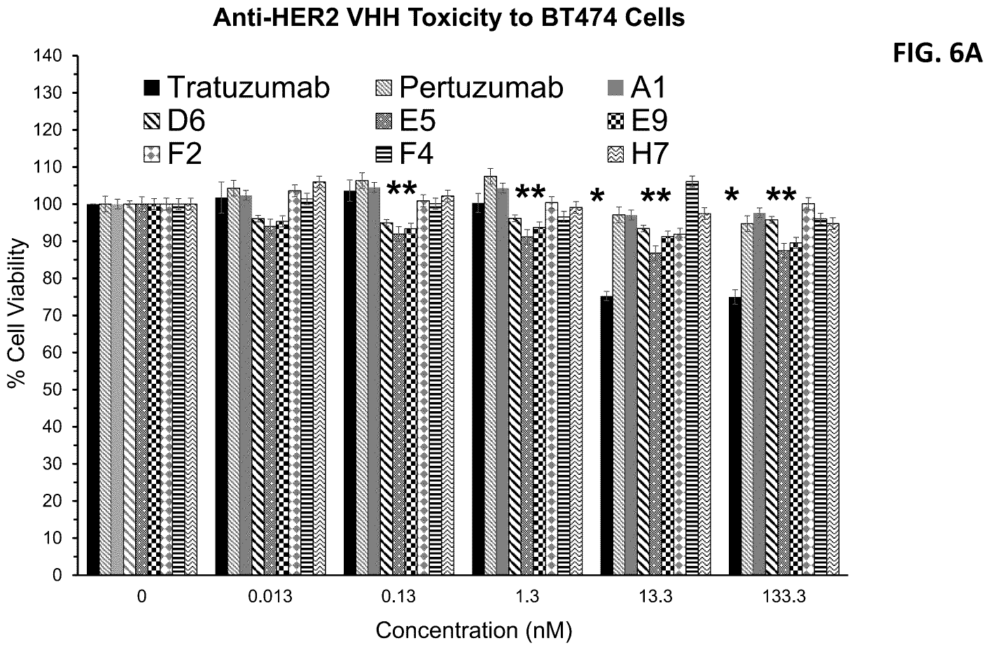

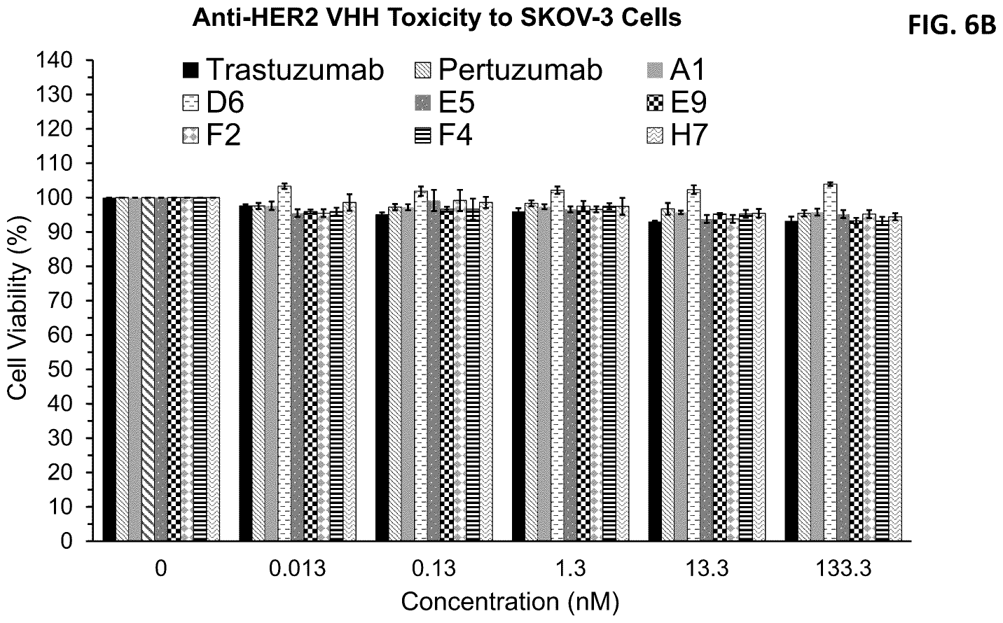

To determine whether the selected VHHs induce toxicity to HER2+ cells, a cell toxicity assay was performed using BT474 and SKOV-3 cancer cells. Trastuzumab and Pertuzumab were used as controls. The results of this experiment revealed that the E5 and A1 clones, similar to Trastuzumab and Pertuzumab (FIGS. 6A-6B), did not induce statistically significant toxicity to SKOV-3 HER2+ cancer cells even at concentration as high as 133 nM making them useful agents for cell imaging. However, BT474 cells appeared to be slightly sensitive to E5 and E9 VHHs with approximately 5% drop in viability (*t-test, p<0.05) (FIGS. 6A-6B). This suggests the application of these two clones in antibody-drug conjugates (ADCs) and also construction of bifunctional VHHs for Antibody-Dependent Cellular Cytotoxicity (ADCC).

### Measurement of VHH Affinity Using Biolayer Interferometer (BLI)

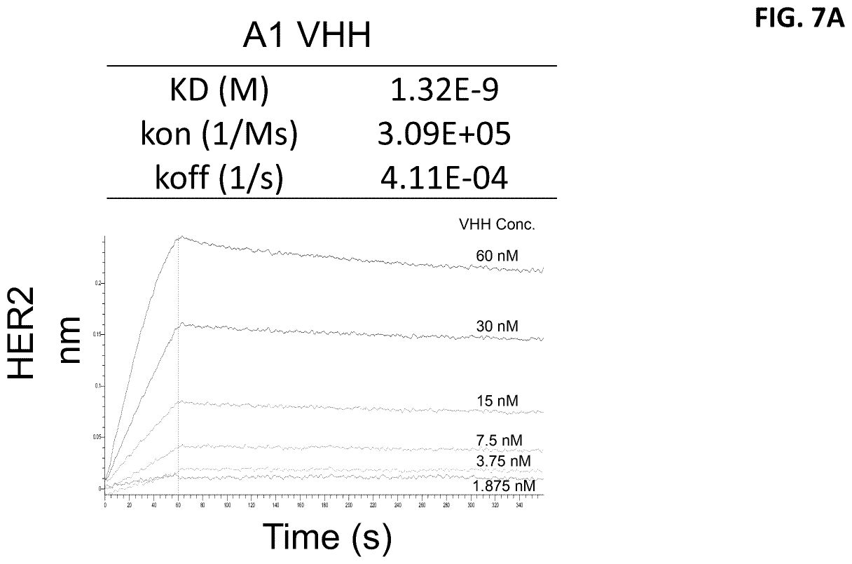

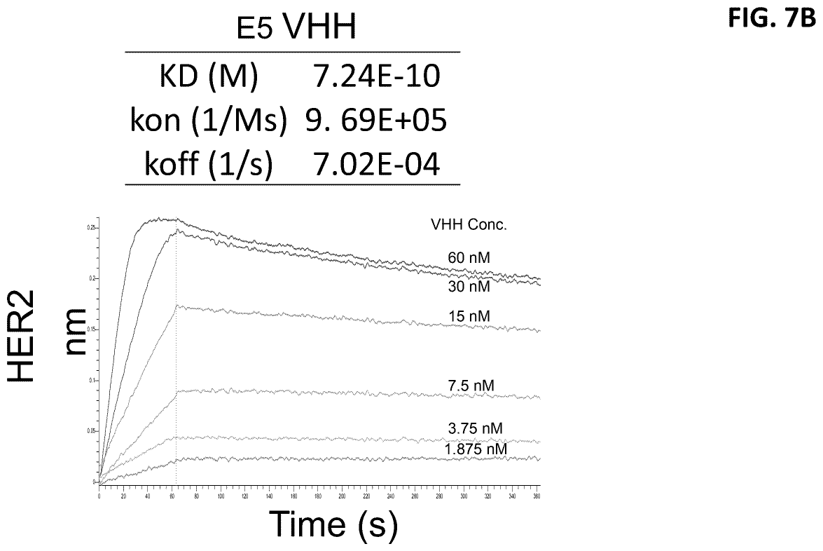

Based on the MFI data in Table 10 and the cytotoxicity data shown above, E5 and A1 clones were selected as the top candidates and characterized them by BLI to measure their affinities towards HER2 antigen. The BLI data showed that the affinities of A1 and E5 clones toward HER2 were 1.3 nM and 724 pM, respectively (FIGS. 7A-7B). Overall, the BLI data show that the E5 and A1 clones are of very high affinity and after binding to HER2 do not dissociate.

### Evaluation of the Application of the VHHs in Cell Imaging

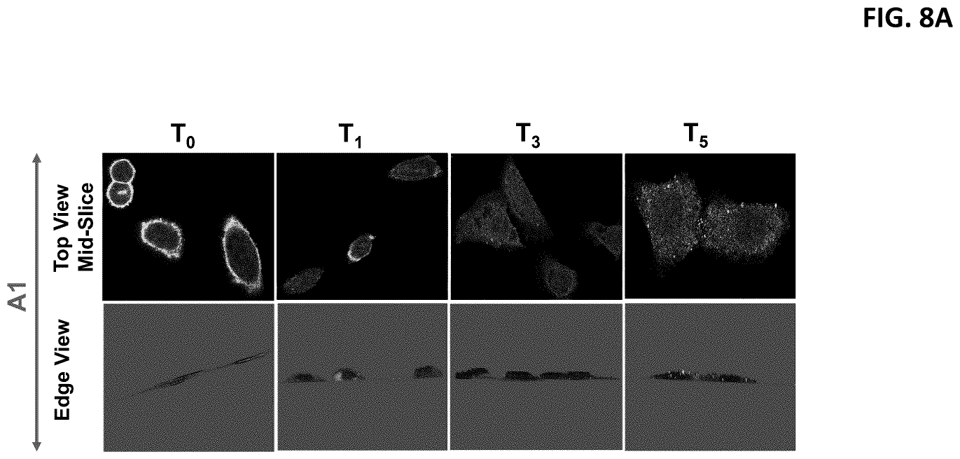

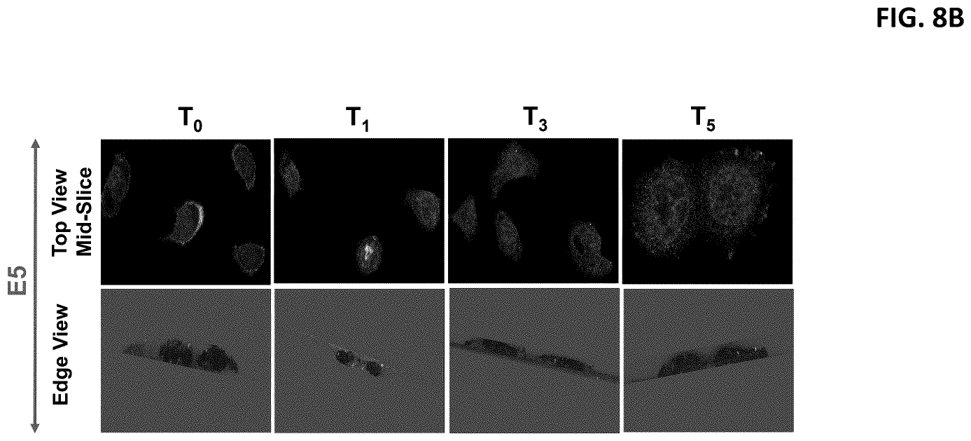

Learning that the selected VHHs were not toxic to cells, their application as a tool for cell imaging was evaluated. For this purpose, SKOV-3 cells were seeded and the binding and internalization of the VHHs into the cells were studied over time by a confocal microscope. The results of this experiment showed that both A1 and E5 clones started to internalize as early as 1 hour and the internalization process completes in 3 to 5 hours (FIGS. 8A-8B). Published data show that Trastuzumab binds and internalizes into HER2+ cancer cells as early as 4 hours (13). These results show the application of the developed VHHs to image HER2+ positive cells.

Overall, the above examples demonstrate the effectiveness of the present single-domain antibodies—including anti-HER2 VHHs—in various imaging and assay methods. HER2 is a receptor that is frequently overexpressed on a variety of aggressive solid tumors and their metastatic lesions, and as a result, it has become a suitable target for preclinical and clinical studies. The present application and foregoing examples demonstrate that the developed anti-HER2 VHH constructs of the present application are able to bind to HER2+ cancer cells with high affinity and specificity with negligible interaction with HER2⁻ cells.

As the present VHHs are constructed with c-myc and histag in their sequences, the present VHHs can be used in flow cytometry, ELISA, cell imaging, and immunohistochemistry methods, and are suitable for ex-vivo cancer phenotyping. Considering that the present VHHs are highly stable and can be made in E. coli, they provide a low-cost alternative to mAbs that are mainly generated and isolated from mammalian cells and animals.

### Examples—Section 2

### Materials and Methods

Below are materials used for the generation, isolation, and characterization of VHHs in the following examples. The list of cells, antibodies and reagents used in the following examples are listed in Table 11.

TABLE 11
The list of the materials used to generate, isolate, and characterize CD16a VHH.

| Item | Vendor | Catalogue No. |
|---|---|---|
| Ni-NTA Agarose | Qiagen (MD, USA) | 30230 |
| FreeStyle ™ 293-F Cells | Gibco ™ (NY, USA) | R79007 |
| FreeStyle ™ 293 Expression Medium | Gibco ™ (NY, USA) | 12338018 |
| Anti-Clumping Agent (1000X) | Gibco ™ (NY, USA) | 0010057AE |
| Trypan Blue Solution, 0.4% | Gibco ™ (NY, USA) | 15250061 |
| Fetal Bovine Serum | Gibco ™ (NY, USA) | 10437028 |
| QIAprep Spin Miniprep Kit | Qiagen (MD, USA) | 27106 |
| QIAquick PCR Purification Kit | Qiagen (MD, USA) | 28106 |
| QIAquick Gel Extraction Kit | Qiagen (MD, USA) | 28704 |
| RNase-Free DNase Set | Qiagen (MD, USA) | 79254 |
| RNeasy Mini Kit | Qiagen (MD, USA) | 74104 |
| QIAGEN Plasmid Mega Kit | Qiagen (MD, USA) | 12181 |
| SuperScript ™ IV First-Strand Synthesis | Thermo Scientific ™ (MA, | 18091050 |
| System | USA) | |
| SYBR ™ Safe DNA Gel Stain | Thermo Scientific ™ (MA, | S33102 |
| | USA) | |
| UltraPure ™ DNA Typing Grade ™ TAE | Thermo Scientific ™ (MA, | 24710030 |
| Buffer | USA) | |
| RIPA Lysis and Extraction Buffer | Thermo Scientific ™ (MA, | 89901 |
| | USA) | |
| DPBS, no calcium, no magnesium | Thermo Scientific ™ (MA, | 14190250 |
| | USA) | |
| AEBSF Protease Inhibitor | Thermo Scientific ™ (MA, | 78431 |
| | USA) | |
| Nuclease-Free Water (not DEPC-Treated) | Thermo Scientific ™ (MA, | AM9937 |
| | USA) | |
| MicroAmp ™ Fast Reaction Tube with Cap | Thermo Scientific ™ (MA, | 4358297 |
| | USA) | |
| Abgene ™ 96 Well Deepwell Storage Plate | Thermo Scientific ™ (MA, | AB0932 |
| | USA) | |
| Ventilating Adhesive Plate Seals | Thermo Scientific ™ (MA, | AB0718 |
| | USA) | |
| S.O.C. Medium | Invitrogen ™ (MA, USA) | 15544034 |
| Polyethylenimine, Linear, MW 25000 | PolyScience (PA, USA) | 23966-1 |
| Carbenicillin (Disodium) | GoldBio (MO, USA) | C-103-25 |
| Isopropyl β-d-1-thiogalactopyranoside | Teknova (CA, USA) | I3325 |
| (IPTG) | | |
| Histopaque ®-1077 (Ficoll) | Sigma-Aldrich (MA, USA) | 10771-500ML |
| Trypsin-EDTA solution | MiliporeSigma (MA, USA) | T4049-100ML |
| D-(+)-Glucose | MiliporeSigma (MA, USA) | G8270-1KG |
| VCSM13 Interference-Resistant Helper | Integrated Science (VA, USA) | 200251 |
| Phage | | |
| Gene Pulser ®/MicroPulser ™ | Bio-Rad (PA, USA) | 1652083 |
| Electroporation Cuvettes, 0.1 cm gap | | |
| Precision Plus Protein ™ Dual Color | Bio-Rad (PA, USA) | 1610374 |
| Standards | | |
| ELISA Coating Buffer | Bio-Rad (PA, USA) | BUF030C |
| Sucrose | Sigma-Aldrich (MA, USA) | S7903-1KG |
| Ethylenediaminetetraacetic acid disodium | Sigma-Aldrich (MA, USA) | E5134-100G |
| salt dihydrate | | |
| 1-Step ™ Turbo TMB-ELISA Substrate | Thermo Scientific ™ (MA, | 34022 |
| Solution | USA) | |
| Escherichia coli (Migula) WK6 | ATCC (VA, USA) | ATCC 47078 |
| Terrific Broth | IBI Scientific (IA, USA) | IB49141 |
| LB Miller Broth | IBI Scientific (IA, USA) | IB49030 |
| Miller's LB Agar | IBI Scientific (IA, USA) | IB49100 |
| Nunc MaxiSorp ™ flat-bottom | Invitrogen ™ (MA, USA) | 44-2404-21 |
| Milk powder, non-fat (skimmed milk), | VWR (PA, USA) | 97063-958 |
| Powder | | |
| WST-1 Cell Proliferation Reagent | Abcam (MA, USA) | 11644807001 |
| Protein LoBind tubes, PCR clean | Eppendorf (MA, USA) | 0030108302 |
| Octet ® Streptavidin (SA) Biosensor | Sartorius (PA, USA) | 18-5019 |
| Nunc ™ Lab-Tek ™ II Chamber Slide ™ | Thermo Scientific ™ (MA, | 154526PK |
| System | USA) | |
| Pierce ™ BCA Protein Assay Kit | Thermo Scientific ™ (MA, | 23225 |
| | USA) | |
| SapI restriction enzyme | NEB (MA, USA) | R0569L |
| Q5 ® High-Fidelity DNA Polymerase | NEB (MA, USA) | M0491S |
| Q5 ® High-Fidelity PCR Kit | NEB (MA, USA) | E0555S |
| Deoxynucleotide (dNTP) Solution Mix | NEB (MA, USA) | N0447S |
| Quick-Load ® Purple 100 bp DNA Ladder | NEB (MA, USA) | N0551S |
| Gel Loading Dye, Purple | NEB (MA, USA) | B7024S |
| T4 DNA Ligase | NEB (MA, USA) | M0202L |
| TrackIt ™ 100 bp DNA Ladder | Thermo Scientific ™ (MA, | 10488058 |
| | USA) | |
| UltraPure ™ Agarose | Thermo Scientific ™ (MA, | 16500100 |
| | USA) | |
| 2x YT media | Sigma-Aldrich (MA, USA) | Y2377-250G |
| Polyethylene glycol | Sigma-Aldrich (MA, USA) | 81260-1KG |
| PlatinumTM II Hot-Start Green PCR Master | Thermo Scientific ™ (MA, | 14001012 |
| Mix | USA) | |
| TG1 Electrocompetent Cells | Lucigen (WI, USA) | 60502-2 |
| Goat Anti-Rabbit IgG H&L (FITC) | Abcam (MA, USA) | ab6717 |
| Anti-HA tag antibody (HRP) | Abcam (MA, USA) | ab1190 |
| Llama IgG-heavy and light chain Antibody | Bethyl (TX, USA) | A160-100P |
| Histag Polyclonal Antibody, DyLight 680 | Thermo Scientific ™ (MA, | 600-444-382 |
| | USA) | |
| Fetal Bovine Serum, qualified, United States | Gibco ™ (NY, USA) | 26140079 |
| Penicillin-Streptomycin (10,000 U/mL) | Gibco ™ (NY, USA) | 15140122 |
| Biotinylated Human Fc gamma RIIIA/ | Acrobiosystems (DE, USA) | CDA-H82E8-25ug |
| CD16a (F176) Protein, Avitag ™, His Tag | | |
| (SPR & BLI verified) | | |
| Biotinylated Human Fc gamma RIIIB/ | Acrobiosystems (DE, USA) | CDB-H82E4-25ug |
| CD16b (NA1) Protein, His,Avitag ™ (SPR | | |
| & BLI verified) | | |
| Biotinylated Human Fc gamma RIIIB/ | Acrobiosystems (DE, USA) | CDB-H82Ea-25ug |
| CD16b (NA2) Protein, His, Avitag ™ (SPR | | |
| & BLI verified) | | |
| MEM α, no nucleosides | Gibco ™ (NY, USA) | 12561056 |
| Fetal Bovine Serum, qualified, United States | Gibco ™ (NY, USA) | 26140079 |
| Horse Serum, New Zealand origin | Gibco ™ (NY, USA) | 16050122 |
| myo-Inositol | Sigma-Aldrich (MA, USA) | I7508-100G |
| Folic Acid | Sigma-Aldrich (MA, USA) | F8758-5G |
| Penicillin-Streptomycin (10,000 U/mL) | Gibco ™ (NY, USA) | 15140122 |
| Acetic acid (17.48 M) | Sigma-Aldrich (MA, USA) | A6283-100ML |
| DPBS, no calcium, no magnesium | Gibco ™ (NY, USA) | 14190144 |
| Bovine Albumin Fraction V (7.5% solution) | Gibco ™ (NY, USA) | 15260037 |
| Hydrochloric acid solution | Sigma-Aldrich (MA, USA) | H9892-100ML |
| Animal-Free Recombinant Human IL-2 | Peprotech (NJ, USA) | AF-200-02-250UG |
| 2-Mercaptoethanol | Gibco ™ (NY, USA) | 21985023 |
| 0.2 μm Syringe filters with acrylic housing | VWR (PA, USA) | 28145-501 |
| Genetically modified natural killer (NK- | ATCC (VA, USA) | PTA-8836 |
| 92 ®) cell Line retroviral transduced to | | |
| express Human CD16 | | |
| Neutrophils | HemaCare (CA, USA) | PB011C-1 |
| PE Mouse Anti-Human CD16b | BD Bioscience (NJ, USA) | 550868 |
| RPMI-1640 Medium | Sigma-Aldrich (MA, USA) | R8758-6X500ML |
| CD16 Monoclonal Antibody (3G8), | Thermo Scientific ™ (MA, | 16-0166-82 |
| Functional Grade, eBioscience ™ | USA) | |

### Expression and Purification of CD16a Protein

The gene encoding CD16a ectodomain (UniProt ID P08637) was synthesized by VectorBuilder (IL, USA) and then cloned into a mammalian expression vector under EF-1a promoter. The mammalian expression vector was selected as the CD16a ectodomain is a glycosylated protein. A secretory signal sequence was designed at the gene's N-terminal and a 12xhistag (SEQ ID NO: 2) at its C-terminal to facilitate purification of the protein from the culture media by Ni-NTA column chromatography (Table 12). To express CD16a, FreeStyle™ 293-F cells were cultured as per manufacture's recommendations and then seeded at 4×10⁵ cells/ml 250 mL Reusable Spinner. Cells were passaged every three days. On the day of transfection, cells were seeded at ˜3×10⁶ cells/ml in 100 ml of FreeStyle™ 293 expression media using 250 mL Reusable Spinner Flask for 30 min. The CD16a expression vector was then complexed with polyethylenimine (PEI). For every 10⁶ cells, 1 μg plasmid was mixed with 4 μg of PEI (1:4 w/w ratio) in Opti-MEM I and incubated at room temperature (RT) for 20 minutes. Next, the plasmid: PEI complexes were added to the seeded cells dropwise under constant stirring. The flask was incubated at 37° C. with 5% CO2 for 24 h. The next day, two-fold fresh FreeStyle media was added to the transfected cells. Protein expression continued for 8 to 10 days, or until the viability dropped below 85%, whichever came first. The downstream process started by harvesting the cells using 10,000 g, 10 min, 4° C. The supernatant was collected and filtered through a 0.45 μm filter to remove the cell debris. Next, 300 μL of Ni-NTA resin was washed with equilibration buffer (500 mM NaCl, 20 mM Na2HPO4, 50 mM Tris, pH 7.4), mixed with the supernatant and then incubated at 4° C. overnight. The next day, the supernatant: Ni-NTA mixture was poured into a chromatography column and washed using 20 mL of washing buffer (500 mM NaCl, 20 mM Na2HPO4, 50 mM Tris, 7.5 mM imidazole, pH 7.4). Finally, recombinant CD16a (rCD16a) protein was eluted using 500 μL of elution buffer (500 mM NaCl, 20 mM Na2HPO4, 50 mM Tris, 250 mM imidazole, pH 7.4). The purity and molecular weight of the purified protein were estimated by SDS-PAGE.

TABLE 12
The amino acid sequence of the secretory cd16 protein cloned into eCD16bac
plasmid with theoretical molecular weight of 24.56 kDa

| Name | Amino Acid Sequence |
|---|---|
| rCD16a | METDTLLLWVLLLWVPGSTGDAAQPARRASLWELQGMRTE |
| (Ectodomain) | DLPKAVVFLEPQWYRVLEKDSVTLKCQGAYSPEDNSTQWFH |
| | NESLISSQASSYFIDAATVDDSGEYRCQTNLSTLSDPVQLEVHI |
| | GWLLLQAPRWVFKEEDPIHLRCHSWKNTALHKVTYLQNGKG |
| | RKYFHHNSDFYIPKATLKDSGSYFCRGLFGSKNVSSETVNITIT |
| | QGLAVSTISSFFPPGYQWELQLEEGPHHHHHHHHHHHH* (SEQ |
| | ID NO: 26) |

*Secretory signal (BOLD) - rCD16a ectodomain - Protease site (italics) Histag (HHHHHHHHHHHH (SEQ ID NO: 2))

Immunization of llama with CD16a Protein

Llama immunization was carried out by Capralogics Inc. (Gilbertville, MA). A female llama was immunized via six injections every two weeks using rCD16a (500 μg/injection). The antigen of interest was mixed with either Complete (first injection) or incomplete Freund's adjuvant (second, third and fifth injections) to maximize the immune response. The fourth and sixth injections were performed using rCD16a without any adjuvant. After the fourth injection, 50 mL of whole blood was collected, and the immunization was confirmed using ELISA using commercial CD16a (Acrobiosystem, CDA-H82E9-25 ug). After immunization confirmation, llama immunization was continued for ten more weeks. Five days following the sixth injection, 600 mL of whole blood was withdrawn to isolate the peripheral blood mononuclear cell (PBMC).

### Isolation of Peripheral Blood Mononuclear Cells (PBMCs)

First, the whole blood was diluted twofold using DPBS-3% FBS. Diluting serum significantly improves PBMC recovery. Next, PBMCs were isolated using density gradient centrifugation. Afterward, 35 mL of diluted serum was gently layered on Ficoll (Histopaque®-1077) not to disrupt the interphase. Subsequently, the tube was centrifuged using a swinging—bucket rotor at 400×g for 45 min with brake off. The upper layer was removed very gently. Then PBMCs were collected from undisturbed interphase. The collected PBMCs were transferred into a new 50 mL tube and immediately washed to remove the remnant of Ficoll, which is toxic to the cells.

### Library Generation

After collecting and counting the cells using the Trypan Blue Exclusion method, total RNA was extracted using RNeasy Mini Kit followed by cDNA synthesis using oligo-dT and random hexamer via SuperScript™ IV First-Strand Synthesis System. Afterwards, the generated cDNA was used as a template in the Nested Polymerase chain reaction (PCR) reaction using primers in Table 13 and PCR protocol in Table 14. The first round of PCR was performed using CALL001 and CALL002. The product was run on 1% agarose gel, and the 700 bp band was excised and purified. Next, the first round of PCR products was applied in the second round of PCR using VHH-Back and VHH-Forward. The 400 bp band in agarose gel was excised, purified, and cloned into pMECS-GG phagemid (Kindly provided by Dr. S. Muyldermans, Belgium) using the Golden Gate cloning system. Finally, the recombinant phagemid was used to transform TG1 E. coli (Electroporator setting: 2.5 kV, 25 μF, 200Ω. Ten vials of competent TG1 were transformed to keep the diversity of the library. Then 1 ml of SOC medium was added and transferred into a 50 ml tube. After 60 min incubation at 37° C., the bacteria were harvested at 6,000×g for 15 min. The transformants were selected in six flasks of 100 mL Luria-Bertani (LB) supplemented with 100 μg/mL Carbenicillin. The propagated bacteria were spun down and resuspended in 20 mL of fresh LB media. After adding 15% glycerol, the library was kept in a −80° C. freezer.

TABLE 13
The list of primers and the corresponding sequences used to make VHH cDNAs.

| Name | Sequence |
|---|---|
| CALL001 | 5′- GTCCTGGCTGCTCTTCTACAAGG-3′ (SEQ ID NO: 22) |
| CALL002 | 5′- GGTACGTGCTGTTGAACTGTTCC-3′ (SEQ ID NO: 23) |
| VHH-Back | 5′- GATGGGCTCTTCTGTGTGCAGCTGCAGGAGTCTGGRGGAGG-3′ |
| | (SEQ ID NO: 24) |
| VHH- | 5′-CTAGTGCTCTTCCGCTTGGAGACGGTGACCTGGGT-3′ |
| Forward | (SEQ ID NO: 25) |

TABLE 14
The PCR protocol for the amplification of the primers.

| Cycle | Step | Temperature | time |
|---|---|---|---|
| 1 X | Initial | 95 C. | 3 min |
| | Denaturation | | |
| 35 X | Denaturation | 95 C. | 1 min |
| | Annealing | 50-60 C. | 1 min |
| | Synthesis | 72 C. | 1 min |
| 1 X | Final Extension | 72 C. | 10 min |
| 1 X | | 4 C. | — |

### Phage Display

Four rounds of phage displays were performed to reach a specific CD16a library. First, 2 OD600 nm (1 OD₆₀₀ₙₘ=2.66×10⁹ cells/ml) of llama library was inoculated into 100 mL of LB supplemented with 100 μg/mL Carbenicillin. Upon the OD reached 0.6, 500 μL of the helper phage, VCSM13, was added to the culture and incubated at 37° C. without shaking for 60 min. Next, 50 μg/mL Kanamycin was added to the culture and incubated at 37° C. overnight with shaking. The next day, the bacteria were spun down using 10,000×g, 20 min, 4° C., and 10% PEG-NaCl was added to the supernatant and kept on ice for 60-120 minutes. Finally, the recombinant phages were recovered via centrifugation (3,000×g, 20 min, at 4° C.) followed by washing three steps using DPBS.

The purified phages were used to bind to CD16a protein in polyclonal phage ELISA. In this experiment, all the incubations were at RT and shaking speed 700 rpm unless mentioned otherwise. To start the panning, 100 μL of Streptavidin (5 μg/mL) was coated in a 96-well plate overnight at 4° C. The next day, the coated plate was washed once using DPBS and blocked using 2% skimmed milk. Next, 50 μL of biotinylated CD16a (1 μg/mL) was added and incubated at 45 min at RT while shaking, followed by washing three steps using DPBS, 0.1% Tween 20 (0.1% DPBS-T). Then, around 100 μL of recombinant phages were added. Afterward, to remove non-specific VHHs, the plate was washed ten times with 0.1% DPBS-T followed by ten steps washing using DPBS. Finally, the binders were recovered using 50 μL of 0.25% Trypsin. Following neutralizing Trypsin via 4-(2-Aminoethyl)benzene-1-sulfonyl fluoride (AEBSF), 100 μL of phages were used to infect 100 μL of TG1 for 1 h at 37° C. Then, 100 μL of infected TG1 was transferred into 100 mL LB media containing 100 μg/mL Carbenicillin, and bacterial culture continued overnight.

### Preparation of Periplasmic Extract and Evaluation of Affinity and Specificity by ELISA

To select the top binders, 190 colonies were randomly selected to be cultured in 1 mL LB supplemented with 100 μg/mL Carbenicillin and 2% Glucose in a Deep-Well 96-well plate. To keep the culture oxygenated, ventilating adhesive plate was used to cover the plate. The following day, a fresh 1 mL LB supplemented with 100 μg/mL Carbenicillin was inoculated with 10 μL of overnight culture. After 4 h, the expression was induced using 1 mM Isopropyl β-d-1-thiogalactopyranoside (IPTG). The expression continued overnight at 28° C. The next day, the bacteria were harvested using centrifugation (10,000×g, 7 min, 4° C.). The supernatants were discarded, and the pellet was stored at −20° C. Next, the periplasmic extract was prepared using three cycles of freeze-thaw (30 min at −20° C., 10 min at RT). Next, 500 μL of DPBS was added to the pellet and placed on a shaker for 30 min. Finally, the supernatants (400 μL) were transferred into a new Deep-Well 96-well plate following centrifugation (10,000×g, 30 min, 4° C.). The periplasmic extract was used in ELISA to find the top candidate.

The periplasmic extract was used in ELISA to find the best binders. In this experiment, all the washings were performed using 0.1% DPBS-T, incubation times were 60 min, incubations at RT and shaking speed was 700 rpm unless mentioned otherwise. After each antibody, the plate was washed six times to remove all the weak binders. First, the plate was coated using 100 μL of 5 μg/mL Streptavidin overnight at 4° C. The next day, after one step of washing, 50 μL of biotinylated CD16a and CD16b was added and incubated for 30 minutes. After three steps of washing, 100 μL of the periplasmic extract was added. To detect the bound VHH, the secondary antibody, HRP Anti-HA tag antibody (1:10000 dilution), was added. To develop the color, 50 μL of 1-Step™ Turbo TMB-ELISA substrate solution was added and incubated in darkness for 15 min. Following stopping the reaction, the plate was read at 450 nm wavelength. In order to narrow down the top candidate to two, the serial dilution of the periplasmic extract of the top 20 VHHs was used in ELISA, as mentioned above. The top two candidates (C1 and E3 clones) were selected based on the highest OD in the highest dilution.

### Expression and Purification of Selected VHHs

The selected VHHs were sequenced and then cloned into an expression plasmid vector. The plasmids were then chemically transformed into WK6 E. coli. First, the highest expressing transformant was selected by western blot. Next, 750 mL Terrific Broth (TB) supplemented with 0.1% Glucose, 1 mM MgCl2 and 100 μg/mL carbenicillin was inoculated by 7.5 mL of overnight culture. After the OD600 nm of 0.6-0.8 was obtained, 0.5 mM IPTG was added to start the expression. The culture was then incubated at 28° C. overnight. The next day, the bacterial cells were spun down using a centrifuge (10,000×g, 10 min, 4° C.). Then the pellets were resuspended in 10 mL of TES buffer (200 mM Tris-HCl, pH 8.0, 500 mM sucrose, 1 mM EDTA) and incubated at 4° C. for 1 h while shaking. Afterward, 15 mL of TES/4 buffer (10 mL TES buffer was added to 30 ml distilled water) was added and incubated at 4° C. for 45 min while shaking. Next, the bacterial cells were pelleted via centrifugation (40,000×g, 4° C., 30 min). After that, the supernatant was loaded onto the Ni-NTA column. The non-specific proteins were washed out by 30 mL of wash buffer (500 mM NaCl, 20 mM Na2HPO4, 50 mM Tris, 25 mM imidazole, pH 7.4). The purified protein was eluted using elution buffer (500 mM NaCl, 20 mM Na2HPO4, 50 mM Tris, 250 mM imidazole, pH 7.4). The quality and quantity of the eluted protein were evaluated by SDS-PAGE and BCA kit, respectively.

### Evaluation of the VHH Specificity by ELISA

The purified VHHs were subjected to functional analysis using ELISA and flow cytometry. For ELISA, 50 μL of biotinylated CD16a, CD16b-NA1, CD16b-NA2, and CD32b were added to the streptavidin-coated plate and incubated for 45 min at RT. Following washing, the 100 μL of 100 nM purified VHHs were added and incubated at RT for 1 h. Next, 100 μL anti-HA tag HRP conjugated secondary antibody was added. Finally, the substrate was added for color development, and the plate was read using OD of 450 nm.

Evaluation of the Ability of VHHs to Recognize CD16a+ haNK92 Cells by Using Flow Cytometry

For flow cytometry, haNK92 cells (CD16a+) and Neutrophils (CD16b⁺) were used. CD16a+ haNK92 cells were purchased from ATCC and cultured in MEM-α supplemented with 12.5% Fetal Bovine Serum (FBS), 12.5% Horse Serum, 0.2 mM myo-inositol, 0.02 mM Folic Acid, 100 U/ml penicillin-streptomycin, 0.1 mM 2-Mercaptoethanol and 100 U/ml IL-2. The human Neutrophil cells were purchased from HemaCare and cultured according to the manufacturer's protocol. First, both cell lines were harvested and washed once using DPBS supplemented with 2% FBS (2% DPBS-FBS). For each sample, 0.5×10⁶ cells were stained with antibody on ice for 60 min. In this experiment, 3G8 monoclonal antibody was used as a positive control. This antibody binds to both CD16a and CD16b. Following washing three times, the secondary antibody, anti-Histag antibody FITC labeled, was added. After washing, the samples were run on Cytoflex using FL2 channel.

### Measurement of VHH Binding Affinity and Kinetics Using Biolayer Interferometer

To evaluate the binding affinity (KD) and constant rates of association (Kon) and dissociation (Koff) of VHHs, an Octet RED96e instrument (Sartorius) Biolayer Interferometer (BLI) along with an Octet® Streptavidin (SA) Biosensor were used. In this experiment, 3G8 was used as a positive control. After soaking the sensor for at least 10 min in DPBS, biotinylated CD16a, CD16b NA1, and CD16b NA2 were loaded onto the Streptavidin (SA) Biosensor for 10 min. Next, the sensor was dipped into the washing buffer (DPBS+0.05% Tween 20) for 2 min to reach the baseline. Then, the sensor was submerged into wells containing 100, 50, 25, 12.5, 6.25, 3.25, and 0 nM of purified 3G8 mAb or anti-CD16a VHHs for 5 min (association step). Then, in the dissociation step, wells were dipped into washing buffer (DPBS+0.05% Tween 20) for 10 min to acquire data (Table 15). The data were then analyzed using Octet Data Analysis HT 11.1 software. The sensorgrams were subtracted from the reference and fitted into 1:1 and 2:1 binding model for data analysis. Finally, the affinity and kinetics were analyzed using “Association and Dissociation”.

TABLE 15
The experimental conditions used to acquire data from the BLI.

| | | | Shake |
|---|---|---|---|
| Step | Name | Time (s) | Speed |
| 1 | Sensor Check | 30 | 1000 |
| 2 | Loading (10 μg/mL CD16) | 600 | 1000 |
| 3 | Baseline (DPBS + 0.05% Tween 20) | 120 | 1000 |
| 4 | Association | 300 | 1000 |
| 5 | Dissociation (DPBS + 0.05% Tween 20) | 600 | 1000 |

### Results and Discussion

Expression of rCD16a and Immunization of Llama

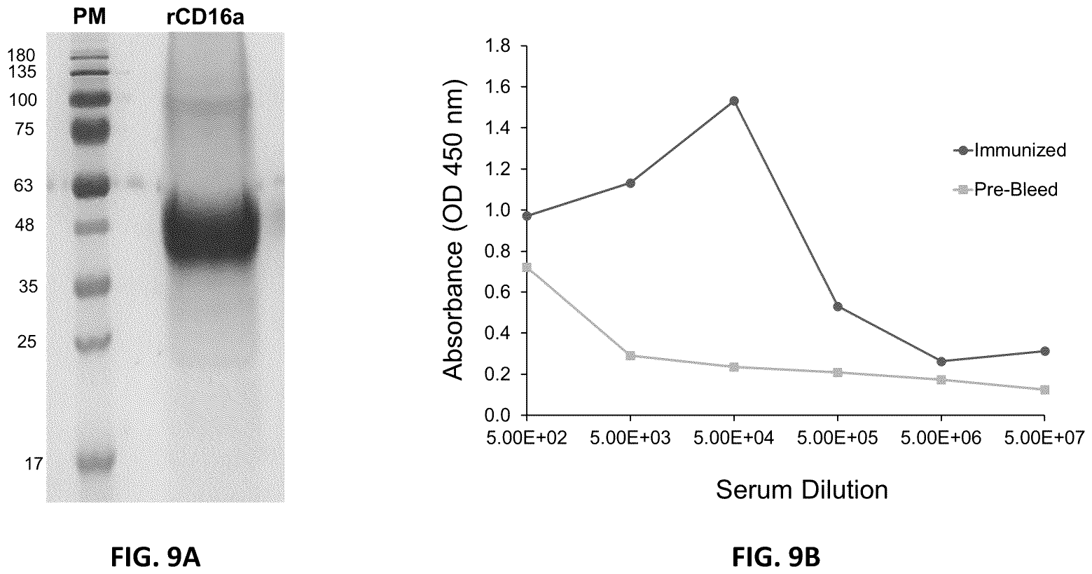

rCD16a was genetically engineered and expressed in 293-F mammalian expression system and then purified. The SDS-PAGE analysis of purified protein revealed rCD16a with >95% purity (FIG. 9A). The observed molecular weight is ˜48 kDa, while the theoretical molecular weight (expected) is 21.83 kDa. The difference between expected and observed molecular weight comes from five glycosylation sites on CD16a ectodomain. Correct glycosylation is vital for protein folding and immunogenicity.

The purified rCD16a was then used to immunize llama. Blood draw four weeks post immunization followed by ELISA showed significant elevation in IgG levels (OD₄₅₀ of 1.6 after 1/50,000 dilution) indicating potent immune response to the injected rCD16a antigen. In this experiment, pre-immunization serum was used as a negative control. As it can be observed, the serum of immunized llama had a high titer of antibody against rCD16a as compared to pre-immunization (FIG. 9B). The data indicates that the concentration of injected rCD16a was sufficient to raise the humoral immune response.

### Isolation of PBMCs, Library Generation, Phage Display, and Colony Screening

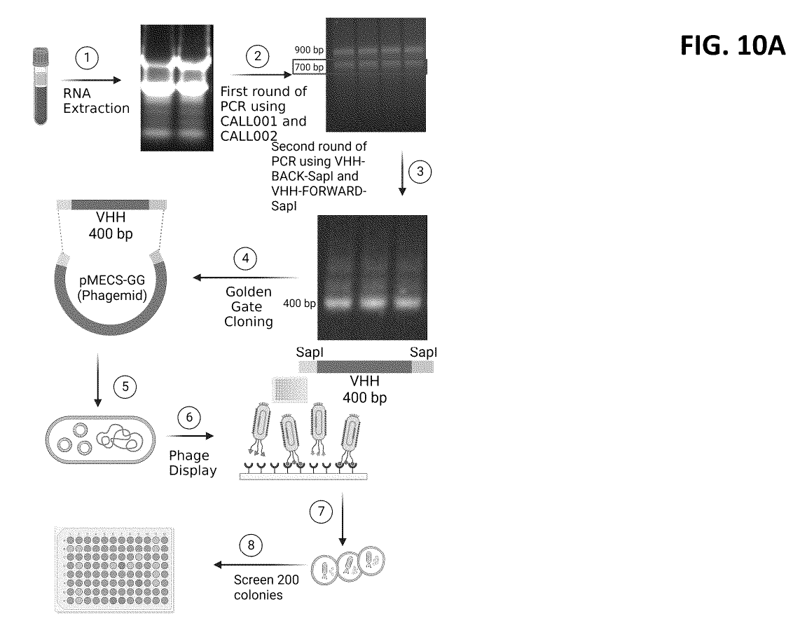

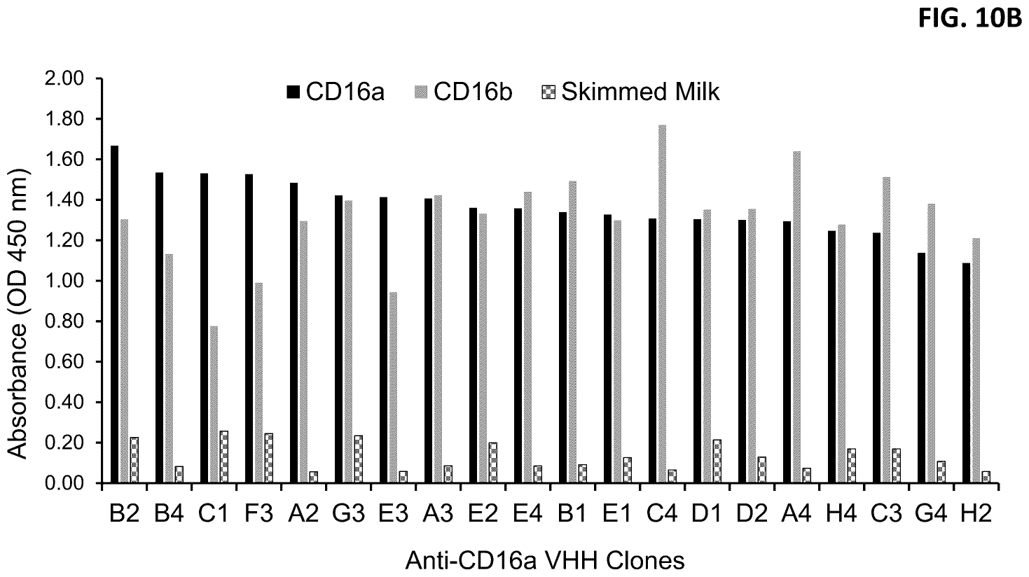

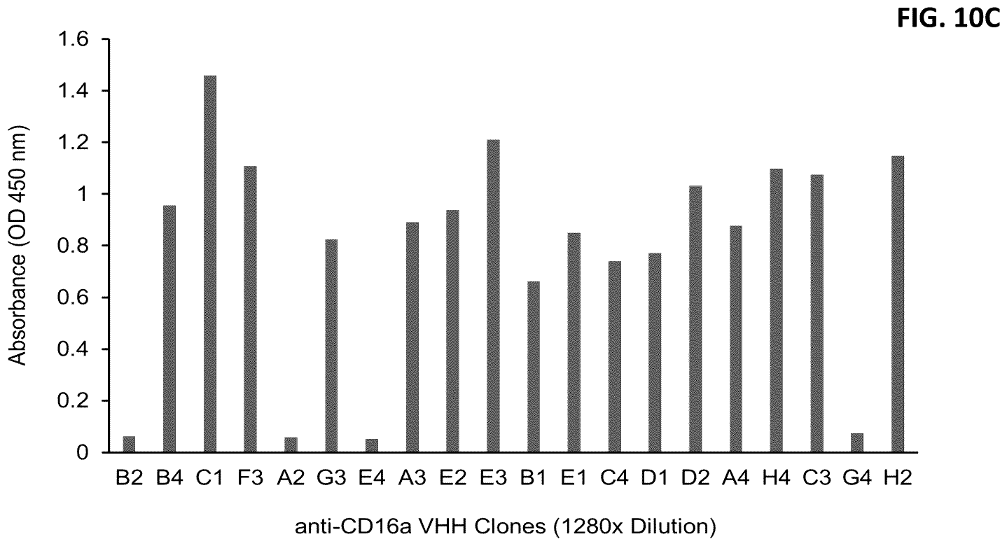

On day 75 post llama immunization, blood was drawn, PBMCs isolated, a cDNA and phagemid library were generated, and then used in phage display. After four rounds of phage display, 20 colonies were selected, and lysed to obtain periplasmic extract. Lysates with the highest binding affinity toward CD16a antigen were selected and then examined for specificity by using ELISA (FIG. 10A). The results of these experiments revealed a few VHH clones with higher binding affinity toward CD16a than CD16b-NA1 antigen (FIG. 10B). To identify the high-performing VHH candidates in terms of binding affinity, a 1280-fold dilution of periplasmic extract was prepared and used to bind to CD16a antigen. The results of ELISA showed that except for A1, A2, E4, and G4 clones, the rest of the VHHs had high binding affinity; however, the C1 and E3 clones appeared to be the highest-performing constructs (FIG. 10C). Therefore, these two VHHs were selected based on their specificity and high affinity for further studies.

### Expression and Purification of C1 and E3 Clones

The phagemids encoding C1 and E3 VHHs were used to transform WK6 E. coli for expression in periplasmic space (Table 16), and then purified. Periplasmic space provides the opportunity for the expressed VHHs to fold properly. The purified VHHs were then analyzed by SDS-PAGE for purity. The results of this experiment showed the purity of both VHHs were >95% and with the yield of 2-3 mg/l of culture media.

TABLE 16
The amino acid sequences of the C1 and E3 VHHs with affinity and specificity
toward CD16a.

| VHH | | | | | | | Framework | |
|---|---|---|---|---|---|---|---|---|
| Code | Framework 1 | CDR1 | Framework 2 | CDR2 | Framework 3 | CDR3 | 4 | Tag |
| C1 | QVQLQESGGGL | GRTFRLY | MGWFRQA | IKMIDGS | LYGDPVKGRFTISRDNT | ASVSRVTGSY | WGQGTQ | HA- |
| (SEQ | VQAGGSLRLSC | R (SEQ ID | PGKEREFV | T (SEQ ID | KFMAYLQMNSLKPEDT | DS (SEQ ID | VTVSS | Tag + |
| ID | AAS (SEQ ID | NO: 29) | GS (SEQ ID | NO: 31) | AVYYC | NO: 33) | (SEQ ID | Histag |
| NO 14) | NO: 28) | | NO: 30) | | (SEQ ID NO: 32) | | NO: 34) | |
| E3 | QVQLQESGGGL | GRTFRLY | MGWFRQA | IKMIDGS | LFGDPVKGRFTISRDNT | ASVSRNTGSW | WGQGTQ | HA- |
| (SEQ | VQAGGSARLSC | R (SEQ ID | PGKEREFV | S (SEQ ID | KFMVYLQMNSLKPEDT | DS (SEQ ID | VTVSS | Tag + |
| ID | AAS (SEQ ID | NO: 29) | AS (SEQ ID | NO: 37) | AVYYC | NO: 39) | (SEQ ID | Histag |
| NO 15) | NO: 35) | | NO: 36) | | (SEQ ID NO: 38) | | NO: 34) | |

### Evaluation of the Specificity of the Purified C1 and E3 VHHs

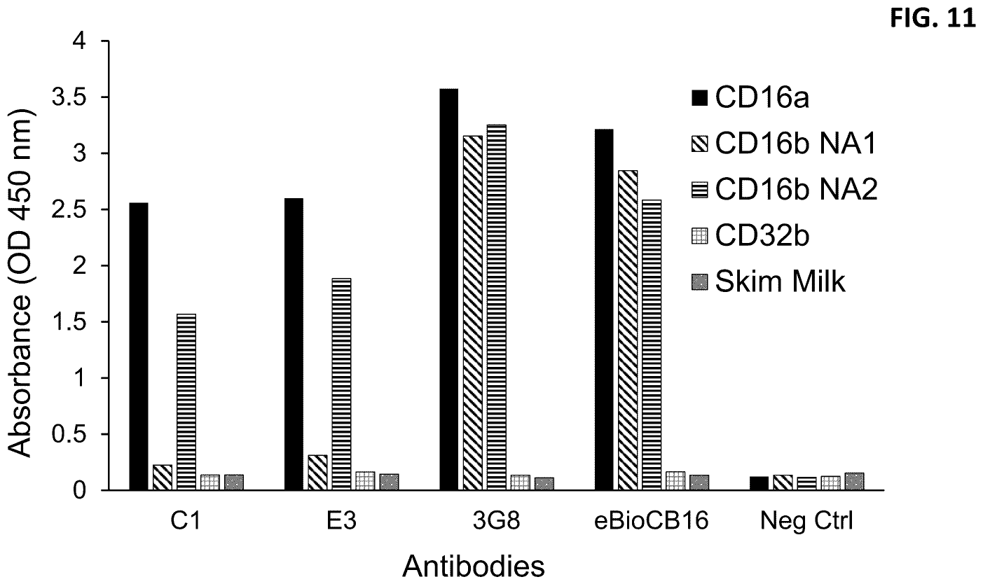

To determine the specificity of the purified C1 and E3 clones toward CD16a, first an ELISA was performed. Since CD16b has two predominant alleles, including human neutrophil antigen 1 (NA1) and NA2 (10), both were used as controls. CD32b and skim milk were also used as antigen controls. 3G8 and eBioCB16, which are anti-CD16a/b mAbs were used as antibody controls. It is noteworthy that a CD16a-selective antibody is not commercially available to be used as a control. The results of ELISA illustrated both C1 and E3 VHHs effectively bound to CD16a antigen without any significant interaction with CD32b antigen (FIG. 11). CD32b is a receptor that is expressed on B cells and responsible for inhibiting B cell activation. One of the disadvantages of mAb-based therapies is the binding of their Fc region to CD32b leading to the diminished activity of humoral immunity. In addition to CD32b, C1 and E3 VHHs did not interact with CD16b-NA1 antigen, but interacted with CD16b-NA2 antigen (FIG. 11).

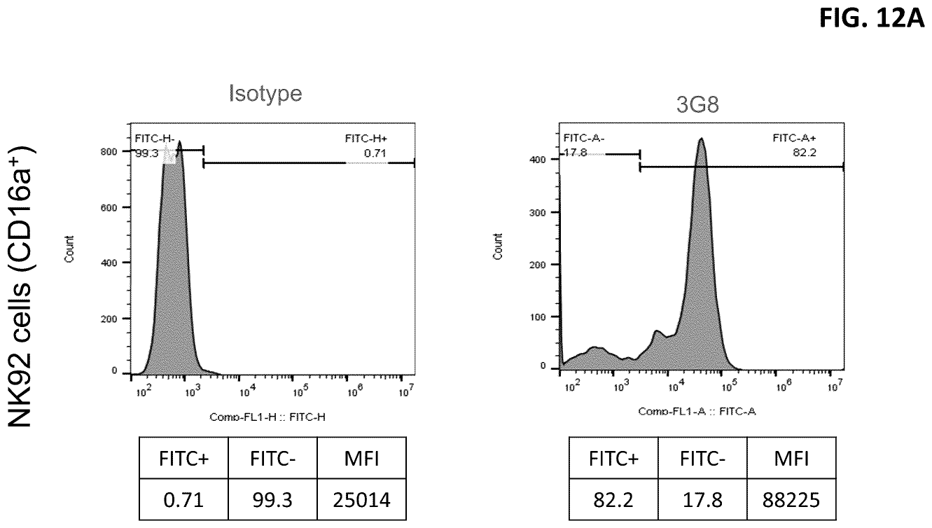

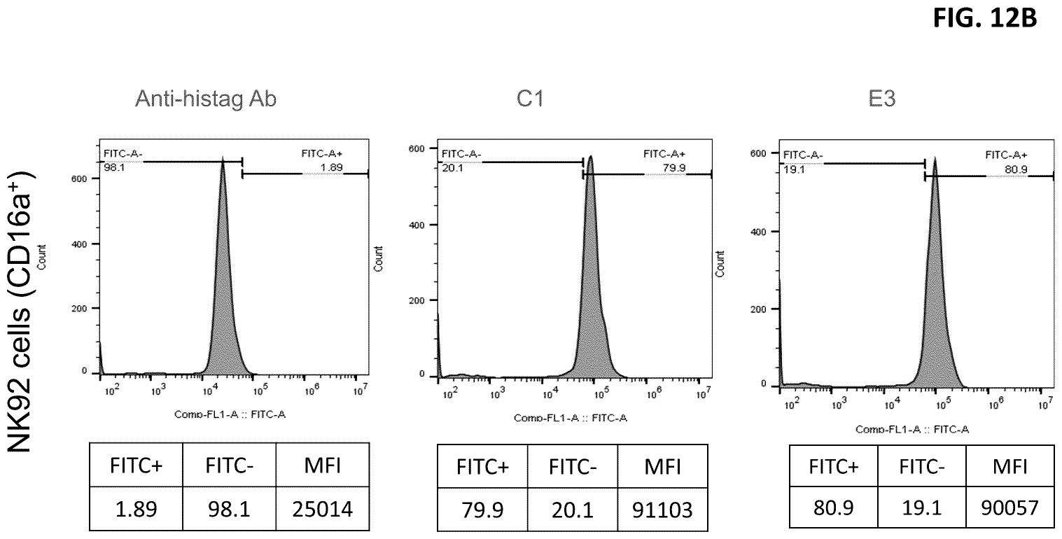

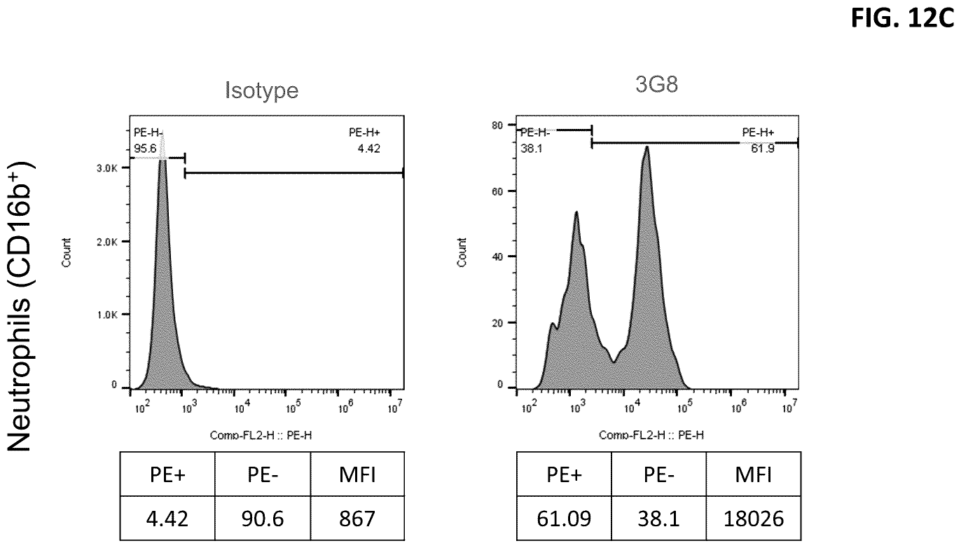

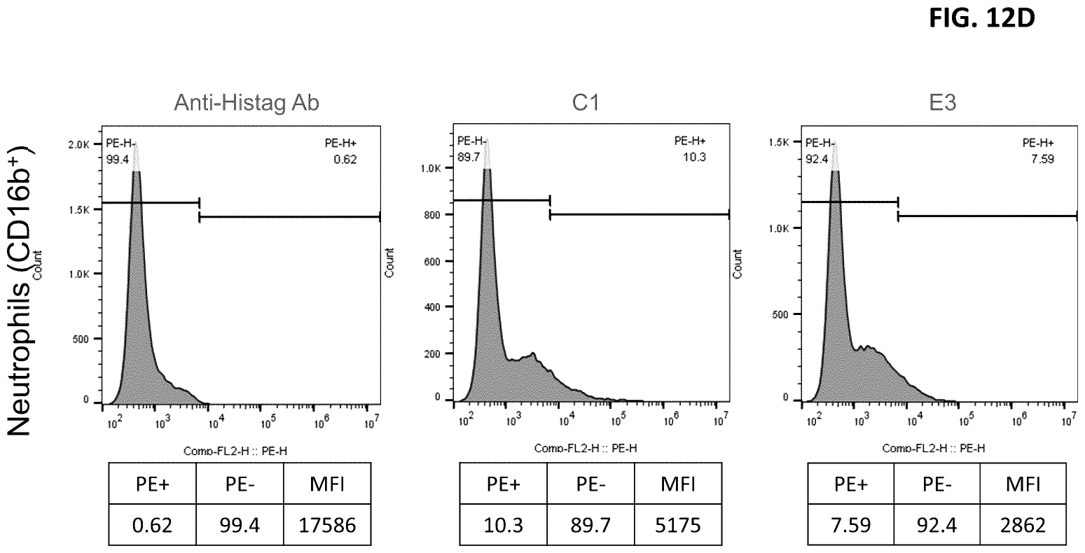

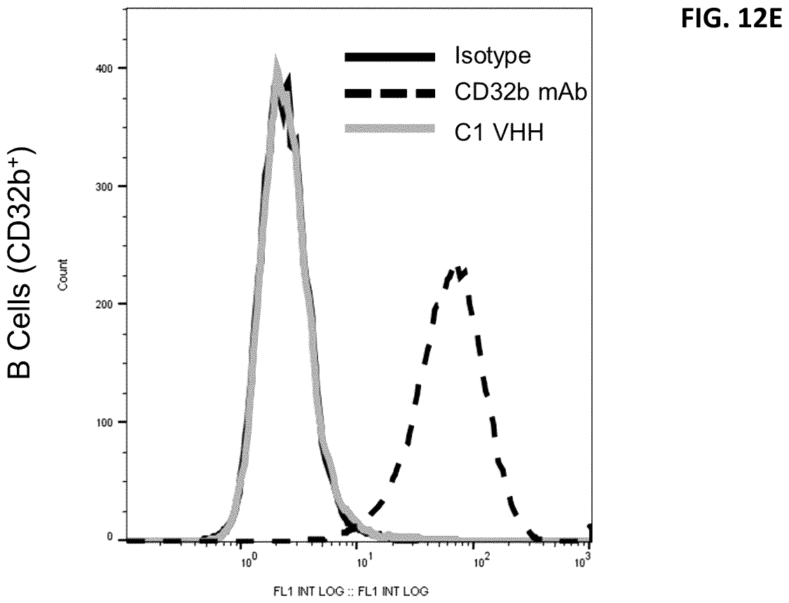

In the next step, the specificity of the C1 and E3 VHHs was evaluated by using flow cytometry. In this experiment, NK92 cells (CD16a⁺), neutrophils (CD16b⁺), and B cells (CD32b⁺) were used as cell controls. 3G8 (anti-CD16a/b) and AT10 (anti-CD32a/b) mAbs were used as antibody controls. The results of this experiment showed that C1 and E3 VHHs effectively bound to NK92 cells but not neutrophils (FIGS. 12A-12D). In addition, the C1 clone did not bind to B cells (FIG. 12E). In contrast, 3G8 mAb bound to both NK92 and neutrophils and AT10 to B cells, non-discriminately. Since C1 and E3 VHHs only bind to NK cells without being drained by neutrophils (4), this outcome has a wide range of clinical applications.

### Measuring the Affinity of VHHs by Using BLI

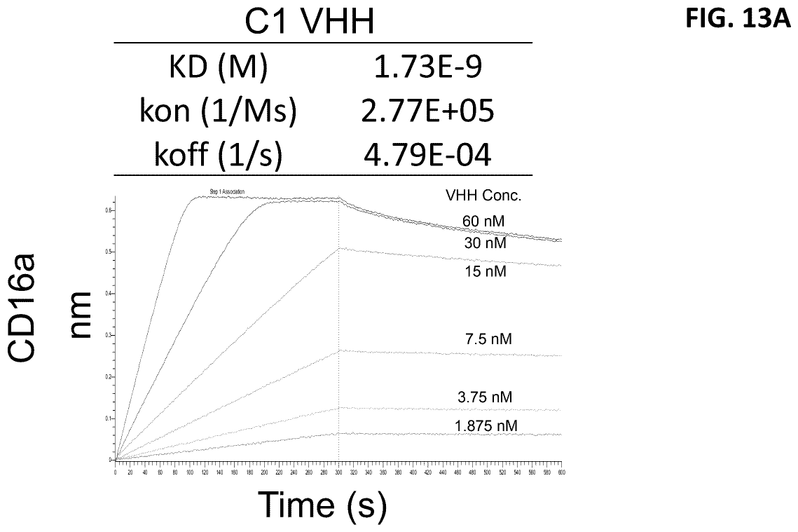

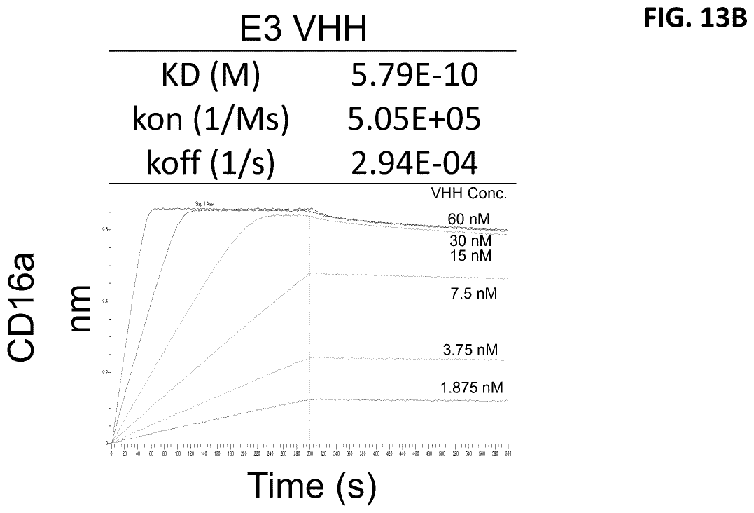

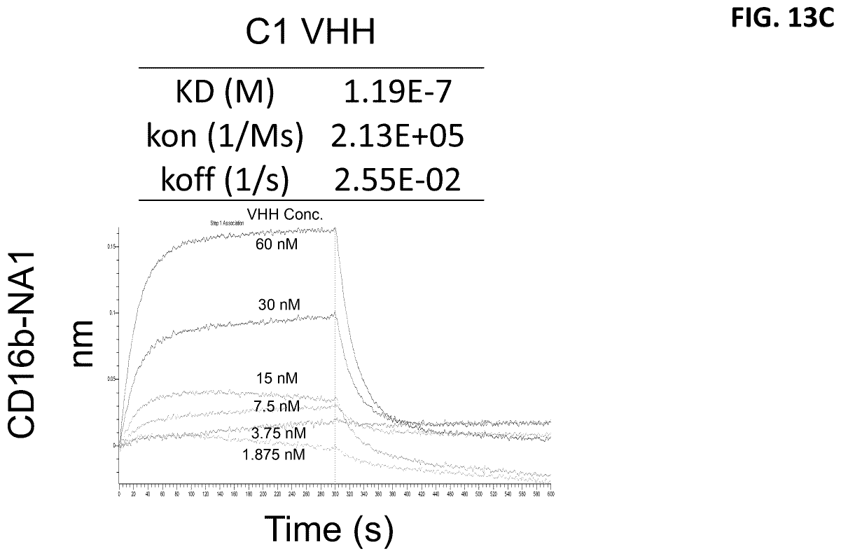

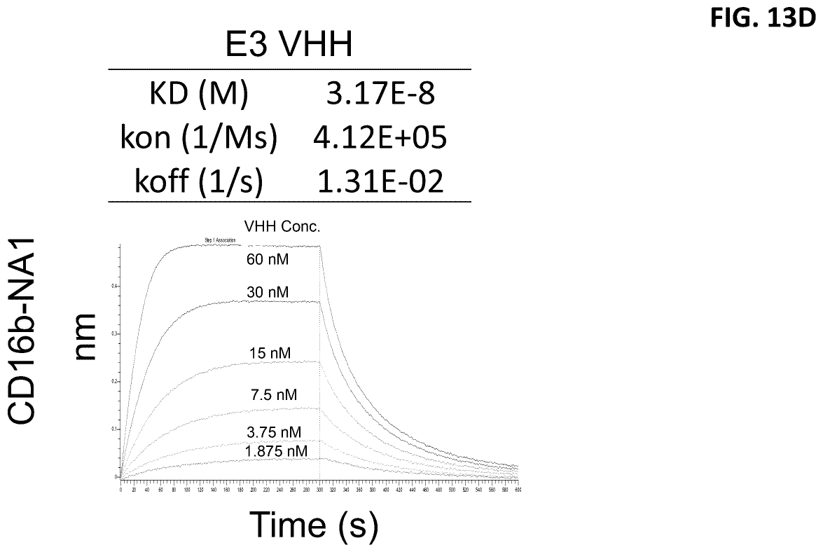

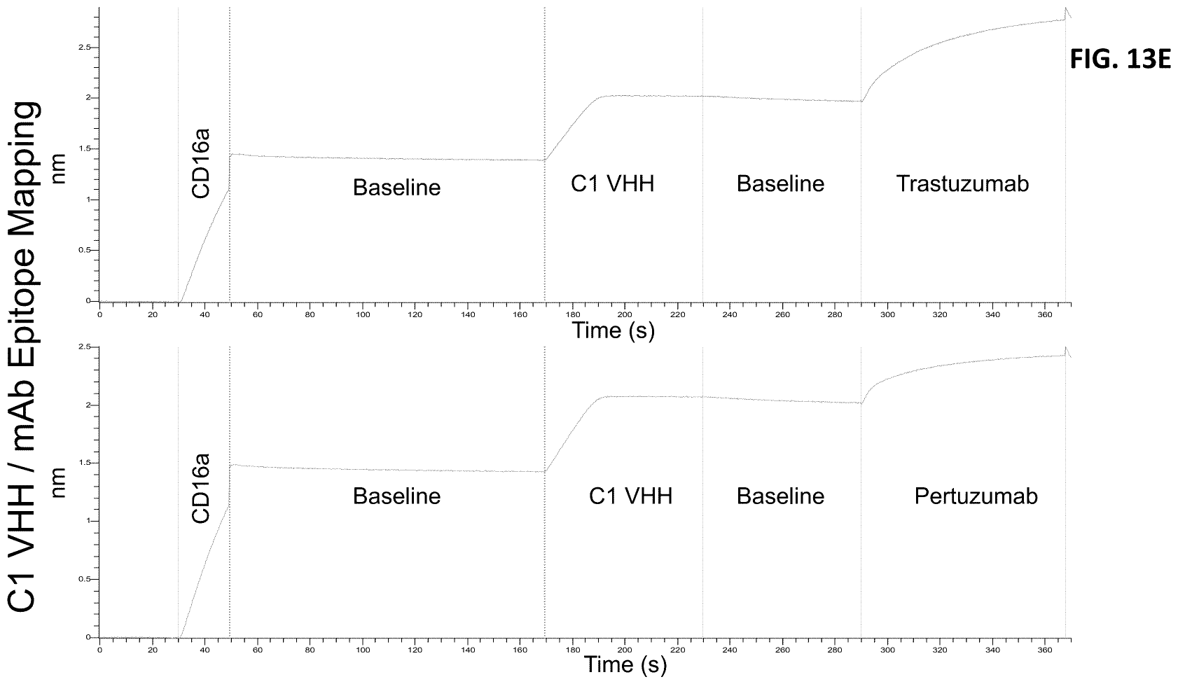

The affinity and specificity of the C1 and E3 VHHs were quantified by BLI (FIGS. 13A-13D). The results of BLI data showed that C1 and E3 VHHs had high affinity (sub-nanomolar level) towards CD16a, whereas their affinities toward CD16b-NA1 were at least 100 folds lower. The epitope mapping data by BLI also showed that the C1 VHH had a different binding site on CD16a than trastuzumab and pertuzumab (FIG. 13E). Overall, the BLI, ELISA and flow cytometry data showed that the selected C1 and E3 VHHs specifically interacted with CD16a without significant interaction with CD16b-NA1 and CD32b.

### Examples: Section 3

### Materials and Methods

### Materials Used for the Production and Characterization of E5C1 BIKE (BIKE: HER2/CD16a)

The list of cells, antibodies and reagents used in this study are listed in Table 17.

TABLE 17
Materials used for the production and characterization of E5C1 BiKE

| Item | Vendor | Catalogue No. |
|---|---|---|
| Trypan Blue Solution, 0.4% | Gibco ™ (NY, USA) | 15250061 |
| DPBS, no calcium, no magnesium | Thermo Scientific ™ (MA, USA) | 14190250 |
| Abgene ™ 96 Well Deepwell Storage Plate | Thermo Scientific ™ (MA, USA) | AB0932 |
| Carbenicillin (Disodium) | GoldBio (MO, USA) | C-103-25 |
| Isopropyl β-d-1-thiogalactopyranoside (IPTG) | Teknova (CA, USA) | I3325 |
| Trypsin-EDTA solution | MiliporeSigma (MA, USA) | T4049-100ML |
| Precision Plus Protein ™ Dual Color Standards | Bio-Rad (PA, USA) | 1610374 |
| ELISA Coating Buffer | Bio-Rad (PA, USA) | BUF030C |
| Sucrose | Sigma-Aldrich (MA, USA) | S7903-1KG |
| Ethylenediaminetetraacetic acid disodium salt | Sigma-Aldrich (MA, USA) | E5134-100G |
| dihydrate | | |
| 1-Step ™ Turbo TMB-ELISA Substrate Solution | Thermo Scientific ™ (MA, USA) | 34022 |
| Escherichia coli (Migula) WK6 | ATCC (VA, USA) | ATCC 47078 |
| Terrific Broth | IBI Scientific (IA, USA) | IB49141 |
| LB Miller Broth | IBI Scientific (IA, USA) | IB49030 |
| Miller's LB Agar | IBI Scientific (IA, USA) | IB49100 |
| Nunc MaxiSorp ™ flat-bottom | Invitrogen ™ (MA, USA) | 44-2404-21 |
| Milk powder, non-fat (skimmed milk), Powder | VWR (PA, USA) | 97063-958 |
| Protein LoBind tubes, PCR clean | Eppendorf (MA, USA) | 0030108302 |
| Octet ® Streptavidin (SA) Biosensor | Sartorius (PA, USA) | 18-5019 |
| Pierce ™ BCA Protein Assay Kit | Thermo Scientific ™ (MA, USA) | 23225 |
| TG1 Electrocompetent Cells | Lucigen (WI, USA) | 60502-2 |
| Goat Anti-Rabbit IgG H&L (FITC) | Abcam (MA, USA) | ab6717 |
| Anti-HA tag antibody (HRP) | Abcam (MA, USA) | ab1190 |
| Fetal Bovine Serum, qualified, United States | Gibco ™ (NY, USA) | 26140079 |
| Penicillin-Streptomycin (10,000 U/mL) | Gibco ™ (NY, USA) | 15140122 |
| Biotinylated Human Fc gamma RIIIA/CD16a | Acrobiosystems (DE, USA) | CDA-H82E8-25ug |
| (F176) Protein, Avitag ™, His Tag (SPR & BLI | | |
| verified) | | |
| Biotinylated Human Fc gamma RIIIB/CD16b | Acrobiosystems (DE, USA) | CDB-H82E4-25ug |
| (NA1) Protein, His,Avitag ™ (SPR & BLI verified) | | |
| Biotinylated Human Fc gamma RIIIB/CD16b | Acrobiosystems (DE, USA) | CDB-H82Ea-25ug |
| (NA2) Protein, His, Avitag ™ (SPR & BLI verified) | | |
| MEM α, no nucleosides | Gibco ™ (NY, USA) | 12561056 |
| Fetal Bovine Serum, qualified, United States | Gibco ™ (NY, USA) | 26140079 |
| Horse Serum, New Zealand origin | Gibco ™ (NY, USA) | 16050122 |
| myo-Inositol | Sigma-Aldrich (MA, USA) | I7508-100G |
| Folic Acid | Sigma-Aldrich (MA, USA) | F8758-5G |
| Penicillin-Streptomycin (10,000 U/mL) | Gibco ™ (NY, USA) | 15140122 |
| Acetic acid (17.48 M) | Sigma-Aldrich (MA, USA) | A6283-100ML |
| Hydrochloric acid solution | Sigma-Aldrich (MA, USA) | H9892-100ML |
| Animal-Free Recombinant Human IL-2 | Peprotech (NJ, USA) | AF-200-02-250UG |
| 2-Mercaptoethanol | Gibco ™ (NY, USA) | 21985023 |
| 0.2 μm Syringe filters with acrylic housing | VWR (PA, USA) | 28145-501 |
| Genetically modified natural killer (NK-92 ®) cell | ATCC (VA, USA) | PTA-8836 |
| Line retroviral transduced to express Human CD16 | | |
| PE Mouse Anti-Human CD16b | BD Bioscience (NJ, USA) | 550868 |
| RPMI-1640 Medium | Sigma-Aldrich (MA, USA) | R8758-6X500ML |
| CD16 Monoclonal Antibody (3G8), Functional | Thermo Scientific ™ (MA, USA) | 16-0166-82 |
| Grade, eBioscience ™ | | |
| pET-28a-c(+) vectors | Novagen (MA, USA) | 69864-3 |
| alamarBlue ™ HS Cell Viability Reagent | Thermo Scientific ™ (MA, USA) | A50100 |
| 96-well plate | | |
| WELQut Protease (5 U/μL) | Thermo Scientific ™ (MA, USA) | EO0861 |
| Biotinylated Human Her2/ErbB2 Protein, | AcroBiosystem (DE, USA) | HE2-H82E2-25ug |
| His, Avitag ™ | | |
| Trastuzumab (Trazimera TM, Pfizer) | Cancer Institute of New Jersey | N/A |
| | Pharmacy Store | |
| Pertuzumab (Perjeta TM, Genentech) | Cancer Institute of New Jersey | N/A |
| | Pharmacy Store | |
| BT-474 breast cancer cells | ATCC (VA, USA) | HTB-20 ™ |
| SK-BR-3 breast cancer cells | ATCC (VA, USA) | HTB-30 ™ |
| SKOV-3 ovarian cancer cells | ATCC (VA, USA) | HTB-77 ™ |
| Hybri-Care Medium | ATCC (VA, USA) | 46-X |
| DMEM/F-12, GlutaMAX ™ supplement | Gibco ™ (NY, USA) | 10565018 |
| Insulin (10 mg/mL) | Sigma-Aldrich (MA, USA) | I0516-5ML |
| McCoy's 5A (Modified) Medium | Gibco ™ (NY, USA) | 16600108 |
| RPMI-1640 Medium | Sigma-Aldrich (MA, USA) | R8758-6X500ML |
| Biotinylated Human Her2/ErbB2 Protein, | AcroBiosystem (DE, USA) | HE2-H82E2-25ug |
| His, Avitag ™ | | |
| SHuffle ® T7 Express Competent E. coli | New England Biolab ™ (MA, USA) | C3029J |

### Expression and Purification of E5C1 BIKE

The E5C1 BiKE construct was synthesized and then cloned into pET28a expression vector by GenScript (Piscataway, NJ). The plasmid was chemically transformed into SHuffle® T7 Express Competent E. coli. First, the highest expressing transformant was selected by western blot. Next, 750 mL Terrific Broth (TB) 100 μg/mL carbenicillin was inoculated by 50 mL of overnight culture. After the OD₆₀₀ₙₘ of 2.5-3 was obtained, 1 mM IPTG was added to start the expression. The culture was then incubated at 37° C. for 7 h. Afterward, the bacteria were spun down using a centrifuge (10,000×g, 10 min, 4° C.). The pellet was stored at −20° C. overnight. The next day, the pellets were resuspended in 3 mL per gram of pellet using Basal Purification Buffer (1 M NaCl, 100 mM KCl, 50 mM Tris, 20 mM Phosphate Buffer, 0.01% Tween 20, 15 mM imidazole, pH 8) and incubated at 4° C. for 30 min while shaking. Afterward, the bacterial suspension was subjected to sonication (5s on, 3s off, 70% amplitude) for 30 min on ice. Next, the cell debris were pelleted via centrifugation (40,000×g, 4° C., 30 min). After that, the supernatant was loaded onto the Ni-NTA column. The non-specific proteins were washed out by 30 mL of wash buffer (500 mM NaCl, 100 mM KCl, 100 mM Tris, 20 mM Phosphate Buffer, 25 mM imidazole, pH 8). To elute E5C1 BiKE, first, the resins were equilibrated with 20 mL of basal elution buffer (500 mM NaCl, 100 mM KCl, 100 mM Tris, 20 mM Phosphate Buffer, 7% sucrose, 0.01% Tween 20, pH 8). Finally, the proteins were eluted using 20 U/mL of WELQut protease. The quality and quantity of the eluted protein were evaluated by SDS-PAGE and BCA kit, respectively. The molecular weight of the purified BiKE and its monomeric status was evaluated at Rutgers Center for Advanced Biotechnology and Medicine core facility using liquid chromatography/mass spectroscopy (LC-MS).

TABLE 18
The amino acid sequence of the E5C1 BiKE (SEQ ID NO: 20) cloned into pET28a
plasmid with theoretical molecular weight of 33.48 kDa. A C-myc and histag
were designed at C-terminal, respectively.

| | HMA | | Protease | |
|---|---|---|---|---|
| Anti-HER2 VHH (E5) | Linker | Anti-CD16 VHH (C1) | Site | Tags |
| QVQLQESGGGLVQAGGSLRLDCA | PSGQAGA | QVQLQESGGGLVQAGGSLRLSC | WELQ | GSEQKLISEE |
| ASGRTLSSYVVGWFRQAPGKERE | AASESLF | AASGRTFRLYRMGWFRQAPGKE | (SEQ ID | DLHHHHHH |
| VVAAIGWSRTSTFYTDSVKGRFTI | VSNHAS | REFVGSIKMIDGSTLYGDPVKGR | NO: 80) | HHHHHH* |
| SRDNTENTVYLQMNSLKPGDTAV | (SEQ ID | FTISRDNTKFMAYLQMNSLKPED | | (SEQ ID NO: |
| YYCAADSSPRRWDRESDFGSWGQ | NO: 78) | TAVYYCASVSRVTGSYDSWGQG | | 81) |
| GTQVTVSS (SEQ ID NO: 77) | | TQVTVSS (SEQ ID NO: 79) | | |

### Evaluation of the VHH Specificity by Enzyme-Linked Immunosorbent Assay (ELISA)

The purified monovalent VHHs, E5 and C1, and E5C1 BiKE were subjected to functional analysis using ELISA and flow cytometry. For ELISA, 50 μL of biotinylated CD16a was added to the streptavidin-coated plate and incubated for 45 min at RT. Following washing, the 100 μl of the 10-fold serial dilution, from 1000 to 0 nM, of purified VHHs/BiKE were added and incubated at RT for 1 h. Next, 100 μl anti-cMyc tag HRP conjugated secondary antibody (1:10,000 dilutions) was added. Finally, after washing, the substrate was added for color development, and the plate was read using OD of 450 nm.

Evaluation of the Ability of VHHs/E5C1 BiKE to Recognize CD16a+ haNK92 Cells by Using Flow Cytometry

For flow cytometry, CD16a⁺ haNK92 cells were purchased from ATCC and cultured in MEM-α supplemented with 12.5% Fetal Bovine Serum (FBS), 12.5% Horse Serum, 0.2 mM myo-inositol, 0.02 mM Folic Acid, 100 U/ml penicillin-streptomycin, 0.1 mM 2-Mercaptoethanol and 100 U/ml IL-2. First, CD16a+NK92 was harvested and washed once using Dulbecco's phosphate-buffered saline (DPBS) supplemented with 2% FBS (2% DPBS-FBS). Next, all the cells were fixed using 3.7% paraformaldehyde for 20 min at RT, followed by washing twice. For each sample, 0.5×10⁶ cells were stained with the 10-fold serial dilution, from 1000 to 0 nM of purified VHHs/BiKE for 60 min at RT. Following washing three times, the secondary antibody, anti-Histag antibody FITC labeled, was added. After washing, the samples were run on Cytoflex using FL1 channel.

### Measurement of VHHs and E5C1 BiKE Binding Affinity and Kinetics Using BLI

To evaluate the binding affinity (K_{D}) and constant rates of association (Kon) and dissociation (Koff) of VHHs and BiKE, an Octet RED96e (Sartorius) Biolayer Interferometer (BLI) located at Biophysics Core Facility at Princeton University was used. An Octet® Streptavidin (SA) Biosensor was soaked for at least 10 min in DPBS supplemented with 0.1% Casein, biotinylated CD16a, CD16b-NA1, CD16b-NA2, and HER2 antigens were loaded onto the streptavidin (SA) biosensor until 1 nm shift was reached. Next, the sensor was dipped into the washing buffer (DPBS+0.05% Tween 20+0.1% Casein) for 2 min to reach the baseline. Then, the sensor was submerged into wells containing 60, 30, 15, 7.5, 3.75, 1.875, and 0 nM of purified anti-CD16a VHHs or BiKE for 5 min (association step). Then, in the dissociation step, sensors were dipped into the washing buffer for 5 min to acquire data (Table 5). The data were then analyzed using Octet Data Analysis HT 11.1 software. The sensorgrams were subtracted from the reference and fitted into 1:1 binding model for data analysis. Finally, the affinity and kinetics were analyzed using “Association and Dissociation”.

### Evaluation of the ADCC by a Cell Toxicity Assay

The ability of BiKE to induce ADCC was evaluated and compared with mAb trastuzumab and/or pertuzumab. In this experiment, target cancer cells (i.e., SKOV-3, BT474, and JIMT-1) were under either adherent or non-adherent conditions. To do ADCC under the adherent condition, 10⁴ cancer cells were seeded in a tissue culture treated 96-well plate. The next day, 100 nM or 10 pM of BiKE or mAb was added to cells and incubated at 37° C. for 30 min. Next, laNK92 or haNK92 cells were added to target cells to make E:T ratios of 4, 2, 1, 0.5, 0.25, and 0 and incubated for four hours at 37° C. Next, the wells were washed twice using DPBS to remove NK92 cells. Finally, 10% alamarBlue™ HS Cell Viability Reagent was added to the plate and incubated for one to two hours at 37° C. Then, the plate was read using Tecan Infinite M Plex plate reader and the cell viability was calculated.

To do ADCC under the non-adherent condition (in suspension), 5×10³ target cancer cells were mixed with 100 nM of mAb or BiKE and then added to a non-treated 96-well plate. Then, different numbers of haNK92 cells were added to each well to obtain the E:T ratios of 16, 8, 4, 2, 1, 0.5, and 0. The plate was incubated at 37° C. for four hours. Next, alamarBlue™ HS Cell Viability Reagent was added and incubated at 37° C. for two to three hours. Finally, the plate was read by a Tecan Infinite M Plex plate reader and the cell viability was measured using the following formula:

[(Fluorescent Intensity of Test Group−Fluorescent Intensity of NK only)/(Fluorescent Intensity of Target Cells only)]×100

Evaluation of the Release of Cytokines and Cytotoxic Proteins from NK92 Cells During ADCC

SKOV-3 cells were seeded in a tissue culture treated 96-well plate at the density of 10,000 cells per well. The next day, 10 nM BiKE or trastuzumab was added to the plate and incubated at 37° C. for 30 min. Next, effector cells (laNK92 or haNK92) were added at E:T ratio of 4. After two hours (Perforin and Granzyme B) and 24 h (TNF-α and IFN-γ), plates were centrifuged down at 2000 g for 10 min to pellet the cells. Then, the supernatant was transferred into a non-treated 96-well plate. The amount of cytokine release was quantified using Quantikine ELISA kit and Perforin Human ELISA Kit following manufacturer's protocol. The data are presented as mean±SD (n=3).

### Quantification of Degranulation Using Surfaced CD107a (LAMP-1)

First, 10⁴ SKOV-3 cells were seeded in a 96-well plate and incubated overnight. The next day, a serial dilution of BiKE and trastuzumab ranging from 0 to 100 nM were added and incubated for 30 min at 37° C. Then, laNK92 (GFP⁺) or haNK92 was added at E:T ratio of 4 and the plate was incubated at 37° C. for two hours. Next, Fc Blocker was added to the plate and incubated at 37° C. for additional one hour. Afterwards, APC-conjugated anti-CD107a antibody was added and incubated for one hour in an incubator. Finally, the plate was centrifuged down, and the supernatant was discarded. The cells were washed twice to remove excess antibodies and data was acquired by the Beckman Coulter CytoFLEX Cytometer. For data analysis, the GFP⁺ (laNK92) was gated to distinguish between effector and target cells. Next, the surfaced CD107a was quantified on GFP⁺ cell population.

### Results and Discussion

### Construction of E5C1 BiKE and Characterization

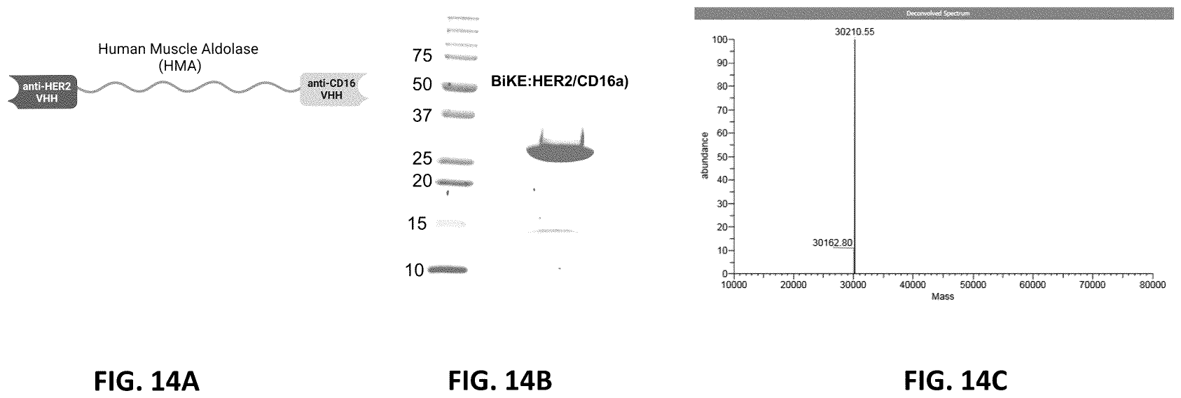

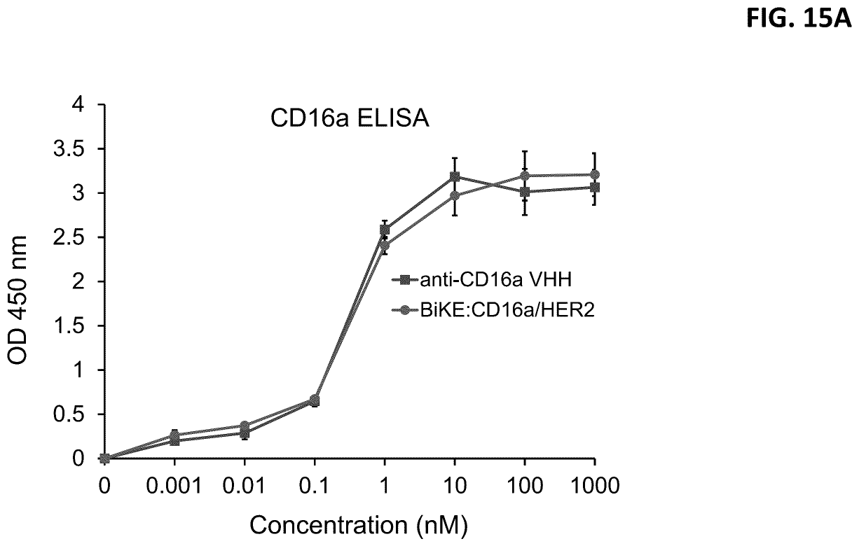

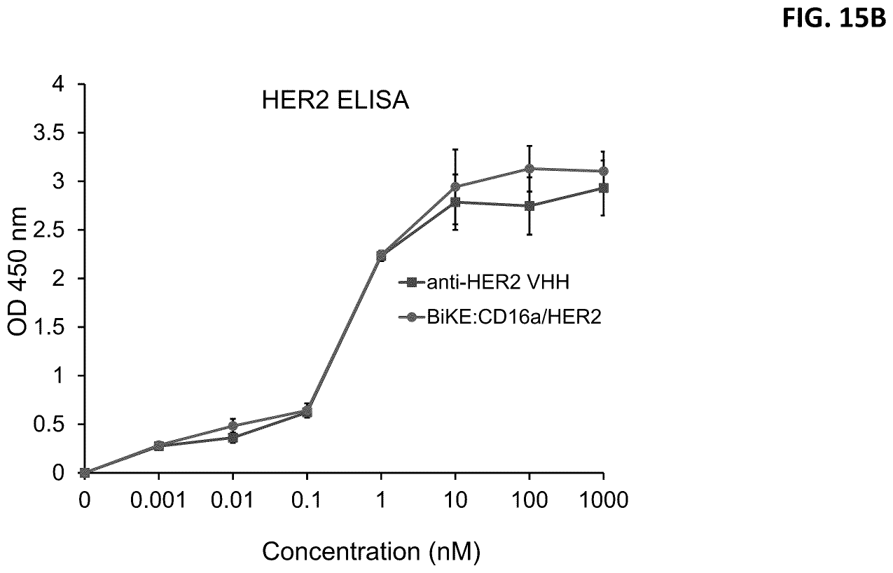

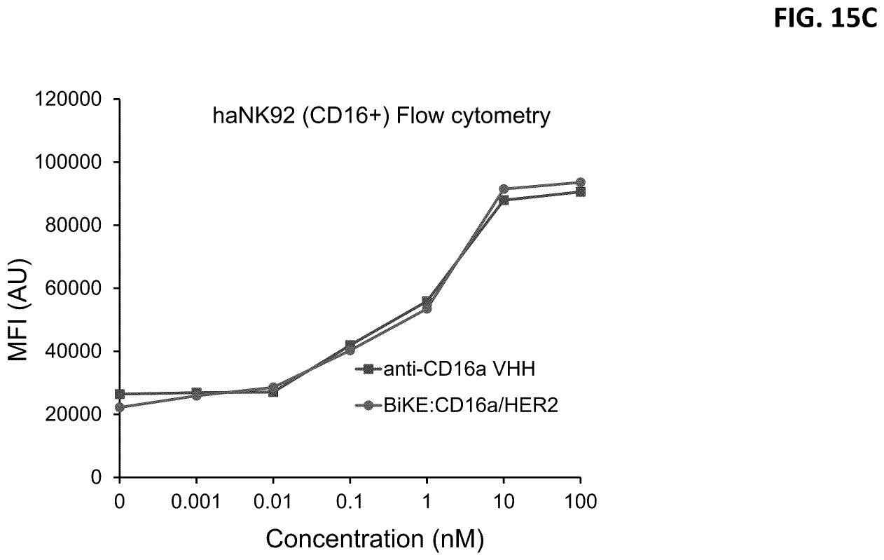

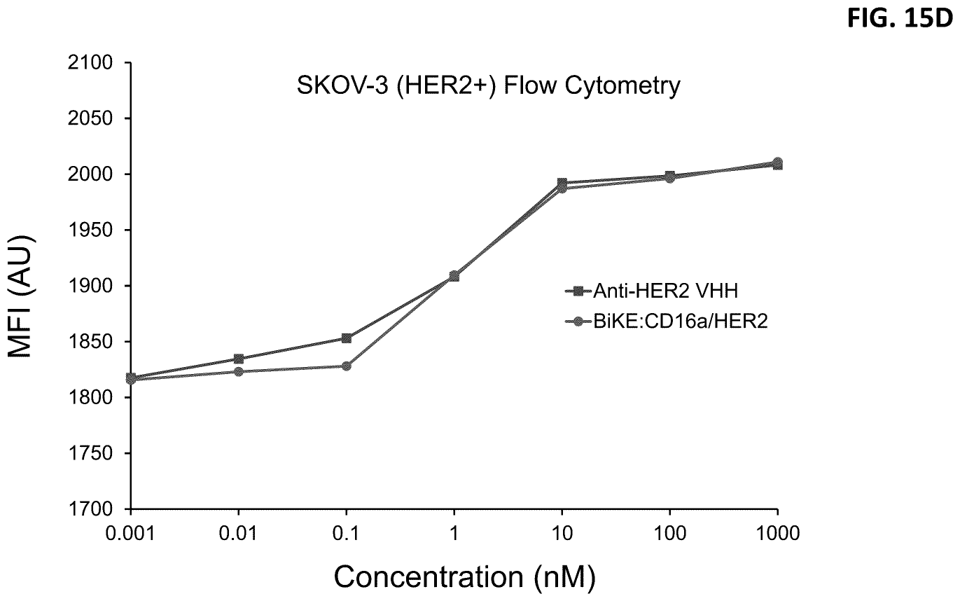

Using recombinant engineering, E5C1 BiKE was constructed by fusing C1 anti-CD16a VHH with E5 anti-HER2 VHH via a HMA semi-flexible linker (FIG. 14A). For simplicity, the construct will be shown as E5C1 BiKE. The SDS-PAGE data showed that the purified E5C1 BiKE had above 95% purity, while the LC-MS graph showed the purified BiKE was free from any dimers or multimers (FIGS. 14B-14C). Then, the binding of the E5C1 BiKE toward CD16a and HER2 antigens was evaluated by ELISA and flow cytometry. C1 anti-CD16a VHH and E5 anti-HER2 VHH were used as controls. Statistical analysis of the data (ELISA and flow cytometry) showed that the affinity of the E5C1 BiKE toward CD16a and HER2 antigens remained intact and fusion of the two VHHs via the HMA linker did not negatively impact their bindings to the CD16a and HER2 antigens (t-test, p>0.05) (FIGS. 15A-15D).

### Measuring the Binding Affinity of E5C1 BiKE by Using BLI

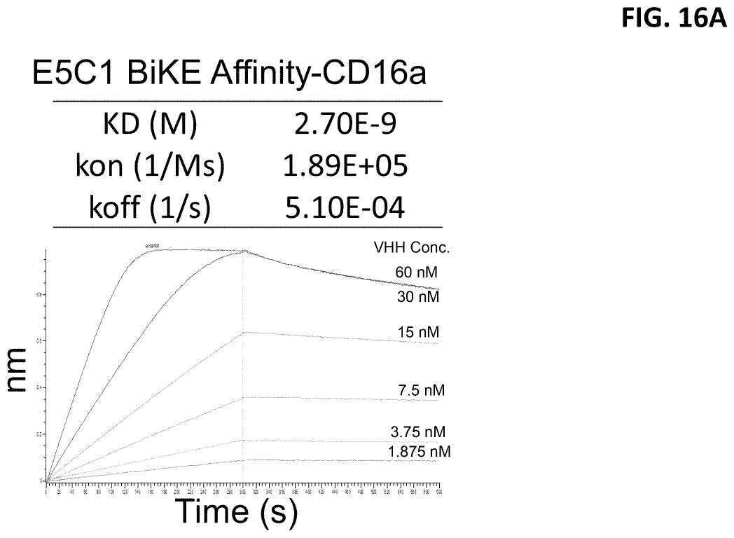

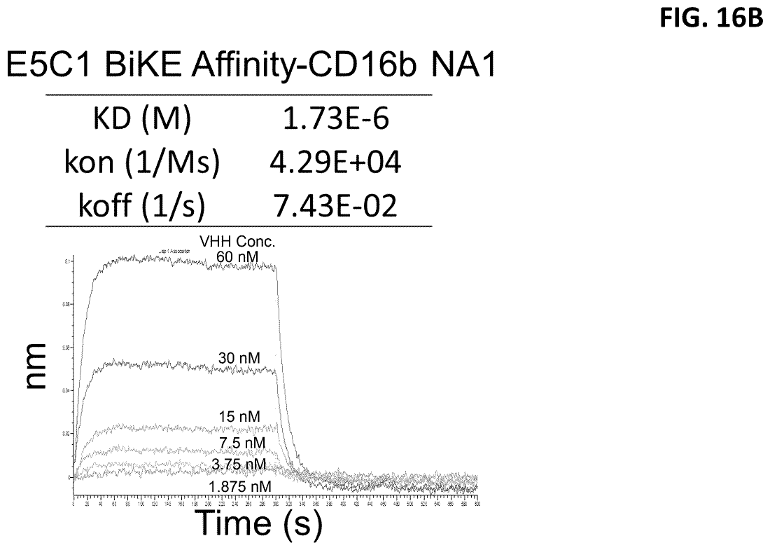

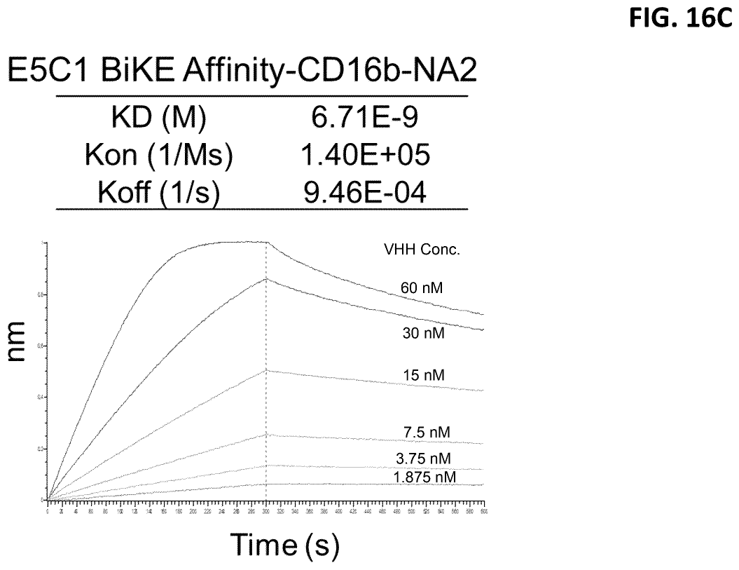

The affinity and specificity of the E5C1 BiKE was measured by BLI. The results of this experiment showed that E5C1 BiKE retained its not only high affinity towards CD16a but also low affinity towards CD16b-NAI antigens (FIGS. 16A-16C). These results confirmed that the affinity and specificity of BiKE construct towards CD16a and CD16b-NA1 remained unchanged.

Evaluation of the Ability of E5C1 BiKE and haNK92 to Kill HER2+ Cancer Cells in Suspension

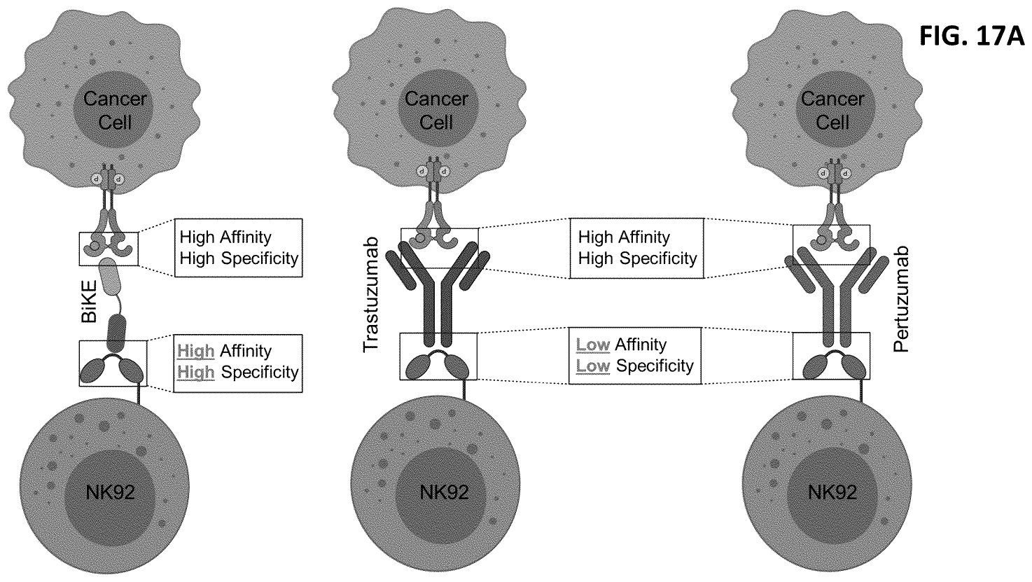

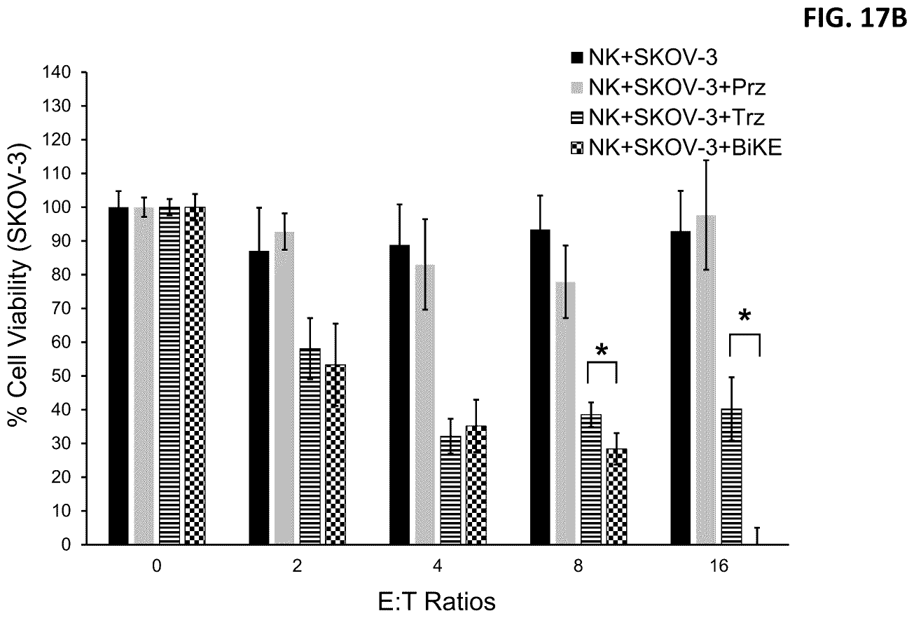

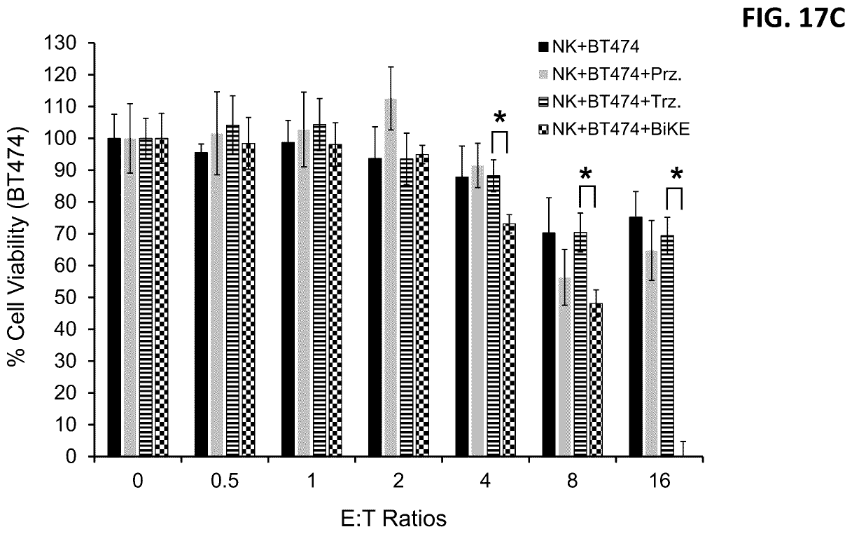

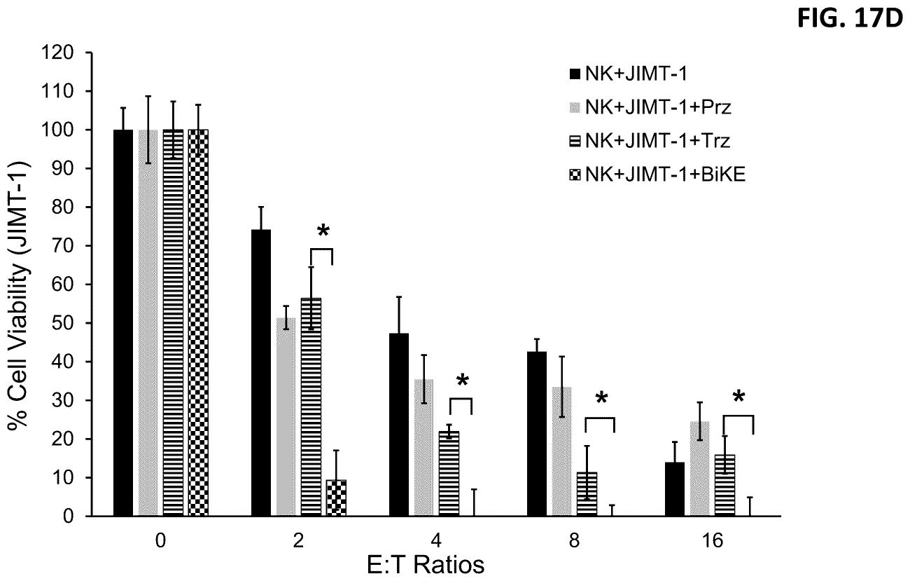

To determine whether E5C1 BiKE can facilitate the recognition and killing of the HER2+ cancer cells in suspension, representing circulating cancer cells, by haNK92 cells, an ADCC assay under non-adherent conditions was performed (FIG. 17A). In this experiment, both cancer cells and haNK92 cells were seeded in non-adherent plates, followed by the addition of either E5C1 BiKE, pertuzumab or trastuzuamb. The results of this study showed E5C1 BiKE assisted in the killing of HER2⁺ cancer cells by haNK92 cells more effectively than trastuzumab and pertuzumab (FIG. 17B-17D). FIG. 17A shows a schematic representation of antibody-directed cell cytotoxicity, in which BiKE, trastuzumab, and pertuzumab not only activate NK cells, but also facilitate recognition of target cancer cells by NK cells.

Measurement of ADCC and Release of Effector Proteins Using E5C1 BIKE, laNK92, and haNK92

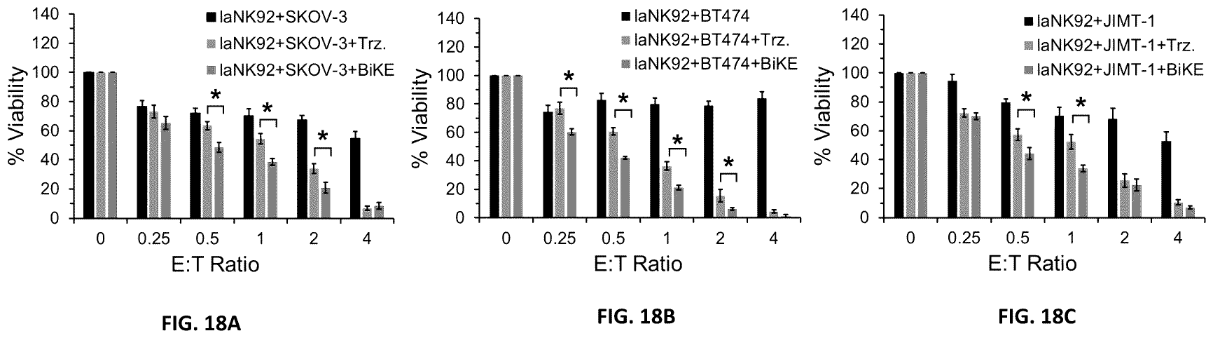

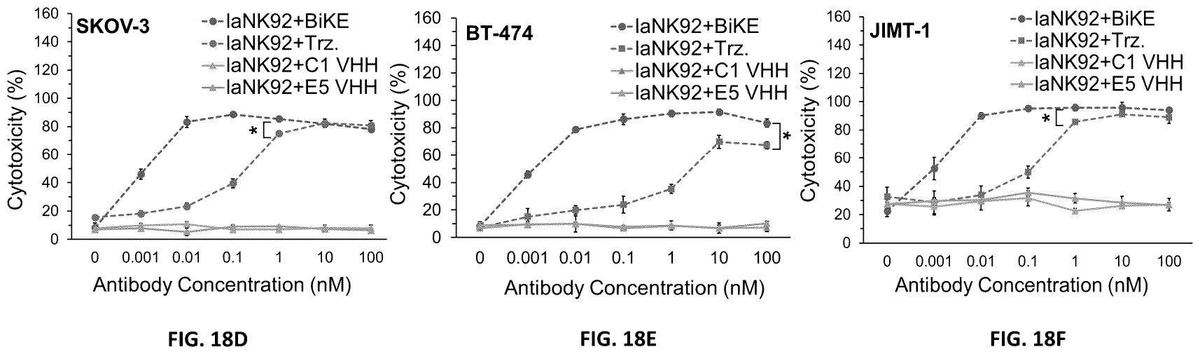

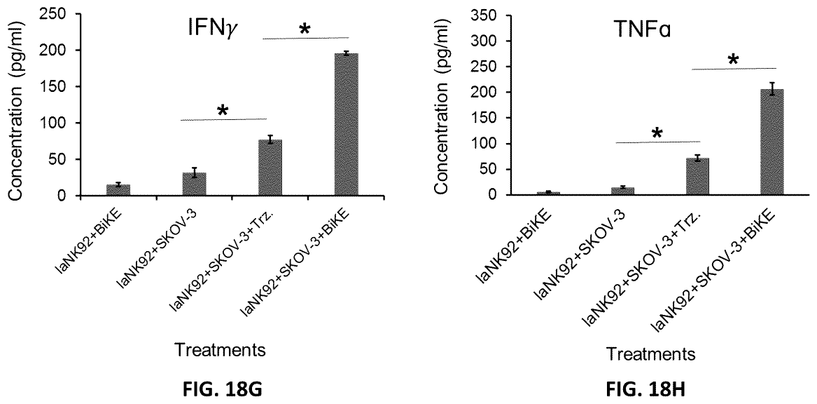

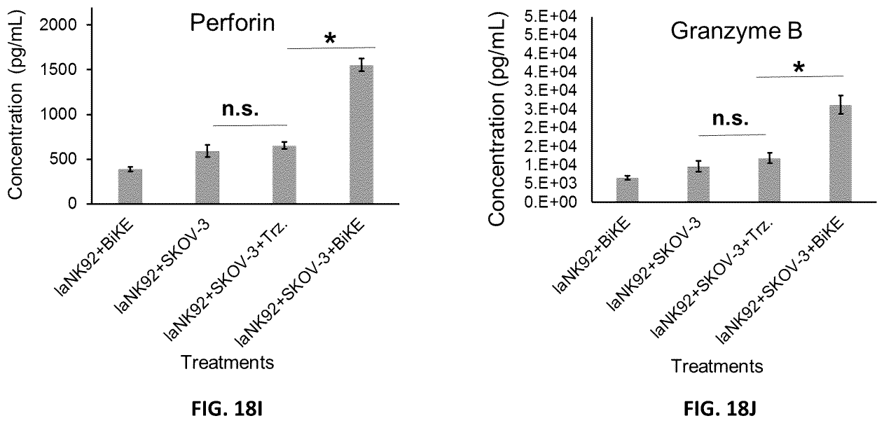

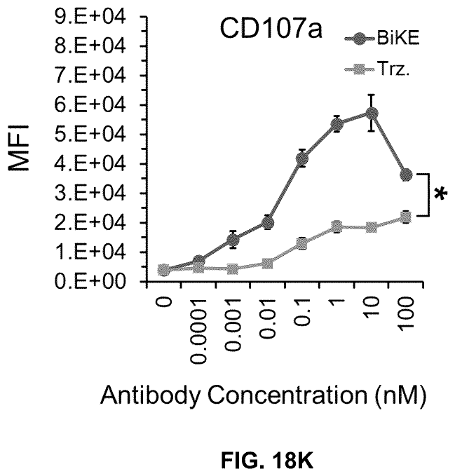

To determine whether E5C1BiKE provides an advantage in terms of ADCC over the currently available best-in-class anti-HER2 mAb (i.e., trastuzumab), a cell toxicity assay was performed. As effector cells, both laNK92 (F176) and haNK92 (V176) cells were used. It has been shown that the CD16a (V176) has a relatively higher affinity toward the Fc region of mAbs. HER2⁺ cancer cell lines SKOV-3, BT474, and JIMT-1 were seeded under adherent conditions and used as target cells. First, cancer cells were treated with laNK92 cells alone, laNK92 plus trastuzumab (equivalent of 100 nM), and laNK92 plus E5C1 BiKE (equivalent of 100 nM) followed by measurement of cell viability. The results of this experiment showed that trastuzumab significantly increased cytotoxicity of laNK92 cells at most E:T ratios; however, in all three tested cell lines, the ADCC of E5C1 BiKE was superior to trastuzumab (FIGS. 18A-18C). Maintaining the ET ratio of 4 at which both trastuzumab and E5C1 BiKE could kill more than 90% of cancer cells, we measured the ADCC using antibodies of different concentrations. The results of this experiment showed that E5C1 BiKE was approximately 100-fold more potent than trastuzumab (FIGS. 18D-18F). To evaluate whether the death of cancer cells was due to stimulation of the laNK92 cells by BiKE, we measured the concentrations of cytotoxic proteins and cytokines, including Perforin, Granzyme B, IFN-γ, and TNF-α during the ADCC experiment. Herein, we used SKOV-3 cells as target cells since our data along with previous literature have shown that SKOV-3 cells have limited expression of NKG2D ligands (i.e., MICA/B) on their surfaces. As a result, the probability of laNK92 cell stimulation through NKG2D ligands on SKOV-3 cells (in the absence of BiKE) is significantly reduced. The results of this experiment showed that BiKE played a significant role in stimulating laNK92 cells to release the cytotoxic proteins and cytokines at rates substantially higher than trastuzumab (FIGS. 18G-18K). The higher rate of laNK92 stimulation with E5C1 BiKE explains the observed higher rate of ADCC in cancer cells, which were treated with laNK92 plus E5C1 BiKE compared to those treated with laNK92 plus trastuzumab. As expected, the data also showed that incubation of laNK92 cells with SKOV-3 (without BiKE) had a limited effect on the release of cytotoxic proteins and cytokines (FIGS. 18G-18K).

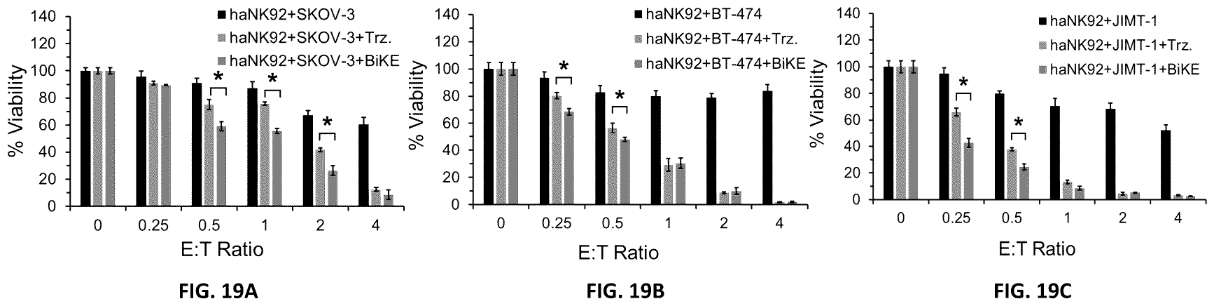

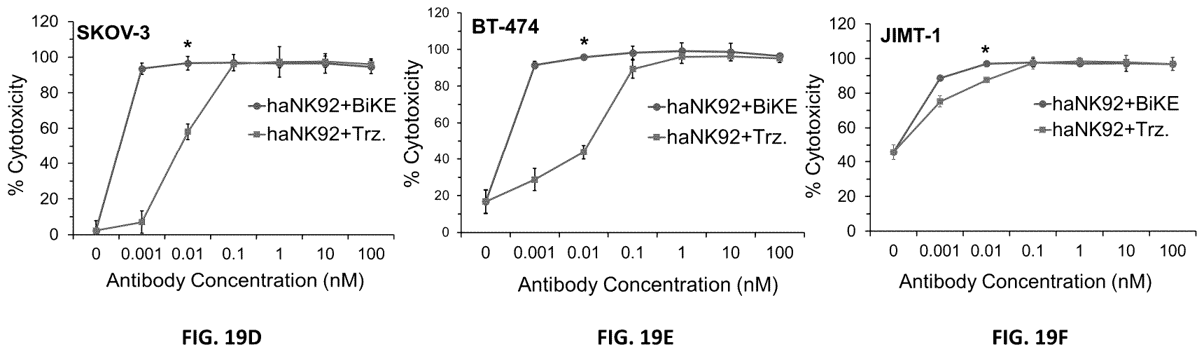

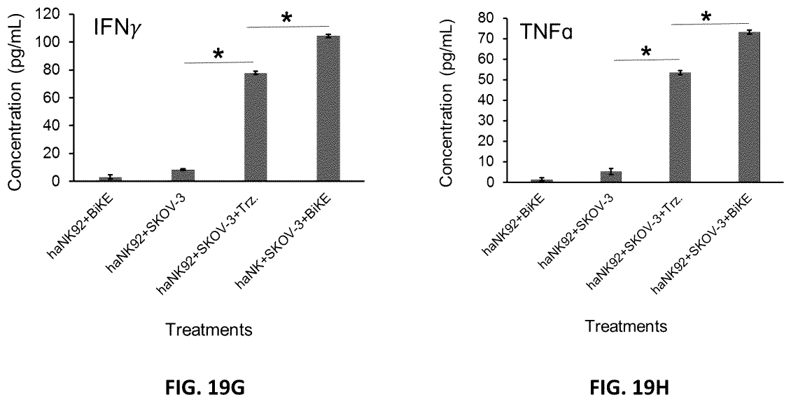

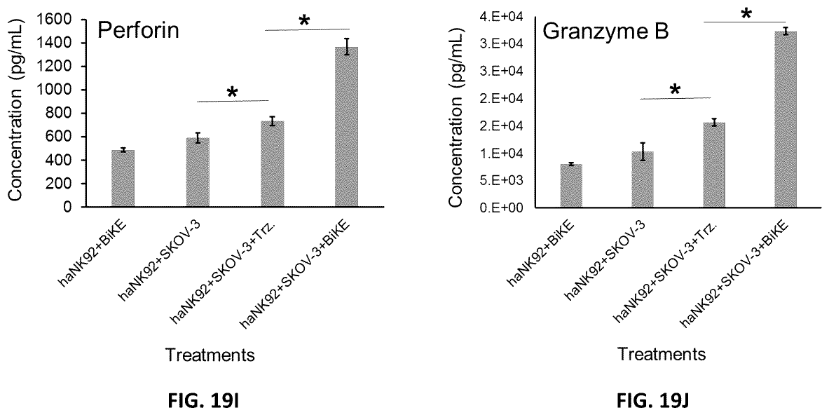

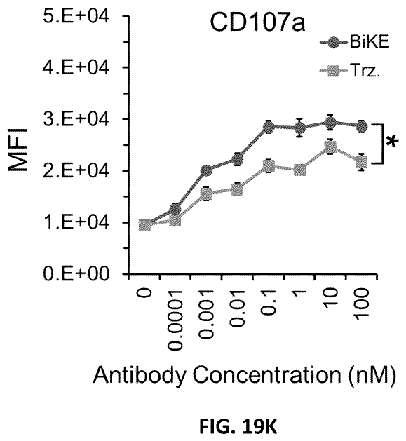

To determine whether E5C1 BiKE provides an advantage in terms of ADCC over trastuzumab in patients with high affinity NK (V176) cells, a cell toxicity assay using haNK92 (V176) was performed. HER2⁺ cancer cells were seeded in adherent condition as mentioned above and treated with haNK92 cells alone, haNK92 plus trastuzumab (equivalent of 100 nM), or haNK92 plus E5C1 BiKE (equivalent of 100 nM), followed by cell viability measurement. Cancer cells were also treated under the same conditions at a fixed E:T ratio using different concentrations of antibodies. The results of these experiments showed that ADCC with E5C1 BiKE was superior to trastuzumab not only at different E:T ratios (FIGS. 19A-19C), but also at different concentrations (FIG. 19D-19F). Measurement of the release of effector proteins also showed that E5C1 BiKE activates haNK92 cells significantly more than trastuzumab (FIGS. 19G-19K).

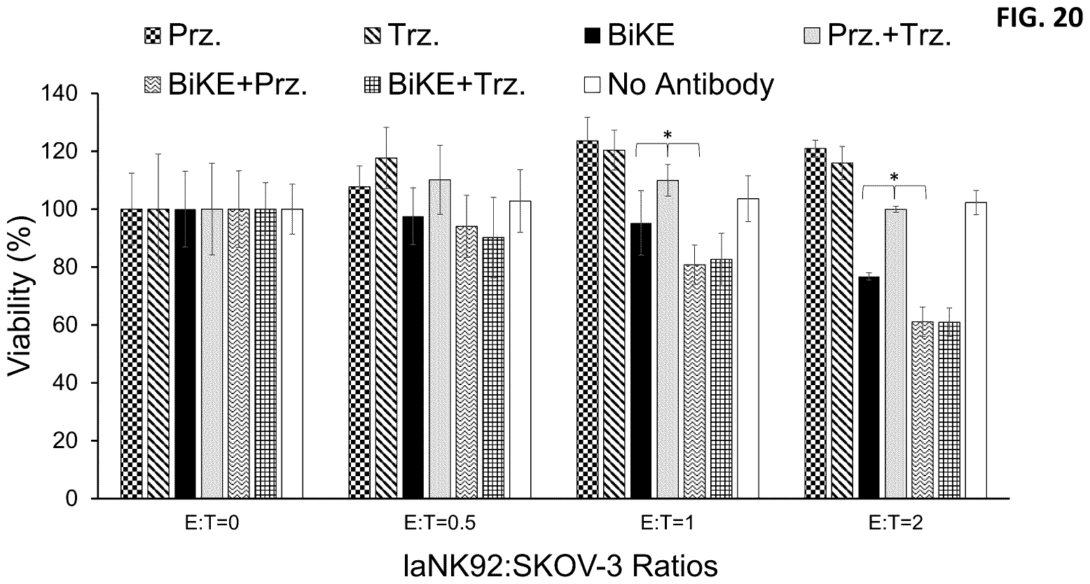

To determine whether E5C1 BiKE provides an advantage in terms of ADCC over trastuzumab plus pertuzumab in patients with low affinity NK cells (F176), a cell toxicity assay using laNK92 was performed. HER2⁺ SKOV-3 cancer cells were seeded in adherent condition as mentioned above and treated with laNK92 cells alone, laNK92 plus trastuzumab (equivalent of 10 pM), laNK92 plus pertuzumab (equivalent of 10 pM), laNK92 plus E5C1 BIKE (equivalent of 10 pM), laNK92 plus trastuzumab plus pertuzumab (equivalent of 10 pM each, total 20 pM), laNK92 plus E5C1 BiKE plus trastuzumab (equivalent of 10 pM each, total 20 pM), or laNK92 plus E5C1 BiKE plus pertuzumab (equivalent of 10 pM each, total 20 pM) followed by cell viability measurement. The results of this experiment showed that E5C1 BiKE alone provided significantly higher level of ADCC in comparison to trastuzumab, pertuzumab, or trastuzumab plus pertuzumab (FIG. 20).

In accordance with one or more embodiments, exemplary single-domain antibodies, methods and uses are set out in the following items:

Item 1. A construct, comprising:
a first single-domain antibody, having an amino acid sequence of one of SEQ ID NOs: 14 and 15, that exhibits specificity and affinity towards the CD16a receptor on the surface of natural killer (NK) cells without cross reactivity with CD16b (e.g., CD16b-NA1) or CD32b; and
a second single-domain antibody having an amino acid sequence that exhibits specificity and affinity toward an antigen associated with a cancer cell, bacteria, parasite, or virus,
wherein the first and second single-domain antibodies are fused with each other with or without a linker.
   Item 2. The construct of item 1, wherein the antigen is associated with a cancer cell, and wherein the antigen is selected from the group consisting of: HER2, HER1, HER3, HER4, EGFR, VEGFR, CD47, FGFR, carcinoembryonic antigen (CEA), Bladder Tumor Antigen (BTA), CA125, PDGFR, IGFR, CA15-3/CA27.29, CA19-9, CA27.29, programmed death ligand 1 (PD-L1), PD-L2, CTL4, CD3, CD19, CD20, CD22, CD25, CD27, CD30, CD33, CD37, CD38, CD40, CD48, CD52, B7-H3, MICA family, RAET1/ULBP family, HLA-E, TIM-3, LAG-3, V-domain Ig suppressor of T cell activation (VISTA), HVEM, ICOS, 4-1BB, OX40, RANKL and GITR, epithelial and mesenchymal markers of circulating tumor cells, Prostatic Acid Phosphatase (PAP), prostate-specific antigen (PSA), soluble mesothelin-related peptides (SMRP), somatostatin receptor (SR), Urokinase plasminogen activator (uPA), plasminogen activator inhibitor (PAI-1), TCR (e.g., MHC class I or class II molecules), A2a Receptor, glioma-associated antigen, carcinoembryonic antigen (CEA), beta-human chorionic gonadotropin, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, prostein, PSMA, prostate-carcinoma tumor antigen-1 (PCTA-1), MART-1, MAGE, tyrosinase, TRP-1, TRP-2 BAGE, GAGE-1, GAGE-2, RAGE, pl5, ELF2M, neutrophil elastase, ephrinB2, IGF-I receptor, E2A-PRL, H4-RET, IGH-IGK, MYL-RAR, TSP-180, pl85erbB2, p180erbB-3, nm-23HI, TAG-72, CA 19-9, CA 72-4, CAM 17.1, NuMa, beta-Catenin, CDK4, Mum-1, p 15, p 16, 43-9F, 5T4, 791Tgp72, beta-HCG, BCA225, BTAA, CA 15-3CA 27.29BCAA, CA 195, CA 242, CA-50, CAM43, CD68P1, CO-029, G250, Ga733EpCAM, HTgp-175, M344, MA-50, MG7-Ag, MOV18, NB/70K, NY-CO-1, RCAS 1, SDCCAG16, TA-90Mac-2 binding protein\cyclophilin C-associated protein, TAAL6, TAG72, TLP, and TPS.
   Item 3. The construct of item 1, wherein the antigen is associated with bacteria, and wherein the antigen is selected from the group consisting of polysaccharides or peptide antigens associated with P. aeruginosa, S. aureus, Clostridium difficile, Acinetobacter baumannii, and Klebsiella pneumonia.
   Item 4. The construct of item 1, wherein the antigen is associated with a virus, and wherein the antigen is selected from the group consisting of Epstein Barr virus antigens EBVA, human papillomavirus (HPV) antigens E6 and E7, coronavirus surface antigens, influenza virus surface antigens, and HIV surface antigens.
   Item 5. The construct of item 1, wherein the antigen is associated with a parasite, and wherein the antigen is selected from the group consisting of antigens associated with malaria, Leishmaniasis, Chagas Disease, Toxoplasmosis, Schistosomiasis, Cysticercosis, and Strongyloidiasis.
   Item 6. The construct of item 1 or item 2, wherein the first single-domain antibody comprises the amino acid sequence of SEQ ID NO: 14 and the second single-domain antibody comprises the amino acid sequence of SEQ ID NO: 6.
   Item 7. The construct of item 1 or item 2, wherein the second single-domain antibody comprises an amino acid sequence of at least one of SEQ ID NOs: 3-11, and wherein the second single-domain antibody exhibits selectivity and affinity towards HER2 and facilitates recognition of HER2-expressing cancer cells.
   Item 8. The construct of item 7, wherein the HER2-expressing cancer cells are ovarian cancer cells, breast cancer cells, gastric cancer, gastroesophageal cancer, cervical cancer cells, bladder cancer cells, gallbladder cancer cells, testicular cancer cells, uterine cancer cells, or any other HER2-expressing cancer cells.
   Item 9. The construct of any one of items 1-8, wherein the amino acid sequence of the first single-domain antibody exhibits high affinity towards the CD16a receptor of the NK cells.
   Item 10. The construct of item 1, wherein the amino acid sequence of the second single-domain antibody exhibits high affinity towards the antigen associated with the cancer cell, bacteria, parasite, or virus.
   Item 11. The construct of any one of items 1-10, wherein the first and second single-domain antibodies are fused with a linker.
   Item 12. The construct of item 11, wherein the linker is a human muscle aldolase (HMA) linker.
   Item 13. The construct of any one of items 1-12, further comprising at least one additional single-domain antibody having an amino acid sequence that exhibits affinity and specificity toward another epitope on the same antigen or on another antigen and fused to at least one of the first or second single-domain antibody with or without a linker.
   Item 14. The construct of item 13, wherein the at least one additional single-domain antibody is the same type of antibody as the first single-domain antibody.
   Item 15. The construct of item 13, wherein the at least one additional single-domain antibody is the same type of antibody as the second single-domain antibody.
   Item 16. A single-domain antibody, comprising:
an amino acid sequence of at least one of SEQ ID NOs: 14 and 15, wherein the single-domain antibody selectively and with high affinity binds to a CD16a activating receptor on the surface of natural killer (NK) cells, without cross reactivity with CD16b-NA1 or CD32b.
   Item 17. A single-domain antibody, comprising:
an amino acid sequence of at least one of SEQ ID NOs: 3-11, wherein the single-domain antibody exhibits selectivity and high affinity towards HER2 and facilitates recognition of HER2-expressing cancer cells.
   Item 18. A method for inhibiting HER2-positive cancers in a subject, the method comprising:
administering to the subject an effective amount of the construct of any one of items 6-8, wherein the construct activates NK cells in the subject to recognize target HER2-positive cancer cells in the subject.
   Item 19. A method of performing an ELISA assay using a single-domain antibody item 16 or item 17 or a construct of any one of items 1-15, the method comprising:
immobilizing a sample comprising one or more antigens on a solid support, wherein the one or more antigens are selected from HER2 and CD16a;
applying the single-domain antibody over a surface of the sample, wherein the single-domain antibody acts as a primary antibody;
applying a secondary antibody over the surface of the sample, wherein the secondary antibody is linked to an enzyme and is configured recognize the single-domain antibody;
adding a substance containing a substrate of the enzyme's substrate to the sample; and
examining the sample to determine whether there is binding between the single-domain antibody and the one or more antigens, wherein if there was binding by the single-domain antibody to the one or more antigens, the subsequent reaction produces a detectable signal in the sample.
   Item 20. A method of performing a flow cytometry assay using a single-domain antibody of item 17, the method comprising:
suspending a sample containing cancer cells and the single-domain antibody in a fluid;
applying, to the sample, a secondary antibody linked to a fluorescent probe that can bind to the single-domain antibody;
injecting the fluid comprising the sample into a flow cytometer instrument;
analyzing the sample with a flow cytometry analyzer; and
determining whether the cancer cells are HER2+ cancer cells.
   Item 21. A cell imaging method using the single-domain antibody of item 17, the method comprising:
fixing a sample comprising suspected cancer cells on a slide;
applying the single-domain antibody to the sample;
applying, to the sample, a secondary antibody linked to a fluorescent probe that can bind to the single-domain antibody;
examining the sample via a confocal or fluorescent microscope to detect a presence or absence of HER2 expression on the surface of the suspected cancer cells.
   Item 22. The method of item 21, wherein the fluorescently-labeled secondary antibody is an anti-histag antibody or an anti-C-myc tag antibody.
   Item 23. An in vivo cell tracking and imaging method for tracking allogenic or autologous NK cells in a subject using a single-domain antibody of item 16, the method comprising:
administering to the subject an imaging substance conjugated to the single-domain antibody;
performing a whole body-imaging method of the subject to produce an image; and identifying the anatomical location of the NK cells in the image.
   Item 24. The method of item 23, wherein the whole body-imaging method is selected from the group consisting of: magnetic resonance imaging (MRI), positron emission tomography (PET), computed tomography (CT), and single photon emission computed tomography (SPECT).
   Item 25. An in vivo cancer phenotyping method for identifying HER2-expressing cancer lesions in a subject, comprising:
administering to the subject an imaging substance conjugated to a single-domain antibody of item 17;
performing a tumor-imaging method of the subject to produce an image; and identifying HER2-expressing cancer lesions in the image.
   Item 26. The method of item 25, wherein the tumor-imaging method is selected from the group consisting of: magnetic resonance imaging (MRI), positron emission tomography (PET), computed tomography (CT), and single photon emission computed tomography (SPECT).
   Item 27. The single-domain antibody of item 17, wherein the single-domain antibody comprises the amino acid sequence of SEQ ID NO: 6.
   Item 28. The single-domain antibody of item 17, wherein the single-domain antibody comprises the amino acid sequence of SEQ ID NO: 11.
   Item 29. The single-domain antibody of item 17, wherein the single-domain antibody comprises the amino acid sequence of SEQ ID NO: 7.
   Item 30. The single-domain antibody of item 17, wherein the HER2-expressing cancer cells are HER2-expressing ovarian cancer cells.
   Item 31. The single-domain antibody of item 30, wherein the HER2-expressing ovarian cancer cells are from a metastatic lesion.
   Item 32. The single-domain antibody of item 17, wherein the HER2-expressing cancer cells are breast cancer cells, cervical cancer cells, bladder cancer cells, gallbladder cancer cells, testicular cancer cells, uterine cancer cells, or any other HER2-expressing cancer cells.
   Item 33. A bispecific single-domain antibody construct, comprising:
a first single-domain antibody having an amino acid sequence of one of SEQ ID NOs: 14 and 15;
a second single-domain antibody having an amino acid sequence of one of SEQ ID NOs: 3-11,
wherein the first and second single-domain antibodies are fused with each other with or without a linker, and wherein the construct exhibits specificity and high affinity towards the CD16a receptor on the surface of natural killer (NK) cells and HER2, without cross reactivity with CD16b (e.g., CD16b-NA1) or CD32b.
   Item 34. The bispecific single-domain antibody construct of item 33, wherein the first single-domain antibody comprises the amino acid sequence of SEQ ID NO: 14 and the second single-domain antibody comprises the amino acid sequence of SEQ ID NO: 6.
   Item 35. The bispecific single-domain antibody construct of item 34, wherein the first and second single-domain antibodies are fused with a linker.
   Item 36. The bispecific single-domain antibody construct of item 35, wherein the linker is a human muscle aldolase (HMA) linker.
   Item 37. A bispecific single-domain antibody construct, comprising:
an amino acid sequence of SEQ ID NO: 20, wherein the construct exhibits specificity and high affinity towards the CD16a receptor on the surface of natural killer (NK) cells and HER2, without cross reactivity with CD16b (e.g., CD16b-NA1) or CD32b.
   Item 38. A method of performing an ELISA assay using a single-domain antibody of any one of items 16, 17, and 27-32, the method comprising:
immobilizing a sample comprising one or more antigens on a solid support, wherein the one or more antigens are selected from HER2 and CD16a;
applying the single-domain antibody over a surface of the sample, wherein the single-domain antibody acts as a primary antibody;
applying a secondary antibody over the surface of the sample, wherein the secondary antibody is linked to an enzyme and is configured recognize the single-domain antibody;
adding a substance containing a substrate of the enzyme's substrate to the sample; and
examining the sample to determine whether there is binding between the single-domain antibody and the one or more antigens, wherein if there was binding by the single-domain antibody to the one or more antigens, the subsequent reaction produces a detectable signal in the sample.
   Item 39. The method of item 38, wherein the secondary antibody is an anti-histag antibody, an anti-C-myc antibody, or an anti-HAtag antibody.
   Item 40. The method of item 38, wherein the enzyme is horseradish peroxidase (HRP).
   Item 41. The method of item 38, wherein the detectable signal is a color change.
   Item 42. A method of performing a flow cytometry assay using a single-domain antibody of any one of items 17 and 27-32, the method comprising:
suspending a sample containing cancer cells and the single-domain antibody in a fluid;
applying, to the sample, a secondary antibody linked to a fluorescent probe that can bind to the single-domain antibody;
injecting the fluid comprising the sample into a flow cytometer instrument;
analyzing the sample with a flow cytometry analyzer; and
determining whether the cancer cells are HER2+ cancer cells.
   Item 43. A method of performing a flow cytometry assay using a single-domain antibody of item 16, the method comprising:
suspending a sample containing NK cells and the single-domain antibody in a fluid;
applying, to the sample, a secondary antibody linked to a fluorescent probe that can bind to the single-domain antibody;
injecting the fluid comprising the sample into a flow cytometer instrument;
analyzing the sample with a flow cytometry analyzer; and
determining whether the NK cells are CD16a+ NK cells.
   Item 44. The method of item 42 or 43, wherein the secondary antibody is an anti-histag antibody, an anti-C-myc tag antibody or an anti-HAtag antibody.
   Item 45. A cell imaging method using the single-domain antibody of any one of items 17 and 27-32, the method comprising:
fixing a sample comprising suspected cancer cells on a slide;
applying the single-domain antibody to the sample;
applying, to the sample, a secondary antibody linked to a fluorescent probe that can bind to the single-domain antibody;
examining the sample via a confocal or fluorescent microscope to detect a presence or absence of HER2 expression on the surface of the suspected cancer cells.
   Item 46. The method of item 45, wherein the secondary antibody is an anti-histag antibody or an anti-C-myc tag antibody.
   Item 47. An ex-vivo cancer phenotyping method by immunohistochemistry, the method using the single-domain antibody of any one of items 17 and 27-32, and the method comprising:
cryosectioning suspected tumor tissue and fixing the suspected tumor tissue on a slide;
staining the suspected tissue with the single-domain antibody;
applying a fluorescently-labeled secondary antibody;
performing photomicrography using a microscope to detect a presence or absence of HER2 expression in the suspected tumor tissue.
   Item 48. The method of item 47, wherein the fluorescently-labeled secondary antibody is an anti-histag antibody or an anti-C-myc tag antibody.
   Item 49. An in vivo cell tracking and imaging method for tracking allogenic or autologous NK cells in a subject using a single-domain antibody of item 16, the method comprising:
administering to the subject an imaging substance conjugated to the single-domain antibody;
performing a whole body-imaging method of the subject to produce an image; and
identifying the anatomical location of the NK cells in the image.
   Item 50. The method of item 49, wherein the whole body-imaging method is magnetic resonance imaging (MRI).
   Item 51. The method of item 49, wherein the whole body-imaging method is positron emission tomography (PET).
   Item 52. The method of item 49, wherein the whole body-imaging method is computed tomography (CT).
   Item 53. The method of item 49, wherein the whole body-imaging method is single photon emission computed tomography (SPECT).
   Item 54. An in vivo cancer phenotyping method for identifying HER2-expressing cancer lesions in a subject, comprising:
administering to the subject an imaging substance conjugated to a single-domain antibody of any one of items 17 and 27-32;
performing a tumor-imaging method of the subject to produce an image; and identifying HER2-expressing cancer lesions in the image.
   Item 55. The method of item 54, wherein the tumor-imaging method is magnetic resonance imaging (MRI).
   Item 56. The method of item 54, wherein the tumor-imaging method is positron emission tomography (PET).
   Item 57. The method of item 54, wherein the tumor-imaging method is computed tomography (CT).
   Item 58. The method of item 54, wherein the tumor-imaging method is single photon emission computed tomography (SPECT).
   Item 59. A construct for treating HER2-positive cancers, the construct comprising:
a bispecific single-domain antibody construct of any one of items 33-37; and
natural killer (NK) cells that express a CD16a receptor to engage the single-domain antibody,
wherein the construct demonstrates significant anticancer activity towards HER2-positive cancers.
   Item 60. A method for inhibiting HER2-positive cancers in a subject, the method comprising: administering to the subject an effective amount of the bispecific single-domain antibody construct of any one of items 33-37, and natural killer (NK) cells that express a CD16a receptor to engage the single-domain antibody.
   Item 61. A method for inhibiting HER2-positive cancers in a subject, the method comprising:
administering to the subject an effective amount of the bispecific single-domain antibody construct of any one of items 33-37, wherein the bispecific single-domain antibody construct activates NK cells in the subject to recognize target HER2-positive cancer cells in the subject.
   Item 62. A construct, comprising:
a first single-domain antibody, having an amino acid sequence of one of SEQ ID NOs: 14 and 15, that exhibits specificity and affinity towards the CD16a receptor on the surface of macrophages without cross reactivity with CD16b (e.g., CD16b-NA1) or CD32b; and
a second single-domain antibody having an amino acid sequence that exhibits specificity and affinity toward an antigen associated with a cancer cell, bacteria, parasite, or virus,
wherein the first and second single-domain antibodies are fused with each other with or without a linker.
   Item 63. The construct of item 62, wherein the antigen is associated with a cancer cell, and wherein the antigen is selected from the group consisting of: HER2, HER1, HER3, HER4, EGFR, VEGFR, CD47, FGFR, carcinoembryonic antigen (CEA), Bladder Tumor Antigen (BTA), CA125, PDGFR, IGFR, CA15-3/CA27.29, CA19-9, CA27.29, programmed death ligand 1 (PD-L1), PD-L2, CTL4, CD3, CD19, CD20, CD22, CD25, CD27, CD30, CD33, CD37, CD38, CD40, CD48, CD52, B7-H3, MICA family, RAET1/ULBP family, HLA-E, TIM-3, LAG-3, V-domain Ig suppressor of T cell activation (VISTA), HVEM, ICOS, 4-1BB, OX40, RANKL and GITR, epithelial and mesenchymal markers of circulating tumor cells, Prostatic Acid Phosphatase (PAP), prostate-specific antigen (PSA), soluble mesothelin-related peptides (SMRP), somatostatin receptor (SR), Urokinase plasminogen activator (uPA), plasminogen activator inhibitor (PAI-1), TCR (e.g., MHC class I or class II molecules), A2a Receptor, glioma-associated antigen, carcinoembryonic antigen (CEA), beta-human chorionic gonadotropin, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, prostein, PSMA, prostate-carcinoma tumor antigen-1 (PCTA-1), MART-1, MAGE, tyrosinase, TRP-1, TRP-2 BAGE, GAGE-1, GAGE-2, RAGE, pl5, ELF2M, neutrophil elastase, ephrinB2, IGF-I receptor, E2A-PRL, H4-RET, IGH-IGK, MYL-RAR, TSP-180, p185erbB2, p180erbB-3, nm-23HI, TAG-72, CA 19-9, CA 72-4, CAM 17.1, NuMa, beta-Catenin, CDK4, Mum-1, p 15, p 16, 43-9F, 5T4, 791Tgp72, beta-HCG, BCA225, BTAA, CA 15-3CA 27.29BCAA, CA 195, CA 242, CA-50, CAM43, CD68P1, CO-029, G250, Ga733EpCAM, HTgp-175, M344, MA-50, MG7-Ag, MOV18, NB/70K, NY-CO-1, RCAS 1, SDCCAG16, TA-90Mac-2 binding protein\cyclophilin C-associated protein, TAAL6, TAG72, TLP, and TPS.
   Item 64. The construct of item 62, wherein the antigen is associated with bacteria, and wherein the antigen is selected from the group consisting of polysaccharides or peptide antigens associated with P. aeruginosa, S. aureus, Clostridium difficile, Acinetobacter baumannii, and Klebsiella pneumonia.
   Item 65. The construct of item 62, wherein the antigen is associated with a virus, and wherein the antigen is selected from the group consisting of Epstein Barr virus antigens EBVA, human papillomavirus (HPV) antigens E6 and E7, coronavirus surface antigens, influenza virus surface antigens, and HIV surface antigens.
   Item 66. The construct of item 62, wherein the antigen is associated with a parasite, and wherein the antigen is selected from the group consisting of antigens associated with malaria, Leishmaniasis, Chagas Disease, Toxoplasmosis, Schistosomiasis, Cysticercosis, and Strongyloidiasis.
   Item 67. The construct of item 62 or item 63, wherein the first single-domain antibody comprises the amino acid sequence of SEQ ID NO: 14 and the second single-domain antibody comprises the amino acid sequence of SEQ ID NO: 6.
   Item 68. The construct of item 62 or item 63, wherein the second single-domain antibody comprises an amino acid sequence of at least one of SEQ ID NOs: 3-11, and wherein the second single-domain antibody exhibits selectivity and affinity towards HER2 and facilitates recognition of HER2-expressing cancer cells.
   Item 69. The construct of item 68, wherein the HER2-expressing cancer cells are ovarian cancer cells, breast cancer cells, gastric cancer, gastroesophageal cancer, cervical cancer cells, bladder cancer cells, gallbladder cancer cells, testicular cancer cells, uterine cancer cells, or any other HER2-expressing cancer cells.
   Item 70. The construct of any one of items 62-69, wherein the amino acid sequence of the first single-domain antibody exhibits high affinity towards the CD16a receptor of the NK cells.
   Item 71. The construct of item 62, wherein the amino acid sequence of the second single-domain antibody exhibits high affinity towards the antigen associated with the cancer cell, bacteria, parasite, or virus.
   Item 72. The construct of any one of items 62-71, wherein the first and second single-domain antibodies are fused with a linker.
   Item 73. The construct of item 72, wherein the linker is a human muscle aldolase (HMA) linker.
   Item 74. The construct of any one of items 62-73, further comprising at least one additional single-domain antibody having an amino acid sequence that exhibits affinity and specificity toward another epitope on the same antigen or on another antigen and fused to at least one of the first or second single-domain antibody with or without a linker.
   Item 75. The construct of item 74, wherein the at least one additional single-domain antibody is the same type of antibody as the first single-domain antibody.
   Item 76. The construct of item 74, wherein the at least one additional single-domain antibody is the same type of antibody as the second single-domain antibody.
   Item 77. A single-domain antibody, comprising:
an amino acid sequence of at least one of SEQ ID NOs: 14 and 15, wherein the single-domain antibody selectively and with high affinity binds to a CD16a activating receptor on the surface of macrophages, without cross reactivity with CD16b-NA1 or CD32b.
   Item 78. A method for inhibiting HER2-positive cancers in a subject, the method comprising:
administering to the subject an effective amount of the construct of any one of items 67-69, wherein the construct activates NK cells in the subject to recognize target HER2-positive cancer cells in the subject.
   Item 79. A bispecific single-domain antibody construct, comprising:
a first single-domain antibody having an amino acid sequence of one of SEQ ID NOs: 14 and 15;
a second single-domain antibody having an amino acid sequence of one of SEQ ID NOs: 3-11,
wherein the first and second single-domain antibodies are fused with each other with or without a linker, and wherein the construct exhibits specificity and high affinity towards the CD16a receptor on the surface of macrophages and HER2, without cross reactivity with CD16b (e.g., CD16b-NA1) or CD32b.
   Item 80. The bispecific single-domain antibody construct of item 79, wherein the first single-domain antibody comprises the amino acid sequence of SEQ ID NO: 14 and the second single-domain antibody comprises the amino acid sequence of SEQ ID NO: 6.
   Item 81. The bispecific single-domain antibody construct of item 80, wherein the first and second single-domain antibodies are fused with a linker.
   Item 82. The bispecific single-domain antibody construct of item 81, wherein the linker is a human muscle aldolase (HMA) linker.
   Item 83. A bispecific single-domain antibody construct, comprising:
an amino acid sequence of SEQ ID NO: 20, wherein the construct exhibits specificity and high affinity towards the CD16a receptor on the surface of macrophages and HER2, without cross reactivity with CD16b (e.g., CD16b-NA1) or CD32b.
   Item 84. A construct for treating HER2-positive cancers, the construct comprising:
a bispecific single-domain antibody construct of any one of items 79-83; and
macrophages that express a CD16a receptor to engage the single-domain antibody,
wherein the construct demonstrates significant anticancer activity towards HER2-positive cancers.
   Item 85. A method for inhibiting HER2-positive cancers in a subject, the method comprising: administering to the subject an effective amount of the bispecific single-domain antibody construct of any one of items 79-83, and macrophages that express a CD16a receptor to engage the single-domain antibody.
   Item 86. A method for inhibiting HER2-positive cancers in a subject, the method comprising:
administering to the subject an effective amount of the bispecific single-domain antibody construct of any one of items 79-83, wherein the bispecific single-domain antibody construct activates macrophages in the subject to recognize target HER2-positive cancer cells in the subject.

### REFERENCES

1. Barb A W. Fc gamma receptor compositional heterogeneity: Considerations for immunotherapy development. J Biol Chem. 2021; 296:100057.
2. Chenoweth A M, Wines B D, Anania J C, Mark Hogarth P. Harnessing the immune system via FcgammaR function in immune therapy: a pathway to next-gen mAbs. Immunol Cell Biol. 2020; 98 (4): 287-304.
3. Koene H R, Kleijer M, Algra J, Roos D, von dem Borne A E, de Haas M. Fc gammaRIIIa-158V/F polymorphism influences the binding of IgG by natural killer cell Fc gammaRIIIa, independently of the Fc gammaRIIIa-48L/R/H phenotype. Blood. 1997; 90(3):1109-14.
4. Treffers L W, van Houdt M, Bruggeman C W, Heineke M H, Zhao X W, van der Heijden J, et al. FcgammaRIIIb Restricts Antibody-Dependent Destruction of Cancer Cells by Human Neutrophils. Front Immunol. 2018; 9:3124.
5. Ocana-Guzman R, Vazquez-Bolanos L, Sada-Ovalle I. Receptors That Inhibit Macrophage Activation: Mechanisms and Signals of Regulation and Tolerance. J Immunol Res. 2018; 2018:8695157.
6. Morris A B, Farley C R, Pinelli D F, Adams L E, Cragg M S, Boss J M, et al. Signaling through the inhibitory Fc receptor FcγRIIB induces CD8+ T cell apoptosis to limit T cell immunity. Immunity. 2020; 52(1):136-50. e6.
7. Arezumand R, Alibakhshi A, Ranjbari J, Ramazani A, Muyldermans S. Nanobodies as novel agents for targeting angiogenesis in solid cancers. Frontiers in immunology. 2017; 8:1746.
8. Ackaert C, Smiejkowska N, Xavier C, Sterckx Y G, Denies S, Stijlemans B, et al. Immunogenicity risk profile of nanobodies. Frontiers in immunology. 2021; 12:578.
9. Allegra A, Innao V, Gerace D, Vaddinelli D, Allegra A G, Musolino C. Nanobodies and cancer: current status and new perspectives. Cancer investigation. 2018; 36(4):221-37.
10. Roberts J T, Barb A W. A single amino acid distorts the Fc gamma receptor IIIb/CD16b structure upon binding immunoglobulin Gl and reduces affinity relative to CD16a. J Biol Chem. 2018; 293(51):19899-908.
11. Nomani A, Li G, Yousefi S, Wu S, Malekshah O M, Nikkhoi S K, et al. Gadolinium-labeled affibody-XTEN recombinant vector for detection of HER2+ lesions of ovarian cancer lung metastasis using quantitative MRI. Journal of Controlled Release. 2021; 337:132-43.
12. Malekshah O M, Sarkar S, Nomani A, Patel N, Javidian P, Goedken M, et al. Bioengineered adipose-derived stem cells for targeted enzyme-prodrug therapy of ovarian cancer intraperitoneal metastasis. Journal of Controlled Release. 2019; 311:273-87.
13. Li J Y, Perry S R, Muniz-Medina V, Wang X, Wetzel L K, Rebelatto M C, et al. A biparatopic HER2-targeting antibody-drug conjugate induces tumor regression in primary models refractory to or ineligible for HER2-targeted therapy. Cancer cell. 2016; 29(1):117-29.

All publications, patents, and patent documents are incorporated by reference herein, as though individually incorporated by reference. This statement of incorporation by reference is intended by applicants, pursuant to 37 C.F.R. § 1.57 (b) (1), to relate to each and every individual publication, patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. No limitations inconsistent with this disclosure are to be understood therefrom.

The invention has been described with reference to various specific and preferred embodiments and techniques. However, it should be understood that many variations and modifications may be made while remaining within the spirit and scope of the invention.

While specific embodiments have been described above with reference to the disclosed embodiments and examples, such embodiments are only illustrative and do not limit the scope of the invention.

1. A construct, comprising:
a first single-domain antibody, having an amino acid sequence of one of SEQ ID NOs: 14 and 15, that exhibits specificity and affinity towards the CD16a receptor on the surface of natural killer (NK) cells without cross reactivity with CD16b (e.g., CD16b-NA1) or CD32b; and
a second single-domain antibody having an amino acid sequence that exhibits specificity and affinity toward an antigen associated with a cancer cell, bacteria, parasite, or virus,
wherein the first and second single-domain antibodies are fused with each other with or without a linker.

2. The construct of claim 1, wherein the antigen is associated with a cancer cell, and wherein the antigen is selected from the group consisting of: HER2, HER1, HER3, HER4, EGFR, VEGFR, CD47, FGFR, carcinoembryonic antigen (CEA), Bladder Tumor Antigen (BTA), CA125, PDGFR, IGFR, CA15-3/CA27.29, CA19-9, CA27.29, programmed death ligand 1 (PD-L1), PD-L2, CTL4, CD3, CD19, CD20, CD22, CD25, CD27, CD30, CD33, CD37, CD38, CD40, CD48, CD52, B7-H3, MICA family, RAET1/ULBP family, HLA-E, TIM-3, LAG-3, V-domain Ig suppressor of T cell activation (VISTA), HVEM, ICOS, 4-1BB, OX40, RANKL and GITR, epithelial and mesenchymal markers of circulating tumor cells, Prostatic Acid Phosphatase (PAP), prostate-specific antigen (PSA), soluble mesothelin-related peptides (SMRP), somatostatin receptor (SR), Urokinase plasminogen activator (uPA), plasminogen activator inhibitor (PAI-1), TCR (e.g., MHC class I or class II molecules), A2a Receptor, glioma-associated antigen, carcinoembryonic antigen (CEA), beta-human chorionic gonadotropin, RAGE-1, MN-CAIX, RU1, RU2 (AS), intestinal carboxyl esterase, mut hsp70-2, M-CSF, prostase, prostate-specific antigen (PSA), PAP, NY-ESO-1, LAGE-1a, prostein, PSMA, prostate-carcinoma tumor antigen-1 (PCTA-1), MART-1, MAGE, tyrosinase, TRP-1, TRP-2 BAGE, GAGE-1, GAGE-2, RAGE, pl5, ELF2M, neutrophil elastase, ephrinB2, IGF-I receptor, E2A-PRL, H4-RET, IGH-IGK, MYL-RAR, TSP-180, p185erbB2, p180erbB-3, nm-23HI, TAG-72, CA 19-9, CA 72-4, CAM 17.1, NuMa, beta-Catenin, CDK4, Mum-1, p 15, p 16, 43-9F, 5T4, 791Tgp72, beta-HCG, BCA225, BTAA, CA 15-3CA 27.29BCAA, CA 195, CA 242, CA-50, CAM43, CD68P1, CO-029, G250, Ga733EpCAM, HTgp-175, M344, MA-50, MG7-Ag, MOV18, NB/70K, NY-CO-1, RCAS 1, SDCCAG16, TA-90Mac-2 binding protein\cyclophilin C-associated protein, TAAL6, TAG72, TLP, and TPS.

3. The construct of claim 1, wherein the antigen is associated with bacteria, and wherein the antigen is selected from the group consisting of polysaccharides or peptide antigens associated with P. aeruginosa, S. aureus, Clostridium difficile, Acinetobacter baumannii, and Klebsiella pneumonia.

4. The construct of claim 1, wherein the antigen is associated with a virus, and wherein the antigen is selected from the group consisting of Epstein Barr virus antigens EBVA, human papillomavirus (HPV) antigens E6 and E7, coronavirus surface antigens, influenza virus surface antigens, and HIV surface antigens.

5. The construct of claim 1, wherein the antigen is associated with a parasite, and wherein the antigen is selected from the group consisting of antigens associated with malaria, Leishmaniasis, Chagas Disease, Toxoplasmosis, Schistosomiasis, Cysticercosis, and Strongyloidiasis.

6. The construct of claim 1, wherein the first single-domain antibody comprises the amino acid sequence of SEQ ID NO: 14 and the second single-domain antibody comprises the amino acid sequence of SEQ ID NO: 6.

7. The construct of claim 1, wherein the second single-domain antibody comprises an amino acid sequence of at least one of SEQ ID NOs: 3-11, and wherein the second single-domain antibody exhibits selectivity and affinity towards HER2 and facilitates recognition of HER2-expressing cancer cells.

8. The construct of claim 7, wherein the HER2-expressing cancer cells are ovarian cancer cells, breast cancer cells, gastric cancer, gastroesophageal cancer, cervical cancer cells, bladder cancer cells, gallbladder cancer cells, testicular cancer cells, uterine cancer cells, or any other HER2-expressing cancer cells.

9. The construct of claim 1, wherein the amino acid sequence of the first single-domain antibody exhibits high affinity towards the CD16a receptor of the NK cells.

10. The construct of claim 1, wherein the amino acid sequence of the second single-domain antibody exhibits high affinity towards the antigen associated with the cancer cell, bacteria, parasite, or virus.

11. The construct of claim 1, wherein the first and second single-domain antibodies are fused with a linker.

12. The construct of claim 11, wherein the linker is a human muscle aldolase (HMA) linker.

13. The construct of claim 1, further comprising at least one additional single-domain antibody having an amino acid sequence that exhibits affinity and specificity toward another epitope on the same antigen or on another antigen and fused to at least one of the first or second single-domain antibody with or without a linker.

14. The construct of claim 13, wherein the at least one additional single-domain antibody is the same type of antibody as the first single-domain antibody.

15. The construct of claim 13, wherein the at least one additional single-domain antibody is the same type of antibody as the second single-domain antibody.

16. A single-domain antibody, comprising:
an amino acid sequence of at least one of SEQ ID NOs: 14 and 15, wherein the single-domain antibody selectively and with high affinity binds to a CD16a activating receptor on the surface of natural killer (NK) cells, without cross reactivity with CD16b-NA1 or CD32b.

17. A single-domain antibody, comprising:
an amino acid sequence of at least one of SEQ ID NOs: 3-11, wherein the single-domain antibody exhibits selectivity and high affinity towards HER2 and facilitates recognition of HER2-expressing cancer cells.

18. A method for inhibiting HER2-positive cancers in a subject, the method comprising:
administering to the subject an effective amount of the construct of claim 6, wherein the construct activates NK cells in the subject to recognize target HER2-positive cancer cells in the subject.

19. A method of performing an ELISA assay using the construct of claim 1, the method comprising:
immobilizing a sample comprising one or more antigens on a solid support, wherein the one or more antigens are selected from HER2 and CD16a;
applying the single-domain antibody over a surface of the sample, wherein the single-domain antibody acts as a primary antibody;
applying a secondary antibody over the surface of the sample, wherein the secondary antibody is linked to an enzyme and is configured recognize the single-domain antibody;
adding a substance containing a substrate of the enzyme's substrate to the sample; and
examining the sample to determine whether there is binding between the single-domain antibody and the one or more antigens, wherein if there was binding by the single-domain antibody to the one or more antigens, the subsequent reaction produces a detectable signal in the sample.

20. A method of performing a flow cytometry assay using a single-domain antibody of claim 17, the method comprising:
suspending a sample containing cancer cells and the single-domain antibody in a fluid;
applying, to the sample, a secondary antibody linked to a fluorescent probe that can bind to the single-domain antibody;
injecting the fluid comprising the sample into a flow cytometer instrument;
analyzing the sample with a flow cytometry analyzer; and
determining whether the cancer cells are HER2+ cancer cells.

21. A cell imaging method using the single-domain antibody of claim 17, the method comprising:
fixing a sample comprising suspected cancer cells on a slide;
applying the single-domain antibody to the sample;
applying, to the sample, a secondary antibody linked to a fluorescent probe that can bind to the single-domain antibody;
examining the sample via a confocal or fluorescent microscope to detect a presence or absence of HER2 expression on the surface of the suspected cancer cells.

22. The method of claim 21, wherein the fluorescently-labeled secondary antibody is an anti-histag antibody or an anti-C-myc tag antibody.

23. An in vivo cell tracking and imaging method for tracking allogenic or autologous NK cells in a subject using a single-domain antibody of claim 16, the method comprising:
administering to the subject an imaging substance conjugated to the single-domain antibody;
performing a whole body-imaging method of the subject to produce an image; and
identifying the anatomical location of the NK cells in the image.

24. The method of claim 23, wherein the whole body-imaging method is selected from the group consisting of: magnetic resonance imaging (MRI), positron emission tomography (PET), computed tomography (CT), and single photon emission computed tomography (SPECT).

25. An in vivo cancer phenotyping method for identifying HER2-expressing cancer lesions in a subject, comprising:
administering to the subject an imaging substance conjugated to a single-domain antibody of claim 17;
performing a tumor-imaging method of the subject to produce an image; and
identifying HER2-expressing cancer lesions in the image.

26. The method of claim 25, wherein the tumor-imaging method is selected from the group consisting of: magnetic resonance imaging (MRI), positron emission tomography (PET), computed tomography (CT), and single photon emission computed tomography (SPECT).


SEQUENCE LISTING (83 sequences)

SEQ ID NO: 1 - protein, 12 aa, synthetic construct
GSEQKLISEEDL

SEQ ID NO: 2 - protein, 12 aa, synthetic construct
HHHHHHHHHHHH

SEQ ID NO: 3 - protein, 144 aa, synthetic construct
QVQLQESGGGLVQAGGSLRISCAASGRTSDLFPVGWFRQAPGKEREIVAAIRWSGGVIGYGDPVKGRFTISTDNAKNTVYLQMNSLKPEDTAVYYCAAGPRESTVTWNYWGQGTHVTVSSGSEQKLISEEDLHHHHHHHHHHHH

SEQ ID NO: 4 - protein, 142 aa, synthetic construct
QVQLQESGGGLVQAGGSLRLSCAASGRTVSRYSMGWFRQAPGKEREFVAAIAWFGGWPYYADSVKGRFTISRDNAKNTVYLQMNSLKPEDTAVYYCAARQWARTWDYWGQGTQVTVSSGSEQKLISEEDLHHHHHHHHHHHH

SEQ ID NO: 5 - protein, 148 aa, synthetic construct
QVQLQESGGGLVQAGGSLTLSCAASGRTFSTYVVGWFRQAPGKERESVAAIGWASNSPYYADSVKGRFTISRDNTKNTVYLQMKSLKPDDTAVYYCAADSYPRRWDRESDFGSWGQGTQVTVSSGSEQKLISEEDLHHHHHHHHHHHH

SEQ ID NO: 6 - protein, 148 aa, synthetic construct
QVQLQESGGGLVQAGGSLRLDCAASGRTLSSYVVGWFRQAPGKEREVVAAIGWSRTSTFYTDSVKGRFTISRDNTENTVYLQMNSLKPGDTAVYYCAADSSPRRWDRESDFGSWGQGTQVTVSSGSEQKLISEEDLHHHHHHHHHHHH

SEQ ID NO: 7 - protein, 148 aa, synthetic construct
QVQLQESGGGSVQAGGSLKLSCAASGRTLSAYVVGWFRQAPGRERESVAAIGWSTHSVYYADSVKGRFTISRDNTENTVYLQMNSLKPDDTAVYYCAADSSPRRWDRESDFGSWGQGTQVTVSSGSEQKLISEEDLHHHHHHHHHHHH

SEQ ID NO: 8 - protein, 148 aa, synthetic construct
QVQLQESGGGLVQAGGSLKLSCAASGRTLSAYVVGWFRQAPGRERESVAAIGWSTHSVYYADSVKGRFTISRDNTENTVYLQMNSLKPDDTAVYYCAADSSPRRWDRESDFGSWGQGTQVTVSSGSEQKLISEEDLHHHHHHHHHHHH

SEQ ID NO: 9 - protein, 148 aa, synthetic construct
QVQLQESGGGLVQAGGSLKLSCAASGRTLSAYVVGWFRQAPGKERESVAAIGWSSRSPYYPDSVKGRFTISEDNTKNTVYLQMNSLKPDDTAVYYCAADSYPRRWDRESDFGSWGQGTQVTVSSGSEQKLISEEDLHHHHHHHHHHHH

SEQ ID NO: 10 - protein, 148 aa, synthetic construct
QVQLQESGGGLVQAGGSLRLSCAASGHTLSTYVVAWFRQAPGKERESVAAIGWSTRSPYYADSVKGRFTISEDNTKNTVYLQMNSLKPDDTAVYYCAADSYPRRWDRESDFGSWGQGTQVTVSSGSEQKLISEEDLHHHHHHHHHHHH

SEQ ID NO: 11 - protein, 148 aa, synthetic construct
QVQLQESGGGLVQPGGSLRLSCAASGHTLSTYVVAWFRQAPGKERESVAAIGWSSRSPYYPDSVKGRFTISEDNTKNTVYLQMNSLKPDDTAVYYCAADSYPRRWDRESDFGSWGQGTQVTVSSGSEQKLISEEDLHHHHHHHHHHHH

SEQ ID NO: 12 - protein, 9 aa, synthetic construct
YPYDVPDYA

SEQ ID NO: 13 - protein, 8 aa, synthetic construct
HHHHHHHH

SEQ ID NO: 14 - protein, 136 aa, synthetic construct
QVQLQESGGGLVQAGGSLRLSCAASGRTFRLYRMGWFRQAPGKEREFVGSIKMIDGSTLYGDPVKGRFTISRDNTKFMAYLQMNSLKPEDTAVYYCASVSRVTGSYDSWGQGTQVTVSSYPYDVPDYAHHHHHHHH

SEQ ID NO: 15 - protein, 136 aa, synthetic construct
QVQLQESGGGLVQAGGSARLSCAASGRTFRLYRMGWFRQAPGKEREFVASIKMIDGSSLFGDPVKGRFTISRDNTKFMVYLQMNSLKPEDTAVYYCASVSRNTGSWDSWGQGTQVTVSSYPYDVPDYAHHHHHHHH

SEQ ID NO: 16 - protein, 7 aa, synthetic construct
PGVQPSP

SEQ ID NO: 17 - protein, 6 aa, synthetic construct
GGGGSG

SEQ ID NO: 18 - protein, 9 aa, synthetic construct
GGGGSGGGS

SEQ ID NO: 19 - protein, 5 aa, synthetic construct
GGGSG

SEQ ID NO: 20 - protein, 291 aa, synthetic construct
QVQLQESGGGLVQAGGSLRLDCAASGRTLSSYVVGWFRQAPGKEREVVAAIGWSRTSTFYTDSVKGRFTISRDNTENTVYLQMNSLKPGDTAVYYCAADSSPRRWDRESDFGSWGQGTQVTVSSPSGQAGAAASESLFVSNHASQVQLQESGGGLVQAGGSLRLSCAASGRTFRLYRMGWFRQAPGKEREFVGSIKMIDGSTLYGDPVKGRFTISRDNTKFMAYLQMNSLKPEDTAVYYCASVSRVTGSYDSWGQGTQVTVSSWELQGSEQKLISEEDLHHHHHHHHHHHH

SEQ ID NO: 21 - protein, 686 aa, synthetic construct
METDTLLLWVLLLWVPGSTGDAAQPARRASLWELQTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTWELQLEEGPHHHHHHHHHHHH

SEQ ID NO: 22 - DNA, 23 bp, synthetic construct
  source 1..23: mol_type = other DNA
gtcctggctgctcttctacaagg

SEQ ID NO: 23 - DNA, 23 bp, synthetic construct
  source 1..23: mol_type = other DNA
ggtacgtgctgttgaactgttcc

SEQ ID NO: 24 - DNA, 41 bp, synthetic construct
  source 1..41: mol_type = other DNA
gatgggctcttctgtgtgcagctgcaggagtctggrggagg

SEQ ID NO: 25 - DNA, 35 bp, synthetic construct
  source 1..35: mol_type = other DNA
ctagtgctcttccgcttggagacggtgacctgggt

SEQ ID NO: 26 - protein, 248 aa, synthetic construct
METDTLLLWVLLLWVPGSTGDAAQPARRASLWELQGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQWELQLEEGPHHHHHHHHHHHH

SEQ ID NO: 27 - protein, 16 aa, synthetic construct
GGGSGGGSGGGSGGGS

SEQ ID NO: 28 - protein, 25 aa, synthetic construct
QVQLQESGGGLVQAGGSLRLSCAAS

SEQ ID NO: 29 - protein, 8 aa, synthetic construct
GRTFRLYR

SEQ ID NO: 30 - protein, 17 aa, synthetic construct
MGWFRQAPGKEREFVGS

SEQ ID NO: 31 - protein, 8 aa, synthetic construct
IKMIDGST

SEQ ID NO: 32 - protein, 38 aa, synthetic construct
LYGDPVKGRFTISRDNTKFMAYLQMNSLKPEDTAVYYC

SEQ ID NO: 33 - protein, 12 aa, synthetic construct
ASVSRVTGSYDS

SEQ ID NO: 34 - protein, 11 aa, synthetic construct
WGQGTQVTVSS

SEQ ID NO: 35 - protein, 25 aa, synthetic construct
QVQLQESGGGLVQAGGSARLSCAAS

SEQ ID NO: 36 - protein, 17 aa, synthetic construct
MGWFRQAPGKEREFVAS

SEQ ID NO: 37 - protein, 8 aa, synthetic construct
IKMIDGSS

SEQ ID NO: 38 - protein, 38 aa, synthetic construct
LFGDPVKGRFTISRDNTKFMVYLQMNSLKPEDTAVYYC

SEQ ID NO: 39 - protein, 12 aa, synthetic construct
ASVSRNTGSWDS

SEQ ID NO: 40 - protein, 25 aa, synthetic construct
QVQLQESGGGLVQPGGSLRLSCAAS

SEQ ID NO: 41 - protein, 8 aa, synthetic construct
GHTLSTYV

SEQ ID NO: 42 - protein, 17 aa, synthetic construct
VAWFRQAPGKERESVAA

SEQ ID NO: 43 - protein, 8 aa, synthetic construct
IGWSSRSP

SEQ ID NO: 44 - protein, 38 aa, synthetic construct
YYPDSVKGRFTISEDNTKNTVYLQMNSLKPDDTAVYYC

SEQ ID NO: 45 - protein, 17 aa, synthetic construct
AADSYPRRWDRESDFGS

SEQ ID NO: 46 - protein, 25 aa, synthetic construct
QVQLQESGGGLVQAGGSLRISCAAS

SEQ ID NO: 47 - protein, 8 aa, synthetic construct
GRTSDLFP

SEQ ID NO: 48 - protein, 17 aa, synthetic construct
VGWFRQAPGKEREIVAA

SEQ ID NO: 49 - protein, 8 aa, synthetic construct
IRWSGGVI

SEQ ID NO: 50 - protein, 38 aa, synthetic construct
GYGDPVKGRFTISTDNAKNTVYLQMNSLKPEDTAVYYC

SEQ ID NO: 51 - protein, 13 aa, synthetic construct
AAGPRESTVTWNY

SEQ ID NO: 52 - protein, 11 aa, synthetic construct
WGQGTHVTVSS

SEQ ID NO: 53 - protein, 8 aa, synthetic construct
GRTVSRYS

SEQ ID NO: 54 - protein, 17 aa, synthetic construct
MGWFRQAPGKEREFVAA

SEQ ID NO: 55 - protein, 8 aa, synthetic construct
IAWFGGWP

SEQ ID NO: 56 - protein, 38 aa, synthetic construct
YYADSVKGRFTISRDNAKNTVYLQMNSLKPEDTAVYYC

SEQ ID NO: 57 - protein, 11 aa, synthetic construct
AARQWARTWDY

SEQ ID NO: 58 - protein, 25 aa, synthetic construct
QVQLQESGGGLVQAGGSLTLSCAAS

SEQ ID NO: 59 - protein, 8 aa, synthetic construct
GRTFSTYV

SEQ ID NO: 60 - protein, 17 aa, synthetic construct
VGWFRQAPGKERESVAA

SEQ ID NO: 61 - protein, 8 aa, synthetic construct
IGWASNSP

SEQ ID NO: 62 - protein, 38 aa, synthetic construct
YYADSVKGRFTISRDNTKNTVYLQMKSLKPDDTAVYYC

SEQ ID NO: 63 - protein, 25 aa, synthetic construct
QVQLQESGGGLVQAGGSLRLDCAAS

SEQ ID NO: 64 - protein, 8 aa, synthetic construct
GRTLSSYV

SEQ ID NO: 65 - protein, 17 aa, synthetic construct
VGWFRQAPGKEREVVAA

SEQ ID NO: 66 - protein, 8 aa, synthetic construct
IGWSRTST

SEQ ID NO: 67 - protein, 38 aa, synthetic construct
FYTDSVKGRFTISRDNTENTVYLQMNSLKPGDTAVYYC

SEQ ID NO: 68 - protein, 17 aa, synthetic construct
AADSSPRRWDRESDFGS

SEQ ID NO: 69 - protein, 25 aa, synthetic construct
QVQLQESGGGSVQAGGSLKLSCAAS

SEQ ID NO: 70 - protein, 8 aa, synthetic construct
GRTLSAYV

SEQ ID NO: 71 - protein, 17 aa, synthetic construct
VGWFRQAPGRERESVAA

SEQ ID NO: 72 - protein, 8 aa, synthetic construct
IGWSTHSV

SEQ ID NO: 73 - protein, 38 aa, synthetic construct
YYADSVKGRFTISRDNTENTVYLQMNSLKPDDTAVYYC

SEQ ID NO: 74 - protein, 25 aa, synthetic construct
QVQLQESGGGLVQAGGSLKLSCAAS

SEQ ID NO: 75 - protein, 8 aa, synthetic construct
IGWSTRSP

SEQ ID NO: 76 - protein, 38 aa, synthetic construct
YYADSVKGRFTISEDNTKNTVYLQMNSLKPDDTAVYYC

SEQ ID NO: 77 - protein, 124 aa, synthetic construct
QVQLQESGGGLVQAGGSLRLDCAASGRTLSSYVVGWFRQAPGKEREVVAAIGWSRTSTFYTDSVKGRFTISRDNTENTVYLQMNSLKPGDTAVYYCAADSSPRRWDRESDFGSWGQGTQVTVSS

SEQ ID NO: 78 - protein, 20 aa, synthetic construct
PSGQAGAAASESLFVSNHAS

SEQ ID NO: 79 - protein, 119 aa, synthetic construct
QVQLQESGGGLVQAGGSLRLSCAASGRTFRLYRMGWFRQAPGKEREFVGSIKMIDGSTLYGDPVKGRFTISRDNTKFMAYLQMNSLKPEDTAVYYCASVSRVTGSYDSWGQGTQVTVSS

SEQ ID NO: 80 - protein, 4 aa, synthetic construct
WELQ

SEQ ID NO: 81 - protein, 24 aa, synthetic construct
GSEQKLISEEDLHHHHHHHHHHHH

SEQ ID NO: 82 - protein, 20 aa, synthetic construct
  VARIANT 1..20: note = This sequence may encompass 1-4 GGGSG repeating units
GGGSGGGGSGGGGSGGGGSG

SEQ ID NO: 83 - protein, 15 aa, synthetic construct
  VARIANT 1..15: note = This sequence may encompass 1-3 GGGSG repeating units
GGGSGGGGSGGGGSG